(19) United States
(12) Patent Application Publication
(10) Pub. No.: US 2025/0375256 A1
(43) Pub. Date: Dec. 11, 2025

(54) METHOD FOR ROBOTICALLY CONTROLLING INTERVENTIONAL DEVICE ASSEMBLY

(71) Applicant: Imperative Care, Inc., Campbell, CA (US)

(72) Inventors: Julie Leigh Berry, Portland, OR (US); Francois Dransart, San Francisco, CA (US); Norio Fujikawa, San Francisco, CA (US); Alan Szu-Han Lai, San Mateo, CA (US); Konrad Marek Guenter Leibrandt, Corona, CA (US); Kelsey Luise Leppek, San Francisco, CA (US); Sean Missal, San Francisco, CA (US); Sarah Plewe, Foster City, CA (US); Tyler Toy, Martinez, CA (US); Chu Han Joy Young, San Francisco, CA (US); Ling Xu, Belmont, CA (US)

(21) Appl. No.: 19/228,468

(22) Filed: Jun. 4, 2025

Related U.S. Application Data
(60) Provisional application No. 63/656,545, filed on Jun. 5, 2024.

(51) Int. Cl. A61B 34/37; A61B 34/00; A61B 34/20; A61B 34/30

(52) U.S. Cl. CPC A61B 34/37; A61B 34/20; A61B 2034/254; A61B 2034/301; A61B 2034/303

(57) ABSTRACT

A robotic interventional device control system includes a first hub assembly coupled to a first interventional device and including a first proximal end and a first distal end; and a second hub assembly coupled to a second interventional device and positioned distal to the first hub assembly. The second hub assembly includes a second proximal end and a second distal end. The system includes a sensor system configured to detect a first position of the first hub assembly and a second position of the second hub assembly; and one or more hardware processors configured to generate a user interface. The user interface includes a drive table window including a first representation of the first hub assembly and a second representation of the second hub assembly.

### INCORPORATION BY REFERENCE TO ANY PRIORITY APPLICATIONS

Any and all applications for which a foreign or domestic priority claim is identified in the application data sheet as filed with the present application are hereby incorporated by reference under 37 C.F.R. § 1.57. The present application claims priority to U.S. Provisional Patent Application No. 63/656,545, filed Jun. 5, 2024, titled METHOD FOR ROBOTICALLY CONTROLLING INTERVENTIONAL DEVICE ASSEMBLY, the entire content of which is incorporated by reference herein for all purposes and forms a part of this specification.

### BACKGROUND

### Field

The present application relates to neurovascular procedures, and more particularly, to catheter assemblies and robotic control systems for neurovascular site access.

### Description of the Related Art

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neurointerventions. Neuro interventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovasculature is challenging to achieve, especially Type III arches. Once supra-aortic access is achieved, adapting the system for neurovascular treatments is time consuming and requires guidewire and access catheter removal and addition of a procedure catheter (and possibly one or more additional catheters) to the stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

There is also provided a method of robotically controlling interventional devices. The method includes providing an interventional device assembly having a plurality of interventional devices arranged in a concentric stack, advancing a subset of the plurality of interventional devices within a vasculature of a patient, and automatically adjusting, via a control system, a velocity of one or more interventional devices of the subset of the plurality of interventional devices to adjust an overall stiffness profile of the concentric stack.

The subset of interventional devices can include a leading interventional device and a trailing interventional device. Advancing the subset of the plurality of interventional devices can include advancing the leading interventional device at a velocity commanded by an actuation of the control of the controller. Automatically adjusting the velocities of one or more interventional devices of the subset of the plurality of interventional devices can include adjusting the velocity of the trailing interventional device.

There is also provided a robotic device control system. The system includes a controller in communication with a first interventional device, a second interventional device, and a third interventional device. The controller includes a joystick, a first actuator configured to be actuated to link the first interventional device, the second interventional device, and the third interventional device with the joystick such that movement of the joystick causes a corresponding movement of each of the first interventional device, the second interventional device, and the third interventional device, a second actuator configured to be actuated to link the second interventional device and the third interventional device with the joystick such that movement of the joystick causes a corresponding movement of each of the second interventional device and the third interventional device, and a third actuator configured to be actuated to selectively link one of the first interventional device, the second interventional device, and the third interventional device with the joystick so that movement of the joystick causes a corresponding movement of the one of the first interventional device, the second interventional device, and the third interventional device.

A first actuation of the third actuator can cause a first one of the first interventional device, the second interventional device, and the third interventional device to be linked with the joystick. A second actuation of the third actuator causes a second one of the first interventional device, the second interventional device, and the third interventional device to be linked with the joystick. A third actuation of the third actuator causes a third one of the first interventional device, the second interventional device, and the third interventional device to be linked with the joystick.

A robotic interventional device control system can include one or more of the features of the description herein.

A method of using the robotic interventional device control system can include one or more features of the description herein.

An interventional device controller can include one or more of the features of the description herein.

A method of using an interventional device controller can include one or more features of the description herein.

A robotic interventional device control system can include one or more of the features of the description herein for use in cardiovascular procedures.

A method of using the robotic interventional device control system can include one or more features of the description herein for use in cardiovascular procedures.

An interventional device controller can include one or more of the features of the foregoing description herein for use in cardiovascular procedures.

Any of the features, components, or details of any of the arrangements or embodiments disclosed in this application, including without limitation any of the controllers, control mechanisms, and user interfaces disclosed below, are interchangeably combinable with any other features, components, or details of any of the arrangements or embodiments disclosed herein to form new arrangements and embodiments.

There is provided in accordance with one aspect of the present disclosure a robotic interventional device control system. The robotic interventional device control system includes a first hub assembly coupled to a first interventional device, the first hub assembly including: a first proximal end; and a first distal end; and a second hub assembly coupled to a second interventional device and positioned distal to the first hub assembly, the second hub assembly including: a second proximal end; and a second distal end; a sensor system configured to detect a first position of the first hub assembly and a second position of the second hub assembly; and one or more hardware processors configured to generate a user interface, the user interface including a drive table window, the drive table window including, a first representation of the first hub assembly, the first representation of the first hub assembly including a first visual indication of the first distal end of the first hub assembly; and a second representation of the second hub assembly, the second representation of the second hub assembly including a second visual indication of the second proximal end of the second hub assembly, wherein the second visual indication of the second proximal end of the second hub assembly is positioned relative to the first visual indication of the first distal end of the first hub assembly based on the detected first and second positions received from the sensor system, thereby the first and second visual indications provide an indication on the user interface how far apart the first distal end of the first hub assembly is from the second proximal end of the second hub assembly; and a display configured to display the user interface.

In some aspects, the first representation of the first hub assembly and the second representation of the second hub assembly can be configured to transition from a first state to a second state upon an occurrence of a control system condition.

The control system condition can include a condition in which the first distal end of the first hub assembly is within a threshold distance of the second proximal end of the second hub assembly.

In some cases, the control system condition can include a condition in which the first distal end of the first hub assembly abuts the second proximal end of the second hub assembly.

In some aspects, the first state can include a first color, texture, or pattern, and wherein the second state includes a second color, texture, or pattern.

The drive table window can be configured to show an alert message upon an occurrence of a control system condition.

In some cases, the control system condition can include a condition in which the first distal end of the first hub assembly is within a threshold distance of the second proximal end of the second hub assembly.

In some aspects, the control system condition can include a condition in which the first distal end of the first hub assembly abuts the second proximal end of the second hub assembly.

The user interface can include an instrument window, wherein the user interface is configured to transition from displaying the instrument window to displaying the drive table window upon an occurrence of the control system condition.

In some cases, the instrument window can include a representation of the first interventional device, said representation of the first interventional device including a visual indication of a distal end of the first interventional device; and a representation of the second interventional device, said representation of the second interventional device including a visual indication of a second distal end of the second interventional device.

In some aspects, the robotic interventional device control system can include a controller having one or more controls configured to cause movement of at least one of the first hub assembly and the second hub assembly.

The robotic interventional device control system can include a status indicator window including; a representation of the first hub assembly; a representation of the second hub assembly; and a linkage indicator configured to provide a visual indication indicating that the first hub assembly and the second hub assembly are both linked to a first control of the controller.

In some cases, the status indicator window can include a drive mode indicator configured to provide a visual indication indicating that the control system is controlling the first hub assembly and the second hub assembly in response to one or more inputs using the controller in accordance with a predefined drive mode.

In some aspects, the predefined drive mode can cause the first hub assembly and the second hub assembly to move simultaneously at different speeds in response to movement of a single control of the controller.

The controller can be configured to control axial movement of the first hub assembly and the second hub assembly along a drive table, wherein the first representation of the first hub assembly is configured to transition from a first configuration to a second configuration when the first hub assembly is moving axially along the drive table.

In some cases, the first representation of the first hub assembly can be configured to transition from a first configuration to a second configuration when movement of the first hub assembly is linked to a first control of the controller.

The robotic interventional device control system can include a drive table, wherein the drive table window includes a representation of the drive table including a visual indication of a first end of the drive table and a visual indication of a second end of the drive table.

In some cases, the sensor system can be configured to detect a position of the drive table.

In some aspects, the representation of the drive table can be configured to move within the drive table window in response to movement of the drive table.

The robotic interventional device control system can include a telescoping member, wherein the drive table window includes a representation of the telescoping member.

There is also provided a robotic interventional device control system. The robotic interventional device control system includes a drive table including a proximal end and a distal end; a hub assembly coupled to an interventional device and positioned on the drive table, wherein the hub assembly is configured to be translated along the drive table, the hub assembly including: a proximal end; and a distal end; and a sensor system configured to detect a position of the drive table and a position of the hub assembly; one or more hardware processors configured to generate a user interface, the user interface including a drive table window, the drive table window including, a drive table representation including a visual indication of the proximal end of the drive table and a visual indication of the distal end of the drive table; a hub assembly representation including a visual indication of the proximal end of the hub assembly and a visual indication of the distal end of the hub assembly, wherein the hub assembly representation is positioned relative to the drive table representation based on the detected position of the drive table and the detected position of the hub assembly received from the sensor system, thereby the visual indications of the proximal end of the hub assembly and the distal end of the hub assembly and the visual indications of the proximal end of the drive table and the distal end of the drive table provide an indication on the user interface how far apart the proximal end of the hub assembly is from the proximal end of the drive table and how far apart the distal end of the hub assembly is from the distal end of the drive table; and a display configured to display the user interface.

In some aspects, the hub assembly representation can be configured to transition from a first state to a second state upon an occurrence of a control system condition.

The first state can include a first color, texture, or pattern, and wherein the second state includes a second color, texture, or pattern.

In some cases, the control system condition can include a condition in which the distal end of the hub assembly has reached the distal end of the drive table.

In some aspects, the control system condition can include a condition in which the distal end of the hub assembly is within a threshold distance of the distal end of the drive table.

The drive table window can be configured to show an alert message upon an occurrence of a control system condition.

In some cases, the user interface can include an instrument window, wherein the user interface is configured to transition from displaying the instrument window to displaying the drive table window upon an occurrence of a control system condition.

The instrument window can include a representation of the interventional device, said representation of the interventional device including a visual indication of a distal end of the interventional device.

There is also provided a robotic interventional device control system. The robotic interventional device control system includes a telescoping member having a proximal end and a distal end; a drive table including a proximal end and a distal end, and configured to be translated along the telescoping member; a sensor system configured to detect a position of the drive table and a position of the telescoping member; one or more hardware processors configured to generate a user interface, the user interface including a drive table window, the drive table window including, a telescoping member representation including a visual indication of the distal end of the telescoping member; and a drive table representation including a visual indication of the proximal end of the drive table and a visual indication of the distal end of the drive table; wherein the drive table representation is positioned relative to the telescoping member representation based on the detected position of the drive table and the detected position of the telescoping member received from the sensor system, thereby providing an indication on the user interface how far apart the distal end of the drive table is from the distal end of the telescoping member; and a display configured to display the user interface.

There is also provided a robotic interventional device control system. The robotic interventional device control system includes a drive table including a proximal end and a distal end; a first hub assembly coupled to a first interventional device and positioned on a drive table, the first interventional device having a first distal end, wherein the first hub assembly is configured to be translated along the drive table, the first hub assembly including: a proximal end; and a distal end; and a second hub assembly coupled to a second interventional device and positioned on the drive table, the second interventional device having a second distal end, wherein the first interventional device is configured to be concentrically nested within the second interventional device wherein the second hub assembly is configured to be translated along the drive table, the second hub assembly including: a proximal end; and a distal end; and a sensor system configured to detect a first position of the first interventional device, a second position of the second interventional device, a third position of the first hub assembly along the drive table, and a fourth position of the second hub assembly along the drive table; one or more hardware processors configured to generate a user interface, the user interface including: an instrument window configured to provide an indication on the user interface regarding how far apart the first distal end of the first interventional device is from the second distal end of the second interventional device; and a drive table window configured to provide an indication on the user interface regarding the position of the first hub assembly and a position of the second hub assembly along the drive table; wherein the user interface is configured to automatically transition from showing the instrument window to showing the drive table window upon an occurrence of a control system condition; and a display configured to display the user interface.

In some aspects, the control system condition can include a condition in which the distal end of the first hub assembly is within a threshold distance of the proximal end of the second hub assembly.

The control system condition can include a condition in which the distal end of the first hub assembly abuts the proximal end of the second hub assembly

In some cases, the user interface can be configured to automatically transition from showing the drive table window to showing the instrument window when the control system condition is resolved.

There is also provided a method of robotically controlling interventional devices. The method includes providing an interventional device assembly including a plurality of interventional devices arranged in a concentric stack; translating a subset of the plurality of interventional devices within a vasculature of a patient; and automatically adjusting, via a control system, a velocity of one or more interventional devices of the subset of the plurality of interventional devices to adjust an overall stiffness profile of the concentric stack.

In some aspects, the subset of the plurality of interventional devices can include a leading interventional device and a trailing interventional device, wherein translating the subset of the plurality of interventional devices includes translating the leading interventional device at a velocity commanded by an actuation of a control of the control system, and wherein automatically adjusting a velocity of the one or more interventional devices of the subset of the plurality of interventional devices includes adjusting the velocity of the trailing interventional device.

Automatically adjusting, via the control system, the velocity of the one or more interventional devices of the subset of the plurality of interventional devices to adjust an overall stiffness profile of the concentric stack can occur during retraction of the one or more interventional devices.

### BRIEF DESCRIPTION OF THE DRAWINGS

FIG. 1 is a schematic perspective view of an interventional setup having an imaging system, a patient support table, and a robotic drive system in accordance with the present disclosure.

FIG. 2 is a longitudinal cross section showing the concentric relationship between a guidewire having two degrees of freedom, an access catheter having 3 degrees of freedom and a guide catheter having one degree of freedom.

FIG. 3A is an exploded schematic view of interventional device hubs separated from a support table by a sterile barrier.

FIGS. 3B-3F show an alternate sterile barrier in the form of a shipping tray having one or more storage channels for carrying interventional devices.

FIGS. 3G-3K show embodiments of an alternate sterile barrier having a convex drive surface.

FIGS. 3L and 3M depict an example of a hub that may be used with the sterile barriers of FIGS. 3G-3K.

FIG. 4 is a schematic elevational cross section through a hub adapter having a drive magnet separated from an interventional device hub and driven magnet by a sterile barrier.

FIGS. 5A and 5B schematically illustrate a three interventional device and a four interventional device assembly.

FIG. 6 illustrates a side elevational schematic view of an interventional device assembly for supra-aortic access and neuro-interventional procedures.

FIGS. 7A-7E depict an example sequence of steps of introducing a catheter assembly configured to achieve supra-aortic access and neurovascular site access.

FIGS. 8A-8B illustrate an example control mechanism for manipulating interventional devices driven by respective hubs.

FIGS. 9-21 illustrate additional embodiments of a user interface for controlling interventional devices.

FIG. 22 illustrates a system diagram of an embodiment of a control system.

FIG. 23A is a simplified block diagram of a medical device operation environment.

FIG. 23B illustrates an embodiment of a process for displaying position and

movement of interventional devices.

FIGS. 24A-24C illustrate an example of a drive mode for a control mechanism.

FIG. 25 illustrates another example of a drive mode for a control mechanism.

FIG. 26 illustrates another example of a drive mode for a control mechanism.

FIG. 27 illustrates another example of a drive mode for a control mechanism.

FIGS. 28A-28B illustrate another example of a control mechanism for manipulating interventional devices driven by respective hubs.

FIGS. 29A-29B illustrate additional examples of a control mechanism for manipulating interventional devices driven by respective hubs.

FIG. 30 illustrates another example of a user interface for controlling interventional devices.

FIGS. 31A-31C illustrate portions of a user interface shown in FIG. 30 corresponding to a drive mode window.

FIGS. 32A-32B illustrate portions of a user interface shown in FIG. 30 corresponding to a drive mode window.

FIG. 33 is perspective view of a hub assembly.

FIG. 34 illustrates an additional example of a control mechanism for manipulating interventional devices driven by respective hubs.

FIG. 35 illustrates another example of a user interface for controlling interventional devices.

FIGS. 36A-36E illustrate portions of a user interface including a toggle window indicator.

FIGS. 37A-37D illustrate portions of a user interface including an instrument window.

FIG. 38 illustrates an example of a telescoping drive table.

FIGS. 39A-39H illustrate portions of a user interface including a drive table window.

### DETAILED DESCRIPTION

In certain embodiments, a system is provided for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations, the system may additionally be configured to robotically gain intra-cranial vascular access and to perform an aspiration thrombectomy or other neuro vascular procedure.

A drive table can be positioned over or alongside the patient, and configured to axially advance, retract, and in some cases rotate and/or laterally deflect two or three or more different (e.g., concentrically or side by side oriented) intravascular devices. The hub is moveable along a path along the surface of the drive table to advance or retract the interventional device as desired. Each hub may also contain mechanisms to rotate or deflect the device as desired, and is connected to fluid delivery tubes (not shown) of the type conventionally attached to a catheter hub. Each hub can be in electrical communication with an electronic control system, either via hard wired connection, RF wireless connection or a combination of both.

Each hub is independently movable across the surface of a sterile field barrier membrane carried by the drive table. Each hub is releasably magnetically coupled to a unique drive carriage on the table side of the sterile field barrier. The drive system independently moves each hub in a proximal or distal direction across the surface of the barrier, to move the corresponding interventional device proximally or distally within the patient's vasculature.

The carriages on the drive table, which magnetically couple with the hubs to provide linear motion actuation, are universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

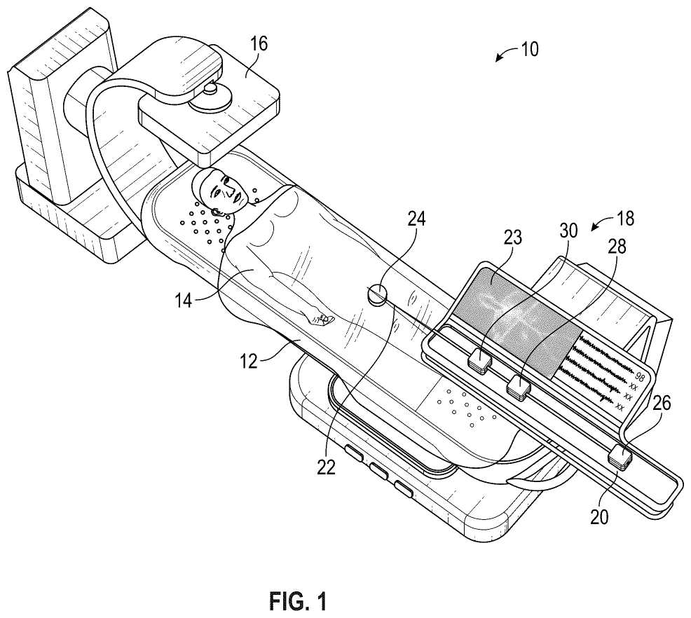

FIG. 1 is a schematic perspective view of an interventional setup 10 having a patient support table 12 for supporting a patient 14. An imaging system 16 may be provided, along with a robotic interventional device drive system 18 in accordance with the present disclosure.

The drive system 18 may include a support table 20 for supporting, for example, a guidewire hub 26, an access catheter hub 28 and a guide catheter hub 30. In the present context, the term ‘access’ catheter can be any catheter having a lumen with at least one distally facing or laterally facing distal opening, that may be utilized to aspirate thrombus, provide access for an additional device to be advanced therethrough or therealong, or to inject saline or contrast media or therapeutic agents.

More or fewer interventional device hubs may be provided depending upon the desired clinical procedure. For example, in certain embodiments, a diagnostic angiogram procedure may be performed using only a guidewire hub 26 and an access catheter hub 28 for driving a guidewire and an access catheter (in the form of a diagnostic angiographic catheter), respectively. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures).

A display 23 such as for viewing fluoroscopic images, catheter data (e.g., fiber Bragg grating fiber optics sensor data or other force or shape sensing data) or other patient data may be carried by the support table 20 and or patient support 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An access catheter hub 28 is also carried by the support table 20 and is movable along the table to advance the access catheter into and out of the patient 14. The access catheter hub may also be configured to rotate the access catheter in response to manipulation of a rotation control, and may also be configured to laterally deflect a deflectable portion of the access catheter, in response to manipulation of a deflection control.

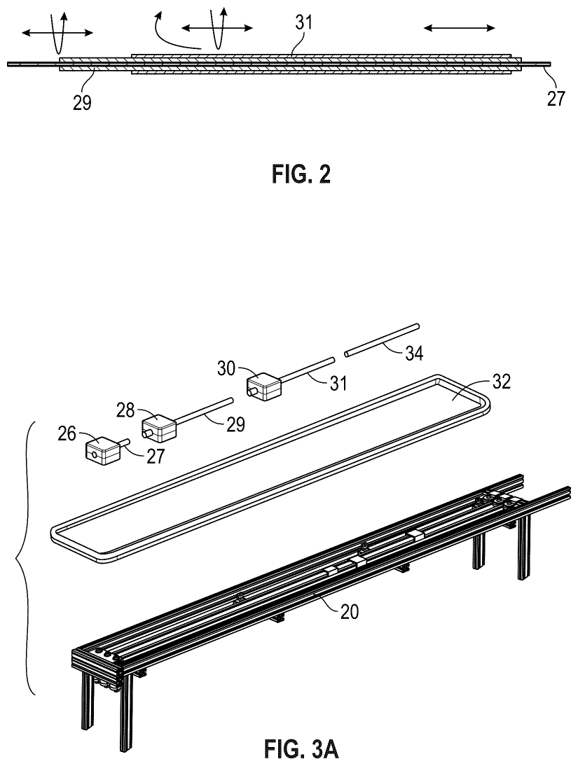

FIG. 2 is a longitudinal cross section schematically showing the motion relationship between a guidewire 27 having two degrees of freedom (axial and rotation), an access catheter 29 having three degrees of freedom (axial, rotational and lateral deflection) and a guide catheter 31, having one degree of freedom (axial).

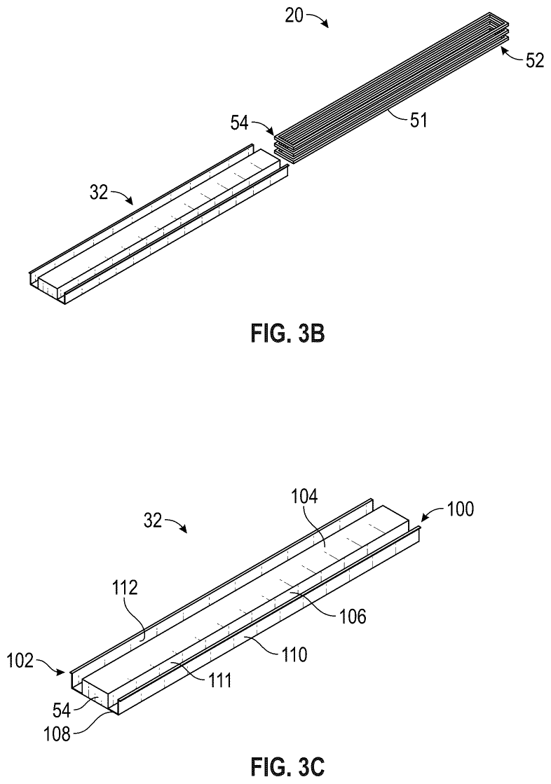

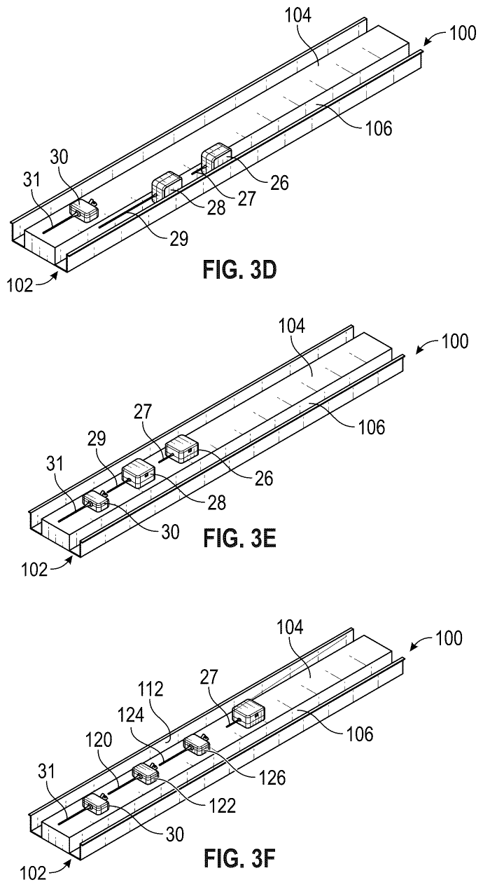

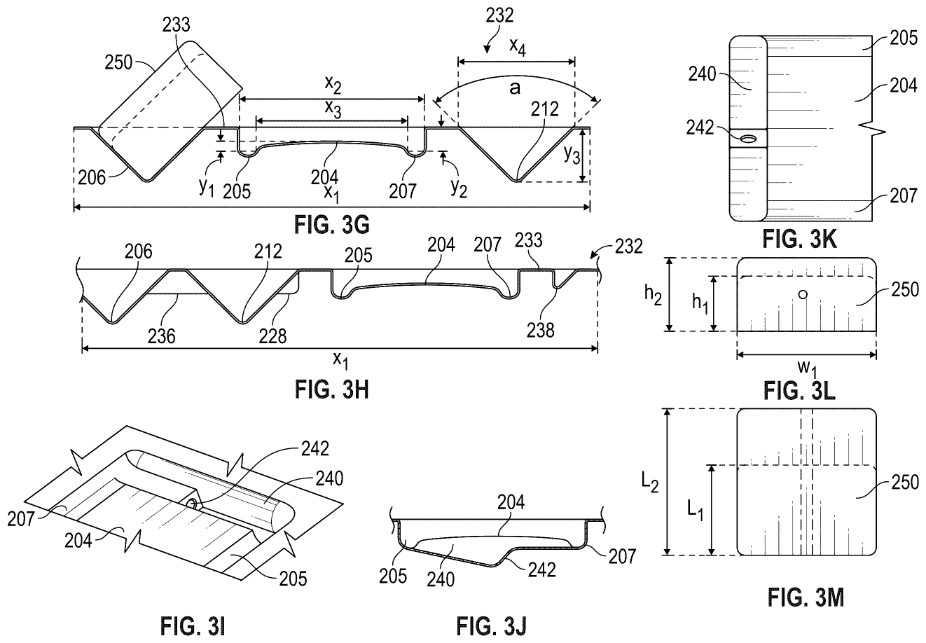

Referring to FIG. 3A, the support table 20 includes a drive mechanism described in greater detail below, to independently drive the guidewire hub 26, access catheter hub 28, and guide catheter hub 30. An anti-buckling feature 34 may be provided in a proximal anti-buckling zone for resisting buckling of the portion of the interventional devices spanning the distance between the support table 20 and the femoral artery access point 24. The anti-buckling feature 34 may include a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 centimeters or 10 centimeters but typically no more than about 120 centimeters or 100 centimeters to support the device between the hub and the access point 24 on the patient. Reinforcement may be accomplished by using metal or polymer tubing or embedding at least one or two or more axially extending elements into the wall of the device shafts, such as elongate wires or ribbons. In some implementations, the extending element may be a hollow and protect from abrasion, buckling, or damage at the inputs and outputs of the hubs. In some embodiments, the hollow extending element may be a hollow and flexible coating attached to a hub. The hollow, extending element (e.g., a hollow and flexible coating) may cover a portion of the device shaft when threaded through the hubs. In some embodiments in which the hollow extending element is a coating, the coating may be attached to a portion of a hub such that threading the catheter device through the hub 26, 28, or 30 threads the catheter device through the coating as well. In some implementations, an anti-buckling device may be installed on or about or surrounding a device shaft to avoid misalignment or insertion angle errors between hubs or between a hub and an insertion point. The anti-buckling device may be a laser cut hypotube, a spring, telescoping tubes, tensioned split tubing, or the like.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts.

Alternatively, thin tubular stiffening structures can be embedded within or carried over the outside of the device wall, such as a tubular polymeric extrusion or length of hypo-tube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, a proximal segment of one or more of the device shafts may be constructed as a tubular hypo tube, which may be machined (e.g., with a laser) so that its mechanical properties vary along its length. This proximal segment may be formed of stainless steel, nitinol, and/or cobalt chrome alloys, optionally in combination with polymer components which may provide for lubricity and hydraulic sealing. In some embodiments, this proximal segment may be formed of a polymer, such as polyether ether ketone (PEEK), polyether ketone ketone (PEKK), polyethylenimine (PEI), or polyimide (PI). Alternatively, the wall thickness or diameter of the interventional device can be increased in the anti-buckling zone.

In certain embodiments, a device shaft having advanced stiffness (e.g., axially and torsionally) may provide improved transmission of motion from the proximal end of the device shaft to the distal end of the device shaft. For example, the device shafts may be more responsive to motion applied at the proximal end. Such embodiments may be advantageous for robotic driving in the absence of haptic feedback to a user.

In some embodiments, a flexible coating can be applied to a device shaft and/or hub to reduce frictional forces between the device shaft and/or hub and a second device shaft when the second device shaft passes therethrough.

The interventional device hubs may be separated from the support table 20 by sterile barrier 32. Sterile barrier 32 may include a thin plastic membrane such as polyethylene terephthalate (PET), polyethylene terephthalate glycol (PETG), polyethylene terephthalate (PETE), high-density polyethylene (HDPE), polyvinyl chloride (PVC), low-density polyethylene (LDPE), polypropylene (PP), polystyrene (PS), or styrene. This allows the support table 20 and associated drive system to reside on a non-sterile (lower) side of sterile barrier 32. The guidewire hub 26, access catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices. The sterile barrier may also act as a sterile work surface for preparation of catheters or other devices during a procedure.

Referring to FIGS. 3B and 3C, there is illustrated a sterile barrier 32 in the form of a pre-shaped tray, for fitting over an elongate support table 20. In use, the elongate support table 20 would be positioned below the sterile barrier 32. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration.

The length of support surface 104 will typically be at least about 100 centimeters and within the range of from about 100 centimeters to about 2.7 meters. Shorter lengths may be utilized in a system configured to advance the drive couplers along an arcuate path. In some embodiments, two or more support surfaces may be used instead of a single support surface 104. The two or more support surfaces may have a combined length between 100 centimeters to about 2.7 meters. The width of the linear drive table is preferably no more than about 30 to about 80 centimeters.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs (for example, by providing lateral support to prevent dislodgment of the hubs when forces are applied to the hubs). First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure as well as to collect fluids and function as wash basins for catheters and related devices.

Referring to FIG. 3D, the guide catheter hub 30 is shown positioned on the upper support surface 104, and magnetically coupled to the corresponding coupler holding the drive magnets, positioned beneath the sterile barrier 32. The access catheter hub 28 and access catheter 29, and guidewire hub 26 and guidewire 27 are illustrated residing within the first channel 106 such as before introduction through the guide catheter 31 or following removal from the guide catheter 31.

The interventional devices may be positioned within the channel 106 and enclosed in a sterile barrier for shipping. At the clinical site, an upper panel of the sterile barrier may be removed, or a tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E, the interventional devices configured in an access assembly for aortic access, following coupling of the access assembly to the corresponding carriages beneath the sterile barrier. The access assembly may be preassembled with the guidewire fully advanced through the access catheter which is in turn fully advanced through the guide catheter. In embodiments in which the access catheter or other catheters are pre-shaped (i.e., pre-curved or not straight), the guidewire and/or outer catheters may be positioned so that relatively stiff sections are not superimposed with curved stiffer sections of the pre-shaped catheter, for example, to avoid creep or straightening of the pre-shaped catheter and/or introduction of a curve into an otherwise straight catheter. This access assembly may be lifted out of the channel 106 and positioned on the support surface 104 for coupling to the respective drive magnets and introduction into the patient. The guide catheter hub 30 is the distal most hub. Access catheter hub 28 is positioned proximally of the guide catheter hub, so that the access catheter 29 can extend distally through the guide catheter. The guidewire hub 26 is positioned most proximally, in order to allow the guidewire 27 to advance through the access catheter 29 and guide catheter 31.

A procedure assembly is illustrated in FIG. 3F following introduction of the procedure assembly through the guide catheter 31 that was used to achieve supra-aortic access. In this implementation, guide catheter 31 remains the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guidewire 27 extends through at least a portion of the second procedure catheter 124 in a rapid exchange version of second procedure catheter 124, or the entire length of second procedure catheter 124 in an over the wire implementation.

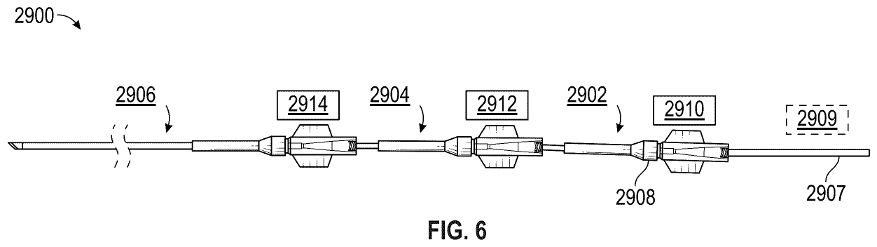

As is discussed in greater detail in connection with FIG. 6, the multi catheter stack may be utilized to achieve both access and the intravascular procedure without the need for catheter exchange. This may be accomplished in either a manual or a robotically driven procedure. In one example, the guide catheter 31 may include a catheter having an inner diameter of at least about 0.08 inches and in one implementation about 0.088 inches. The first procedure catheter 120 may include a catheter having an inner diameter within the range of from about 0.065 inches to about 0.075 inches and in one implementation catheter 120 has an inner diameter of about 0.071 inches. The second procedure catheter 124 may be an access catheter having an OD sized to permit advance through the first procedure catheter 120. The second procedure catheter may be steerable, having a deflection control 2908 configured to laterally deflect a distal end of the catheter. The second procedure (access) catheter may also have an inner lumen sized to allow an appropriately sized guidewire to remain inside the second procedure catheter while performing contrast injections through the second procedure catheter.

In certain embodiments, the catheter 31 may be a ‘large bore’ access catheter or guide catheter having an inner diameter of at least about 0.075 or at least about 0.080 inches in diameter. The catheter 120 may be an aspiration catheter having an inner diameter within the range of from about 0.060 to about 0.075 inches. The catheter 124 may be a steerable catheter with a deflectable distal tip, having an inner diameter within the range of from about 0.025 to about 0.050 inches. The guidewire 27 may have an outer diameter within the range of from about 0.014 to about 0.020 inches. In one example, the catheter 31 may have an inner diameter of about 0.088 inches, the catheter 120 about 0.071 inches, the catheter 124 about 0.035 inches, and the guidewire 27 may have an outer diameter of about 0.018 inches.

In one commercial execution, a preassembled access assembly (guide catheter, access catheter and guidewire) may be carried within a first channel on the sterile barrier tray and a preassembled procedure assembly (one or two procedure catheters and a guidewire) may be carried within the same or a different, second channel on the sterile barrier tray. One or two or more additional catheters or interventional tools may also be provided, depending upon potential needs during the interventional procedure.

FIGS. 3G-3K illustrate embodiments of an alternate sterile barrier having a convex drive surface (e.g., a convex, crowned road like drive surface). FIG. 3G is a cross-sectional view of a sterile barrier 232. The sterile barrier 232 includes a convex upper support surface 204. Fluid channels 205 and 207 are positioned laterally of and below the support surface 204 for self-clearing or draining of fluids from the support surface 204 (for example, during an interventional procedure). The fluid channels 205 and 207 may extend axially at least a portion of the length of the sterile barrier.

FIGS. 3I, 3J, and 3K illustrate a sectional perspective view, a cross-sectional view, and a top sectional view, respectively, of a proximal end of the sterile barrier 232. As shown, in FIGS. 3I-3K, the sterile barrier 232 can include a trough 240 in communication with the fluid channels 205 and 207. The trough 240 can receive fluids from the channels 205 and 207 (for example, during an interventional procedure). The trough 240 may be positioned at least partially below the fluid channels 205 and 207 so that fluid within the channels 205 and 207 flows into the trough 240. In certain embodiments, the fluid channels 205 and 207 may be angled relative to a horizontal plane (for example, may decline from an end of the channel furthest from the trough 240 to the trough 240) so that fluid within the channels 205 and 207 is directed to the trough 240. For example, the channels 205 and 207 may increase in depth from an end of the channels furthest from the trough 240 to the trough 240. Alternatively, the sterile barrier 232 and/or support table may be positioned at an angle relative to a horizontal plane, during part of or an entirety of an interventional procedure, such that the end of the channels 205 and 207 furthest from the trough 240 is positioned higher than the trough 240. For example, the sterile barrier 232 and/or support table may be constructed or arranged in an angled arrangement so that an end of the sterile barrier 232 and/or support table opposite the trough 240 is positioned higher than the trough 240. Alternatively or additionally, a drive mechanism may temporarily tilt the sterile barrier 232 and/or support table so that an end of the sterile barrier 232 and/or support table opposite the trough 240 is positioned higher than the trough 240 (for example, by lifting an end of the sterile barrier and/or support table opposite the trough 240 or lowering an end of the sterile barrier 232 and/or support table at which the trough 240 is positioned) so that fluids within the channels 205 and 207 flow into the trough 240.

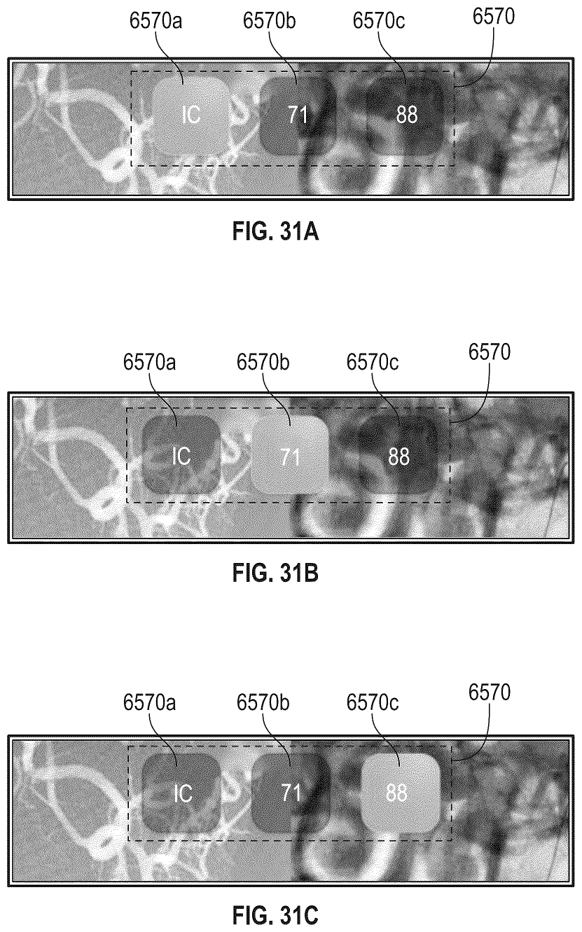

The trough 240 can include a drain hole 242. The trough 240 can be shaped, dimensioned, and/or otherwise configured so that fluid within the trough 240 empties to the drain hole 242. The drain hole 242 can include tubing, a barb fitting, and/or an on-off valve for removal of fluids from the trough 240. As shown in FIGS. 31-3K, the trough 240 can be positioned at the proximal end of the sterile barrier 232. In alternate embodiments, the trough 240 may be positioned at a distal end of the sterile barrier 232. In some embodiments, the sterile barrier 232 can include a first trough 240 at the proximal end and a second trough 240 at the distal end. In some embodiments, the trough 240 can also be used as a wash basin.

A first channel 206 may extend axially at least a portion of the length of the sterile barrier 232. The channel 206 can have a sufficient length to hold the interventional devices, and sufficient width and depth to hold the corresponding hubs (for example, by providing support to prevent dislodgement of the hubs when forces are applied to the hubs). Optionally, a second channel 212 may be provided. The second channel 212 may be located on the same side or the opposite side of the upper support surface 204 from the first channel 206. FIG. 3G illustrates the channel 212 located on the opposite side of the support surface 204 from the channel 206. FIG. 3H is a cross-sectional view illustrating an alternate embodiment of the sterile barrier 232 in which the channel 212 is on the same side of the support surface 204 as the channel 206.

As shown in FIGS. 3G and 3H, the channels 206 and 212 can have generally triangular, wedge-shaped, or otherwise angled cross-sections, so as to hold the hubs at an angle relative to a horizontal plane. Holding the hubs at an angle relative to the horizontal plane can allow for smaller width of the sterile barrier 232.

Two or three or more additional recesses such as additional channels or wells may be provided, to hold additional medical devices or supplies that may be useful during the interventional procedure as well as to collect fluids and function as wash basins for catheters and related devices. In some embodiments, any of the channels or wells described herein may not be part of the sterile barrier, but may instead be part of the drive table positioned below the sterile barrier.

In some embodiments, the sterile barrier 232 can include one or more structural ribs 236. The sterile barrier 232 can further include one or more frame support bosses 228 and 238.

In the embodiment of the sterile barrier 232 shown in FIG. 3G, a width x₁ can be 14 in, about 14 in, between 12 in and 16 in, between 10 in and 18 in, or any other suitable width. In the embodiment of the sterile barrier 232 shown in FIG. 3H, the width x₁ can be 15 in, about 15 in, between 13 in and 17 in, between 11 in and 19 in, or any other suitable width. A height y₁ of the support surface 204 can be 0.125 in, about 0.125 in, between 0.1 and 0.15 in, or any other suitable height. In some embodiments, the support surface 204 can be recessed from a top surface 233 of the sterile barrier 232. A height y₂ between a bottom of the support surface 204 and the top surface 233 can be 0.5 in, about 0.5 in, between 0.25 in and 0.75 in, or any other suitable height. A width x₂ from a lateral edge of the channel 205 to a lateral edge of the channel 207 can be 5 in, about 5 in, between 4 in and 6 in, or any other suitable width. A width x₃ of the support surface 204 can be 4 in, about 4 in, between 3 in and 5 in, or any other suitable width. A height y₃ of the channel 206 and/or channel 212 can be 1.5 in, about 1.5 in, between 1 in and 2 in, or any other suitable height. A width x₄ of the channel 206 and/or channel 212 can be 3 in, about 3 in, between 2 in and 4 in, or any other suitable width. The channel 206 and/or channel 212 can be defined by an arc angle α of 90°, about 90°, between 80° and 100°, or any other suitable angle, and a radius of curvature of 0.125 in, about 0.125 in, between 0.1 and 0.15 in, or any other suitable radius of curvature. In certain embodiments, an arc angle α of 90° or about 90° may be used to hold a hub having a rectangular or generally rectangular cross-section. The support surface 204 can be defined by a radius of curvature of 13 in, about 13 in, between 11 in and 15 in, or any other suitable radius of curvature. The channel 205 and/or channel 207 can be defined by a radius of curvature of 0.25 in, about 0.25 in, between 0.15 in and 0.35 in, or any other suitable radius of curvature.

FIGS. 3L and 3M depict example dimensions of a hub 250 that may be used with the sterile barrier 232 as shown in FIGS. 3G-3K. The hub 250 may be any of the hubs described herein. In certain embodiments, the hub 250 can have a width w₁ of 3.75 in, about 3.75 in, between 3.25 in and 4.25 in, or any other suitable width. The hub 250 can have a height h1 of 1.5 in, about 1.5 in, between 1.25 in and 1.75 in, or any other suitable height. Alternatively, the hub 250 can have a height h₂ of 2 in, about 2 in, between 1.75 in and 2.25 in, or any other suitable height. In some embodiments, the hub 250 can have a length L₁ of 2.5 in, about 2.5 in, between 2 in and 3 in or any other suitable length. Alternatively, the hub 250 can have a length L₂ of 4 in, about 4 in, between 3.25 in and 4.75 in, or any other suitable length.

In some embodiments, a top surface of the support table can include surface features that generally correspond to those of the sterile barrier 232. For example, the support table can include a convex surface configured to correspond to the shape, size, and location of the support surface 204 and/or one or more recesses configured to correspond to the shape, size, and location of the channels 205 and 207.

In alternate embodiments, a planar support surface (for example, support surface 104 of sterile barrier 32) can be positioned at an angle to a horizontal plane to facilitate the draining of fluids. In some embodiments, the sterile barrier and/or support table may be positioned, during part of or the entirety of an interventional procedure, at an angle to a horizontal plane to facilitate the draining of fluids. For example, the sterile barrier and/or support table may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the sterile barrier and/or support table (for example, so that one lateral side of the planar support surface is positioned higher than the other lateral side of the planar support surface, the proximal end is higher than the distal end, or the distal end is higher than the proximal end) to facilitate the drainage of fluids. For example, the drive mechanism may raise or lower one lateral side of the sterile barrier and/or support table, the proximal end of the sterile barrier and/or support table, and/or the distal end of the sterile barrier and/or support table.

In certain embodiments, a support surface (for example, support surface 104 of sterile barrier 32) can be positioned in a vertical configuration instead in the horizontal configuration shown, for example, in FIGS. 3A-3F. For example, the support surface 104 can be positioned at about 90 degrees (or any other suitable angle) from a horizontal plane (e.g., rotated 90 degrees about a long axis of the support surface 104 relative to the embodiment shown in of FIGS. 3A-3F). A vertical configuration may provide for easier interaction with the drive system 18 by a physician. A vertical configuration may also provide for a lower axis of catheter travel closer to a patient without adding standoff height to the drive system 18.

In some embodiments, the drive system 18 may be positioned, during part of or the entirety of an interventional procedure, at an angle to a horizontal plane to facilitate the draining of fluids. For example, the drive system 18 may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the drive system 18 (for example, so that one lateral side of the drive system 18 is positioned higher than the other lateral side of the drive system 18, the proximal end is higher than the distal end, or the distal end is higher than the proximal end) to facilitate the drainage of fluids. For example, the drive mechanism may raise or lower one lateral side of the system 18, the proximal end of the drive system 18, and/or the distal end of the drive system 18. In some embodiments, the drive system 18 may be angled so that it extends at an angle away from axis point 24 (for example, so that the proximal end is higher than the distal end), for example, to allow for clearance of a patient's feet.

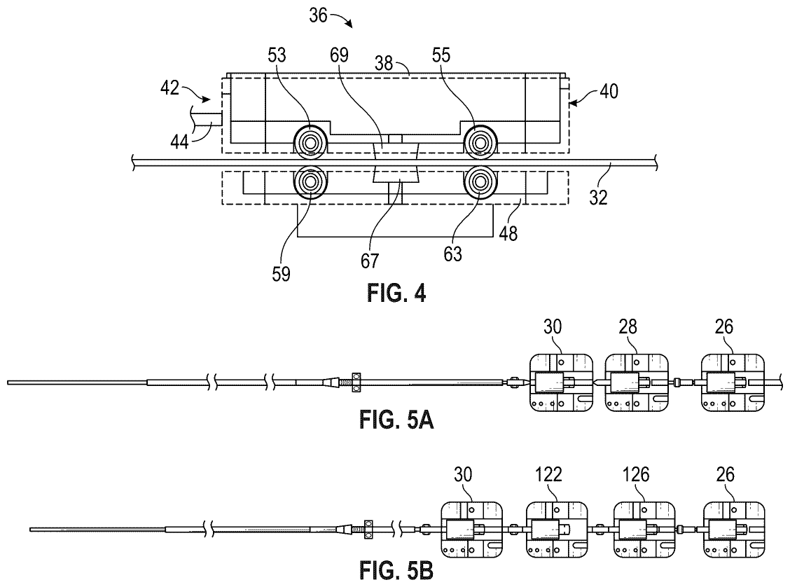

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 or carriage acts as a shuttle by advancing proximally or distally along a track in response to operator instructions or controller manipulations. The hub adapter 48 includes at least one drive magnet 67 configured to couple with a driven magnet 69 carried by the hub 36. This provides a magnetic coupling between the drive magnet 67 and driven magnet 69 through the sterile barrier such that the hub 36 is moved across the top of the sterile barrier 32 in response to movement of the hub adapter 48 outside of the sterile field. Movement of the hub adapter is driven by a drive system carried by the support table and described in additional detail below. The hub adapter may act as a robotic drive for an interventional device coupled thereto.

To reduce friction in the system, the hub 36 may be provided with at least a first roller 53 and a second roller 55 which may be in the form of wheels or rotatable balls or drums. The rollers space the sterile barrier apart from the surface of the driven magnet 69 by at least about 0.02 centimeters (about 0.008 inches) and generally no more than about 0.08 centimeters (about 0.03 inches). In some implementations, the space is within the range of from about 0.03 centimeters (about 0.010 inches) and about 0.041 centimeters (about 0.016 inches). The space between the drive magnet 67 and driven magnet 69 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 48 may similarly be provided with at least a first hub adapter roller 59 and the second hub adapter roller 63, which may be positioned opposite the respective first roller 53 and second roller 55 as illustrated in FIG. 4.

In other embodiments, any of the interventional devices and/or hubs described herein may be coupled to a carriage or hub adapter via a mechanical coupling.

Additional details regarding a magnetic coupling through a sterile barrier can be found in U.S. patent application Ser. No. 18/678,766, entitled MAGNETIC COUPLING THROUGH A STERILE FIELD BARRIER, filed May 30, 2024, the entirety of which is hereby incorporated by reference herein.

Any of the catheters illustrated, for example, in FIG. 5A or 5B, generally include an elongate tubular body extending between a proximal end and a distal functional end. The length and diameter of the tubular body depends upon the desired application. For example, lengths in the area of from about 90 centimeters to about 195 centimeters or more are typical for use in femoral access percutaneous transluminal coronary applications. Intracranial or other applications may call for a different catheter shaft length depending upon the vascular access site.

Certain embodiments of hub assemblies described herein, such as hub assembly (“hub”) 36 shown in FIG. 4, include a housing (e.g., housing 38) for coupling an interventional device thereto, components (e.g., rollers 53 and 55) for directly coupling to and moving along a drive table, and magnet(s) (e.g., magnet 69) for magnetically coupling to a hub adapter across a sterile barrier. A hub (or hub assembly) can refer to a single assembly with a housing, or a hub (or hub assembly) can generally refer to an apparatus having two (or more) subassemblies (e.g., a first subassembly and a second subassembly). In some embodiments of a hub assembly having two subassemblies, a hub can refer to a first subassembly that can be configured to couple to and house an interventional device, and that may be removably attachable to a second subassembly (or mount) configured to magnetically couple to a hub adapter across a sterile barrier and move along a drive table. Such a hub and mount may together form a hub assembly. Such hub assemblies may allow for a hub (first subassembly) to be removed from a mount (second subassembly) which can be advantageous, for example, so that a different hub can be coupled to the same mount or so that the hub may be used separately from the mount (e.g., for a manual procedure).

An arrangement of a hub assembly having a hub that is releasably couplable to mount can allow for replacement of a hub with a different hub having a different interventional device coupled thereto without breaking a magnetic connection with a hub adapter. For example, such an arrangement may allow for a hub coupled to an access catheter to be removed from a mount and replaced with a hub coupled to a procedure catheter without breaking a magnetic connection between active and passive magnetic sides of the coupling of the hub adapter and hub assembly (e.g., between the hub adapter and the mount). In some embodiments, the mount may be a magnetically driven member, an axially driven member, a puck, a slider, a shuttle, or a stage. The robotic control systems described herein can relate to various embodiments of systems that include a hub, or a hub and a mount, regardless of whether they are described in reference to a hub, or a hub and mount, unless explicitly indicated or indicated by context. In some embodiments, a mount may be magnetically coupled to hub adapter across a sterile barrier prior to coupling a hub to the mount, for example, when preparing the drive table for a medical procedure.

As described herein, the hub assemblies can include intravascular devices that can access the vascular system of a patient via at least one artery and/or vein (e.g., the femoral artery) and be driven within the vascular system to perform a vascular procedure.

Additional details regarding a hub assemblies can be found in U.S. patent application Ser. No. 18/986,519, entitled ROBOTIC HUB ASSEMBLY, filed Dec. 18, 2024, which is hereby expressly incorporated by reference in its entirety herein.

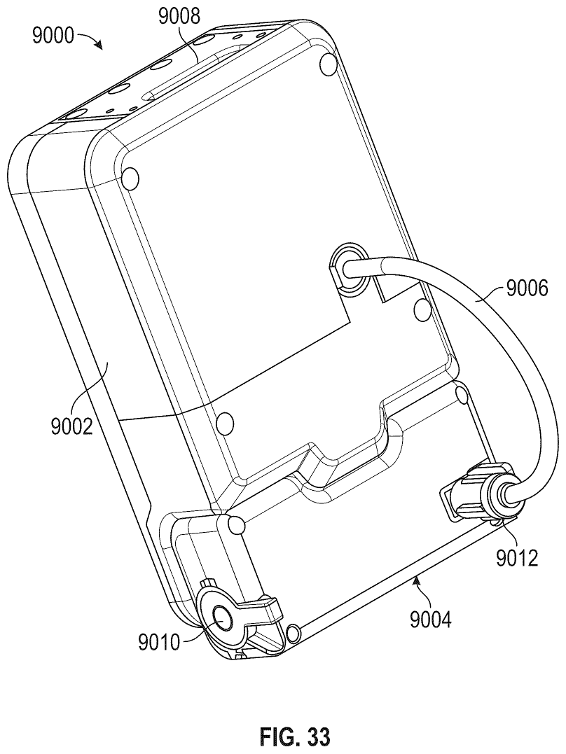

FIG. 33 illustrates a hub assembly 9000. The hub assembly 9000 may include any of the same or similar features and/or functions as the other embodiments of hubs and hub assemblies described herein and vice versa.

In some embodiments, the hub assembly 9000 can include a first subassembly, puck, or mount 9002 and a second subassembly or hub 9004. The mount 9002 can also be referred to as a catheter puck, a hub mount, and/or a first hub member. The mount 9002 can be configured to couple to and move along a drive table. The hub assembly 9000 can be configured to be positioned on a sterile side (e.g., a disposable equipment side) of a sterile barrier.

In some embodiments, the hub 9004 can be referred to as a second hub member. The hub 9004 may include or couple to an interventional device, such as a catheter or guidewire.

As described herein, in certain embodiments an interventional device may be coupled to a fluidics management system (e.g., to receive fluids such as contrast or saline, or for aspiration). In some embodiments, the mount 9002 can be coupled to the fluidics management system. In some embodiments, a fluidics connector 9006 can extend between and fluidly couple the mount 9002 and the hub 9004.

The mount 9002 can further include a first housing. The first housing can define one or more openings 9008 and a plurality of internal components. The first housing can form an outer shell to protect the internal components of the mount 9002. The first housing can include at least one side shaped and/or dimensioned (e.g., having a contour) for receiving the hub 9004.

The one or more openings 9008 can provide access for fluidics and/or electrical connections into the mount 9002. In some embodiments, a contrast tube, a saline tube, and/or an aspiration tube may extend through the one or more openings 9008 into the mount 9002. Additionally, in some embodiments, a power line may extend through the one or more openings 9008 to provide electrical power into the mount 9002. The mount 9002 can be configured to receive an input from one or more active torque elements of an active torque subsystem. In some embodiments, the inputs from the one of more active torque elements may be a magnetic rotary force as described herein. The mount 9002 can be configured to transmit one or more outputs to the hub 9004. In some embodiments, the mount 9002 may transform one or more rotary inputs of the one or more active torque elements into corresponding linear and/or rotary outputs. In some embodiments, the mount 9002 may be configured to translate linearly along a drive table (e.g., in response to linear movement of hub adapter within the drive table due to a magnetic coupling between mount 9002 and the hub adapter).

The hub 9004 can further include a second housing. The hub 9004 can include a lumen 9010 for receiving an interventional device therein. The hub 9004 can include a luer 9012. The hub 9004 can further include a plurality of internal components described in greater detail below. The second housing can form an outer shell to protect the internal components of the hub 9004. In some embodiments, the second housing may include at least one side shaped and/or dimensioned (e.g., having a contour) to correspond to shape of the first housing. For example, the contour of the second housing can correspond to the contour of the first housing of the mount 9002. The hub 9004 can be configured to receive one or more inputs from the mount 9002. The hub 9004 can be configured to transmit one or more outputs. In some embodiments, the hub 9004 may transform the outputs of the mount 9002 into corresponding linear and/or rotary motion of components within or coupled to the hub 9004 (e.g., the interventional device coupled to the hub 9004 and/or one or more fluidics components).

The fluidics connector 9006 can be a tubular body defining an interior lumen extending from one end of the fluidics connector 9006 to a second end of the fluidics connector 9006. In some embodiments, the fluidics connector 9006 may be configured to transport fluids between the mount 9002 and the hub 9004. For example, the fluidics connector 9006 may facilitate the flow of contrast, saline, bodily fluids, and/or air between the mount 9002 and the hub 9004. The fluidics connector 9006 can transport fluids from the mount 9002 to the hub 9004, or vice versa. The fluidics connector 9006 may form an airtight seal.

The hub 9004 may be removably coupled to the mount 9002. In some embodiments, the hub 9004 can be mounted to a mounting element defined by the mount 9002. The fluidics connector 9006 may be coupled to both the mount 9002 and the hub 9004. In some embodiments, the hub 9004 may be in fluid communication with the mount 9002 via the fluidics connector 9006. Accordingly, fluids may be transferred between the mount 9002 and the hub 9004 via the fluidics connector 9006.

Additional details for the fluidics system are described in U.S. patent application Ser. No. 18/666,217, entitled FLUIDICS CONTROL SYSTEM FOR MULTI CATHETER STACK, filed May 16, 2024, the entirety of which is hereby incorporated by reference herein.

Any of a variety of sensors may be provided on any of the catheters, hubs, carriages, or table, depending upon the desired data. For example, in some implementations, it may be desirable to measure axial tension or compression force applied to the catheter such as along a force sensing zone.

It may also be desirable to measure elastic forces across the magnetic coupling between the hub and corresponding carriage, using the natural springiness (compliance) of the magnetic coupling to measure the force applied to the hub. The magnetic coupling between the hubs and carriages creates a spring. When a force is applied to the hub, the hub will move a small amount relative to the carriage. In robotics, this is called a series elastic actuator. This property can be used to measure the force applied from the carriage to the hub. To measure the force, the relative distance between the hub and the carriage is determined and characterize some effective spring constant between the two components.

The relative distance could be measured in multiple different ways. One method for measuring the relative distance between the hub and carriage is a magnetic sensor (e.g., a Hall effect Sensor between hub and carriage). A magnet is mounted to either the hub or carriage, and a corresponding magnetic sensor is mounted on the other device (carriage or hub). The magnetic sensor might be a hall effect sensor, a magnetoresistive sensor, or another type of magnetic field sensor. Generally, multiple sensors may be used to increase the reliability of the measurement. This reduces noise and reduces interference from external magnetic fields.

Other non-contact distance sensors can also be used. These include optical sensors, inductance sensors, and capacitance sensors. Optical sensors would preferably be configured in a manner that avoids accumulation of blood or other fluid in the interface between the hubs and carriages. In some implementations, wireless (i.e., inductive) power may be used to translate movement and/or transfer information across the sterile barrier between a drive carriage and a hub, for example.

The magnetic coupling between the hub and the carriage has a shear or axial break away threshold which may be about 300 grams or 1000 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three-dimensional configuration of the catheter or guidewire during and/or following transvascular placement. Shape sensing fiber optics such as an array of FBG fibers to sense the shape of catheters and guidewires. By using multiple force sensing fibers that are a known distance from each other, the shape along the length of the catheter/guidewire can be determined.

A resistive strain gauge may be integrated into the body of the catheter or guidewire to measure force or torque. Such as at the distal tip and/or proximal end of the device.

Measurements of force and/or torque applied to the catheter or guidewire shafts can be used to determine applied force and/or torque above a safety threshold. When an applied force and/or torque exceeds a safety threshold, a warning may be provided to a user. Applied force and/or torque measurements may also be used to provide feedback related to better catheter manipulation and control. Applied force and/or torque measurements may also be used with processed fluoroscopic imaging information to determine or characterize distal tip motion.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table would have a linear array of corresponding magnetic sensors going the entire length of the table. A processor can be configured to determine the location of the magnet along the length of the linear sensor array, and display axial position information to the physician.

The foregoing may alternatively be accomplished using a non-contact inductive sensor to directly measure the position of the hubs through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

The location of the catheters and guidewires within the anatomy may also be determined by processing the fluoroscopic image with machine vision, such as to determine the distal tip position, distal tip orientation, and/or guidewire shape. Comparing distal tip position or movement or lack thereof to commanded or actual proximal catheter or guidewire movement at the hub, may be used to detect a loss of relative motion, which may be indicative of a device shaft buckling, prolapse, kinking, or a similar outcome (for example, along the device shaft length inside the body (e.g., in the aorta) or outside the body between hubs. The processing may be done in real time to provide position/orientation data at up to 30 Hertz, although this technique would only provide data while the fluoroscopic imaging is turned on. In some embodiments, machine vision algorithms can be used to generate and suggest optimal catheter manipulations to access or reach anatomical landmarks, similar to driver assist. The machine vision algorithms may utilize data to automatically drive the catheters depending on the anatomy presented by fluoroscopy.

Proximal torque applied to the catheter or guidewire shaft may be determined using a dual encoder torque sensor.

Confirming the absence of bubbles in fluid lines may also be accomplished using bubble sensors, particularly where the physician is remote from the patient. This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

It may additionally be desirable for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump.

In an implementation configured for remote operation, any of a variety of sensors may be provided to detect clot passing through the aspiration line and/or trapped in the filter, such as an optical sensor, pressure sensor, flow rate sensor, ultrasound sensor or others known in the art.

The foregoing represents certain specific implementations of a drive table and associated components and catheters. A wide variety of different drive table constructions can be made, for supporting and axially advancing and retracting two or three or four or more drive magnet assemblies to robotically drive interventional devices, fluid elements, and electrical umbilical elements for communicating electrical signals and fluids to the catheter hubs, as will be appreciated by those of skill in the art in view of the disclosure herein. Additional details may be found in U.S. patent application Ser. No. 17/527,393, entitled CATHETER DRIVE SYSTEM FOR SUPRA-AORTIC ACCESS, filed Nov. 16, 2021, the entirety of which is hereby incorporated by reference herein. Additional details may be found in U.S. patent application Ser. No. 18/060,935, entitled “METHOD OF PRIMING AN INTERVENTIONAL DEVICE ASSEMBLY,” filed Dec. 1, 2022, the entirety of which is hereby expressly incorporated by reference herein.

While the foregoing describes robotically driven interventional devices and manually driven interventional devices, the devices may be manually driven, robotically driven, or a combination of both manually and robotically driven interventional devices, as will be appreciated by those of skill in the art in view of the disclosure herein.

In a manual catheter procedure, a physician often stands to a patient's right side and inserts interventional devices from the physician's right to the physician's left when facing the patient. Certain embodiments of robotic control mechanisms described herein may be configured to mimic the movements a physician makes in a manual catheter procedure. For example, certain embodiments of robotic control mechanisms described herein include controls that are operated by left/right motion from the perspective of a user (e.g., a physician) operating the control to command insertion/withdrawal of an interventional device. Certain embodiments of robotic control mechanisms described herein include controls that are operated by rolling or rotational motion from the perspective of a user (e.g., a physician) operating the control to command roll or rotation of an interventional device.

FIG. 6 illustrates a side elevational schematic view of a multi catheter interventional device assembly 2900 for combined supra-aortic access and/or neurovascular site access and procedure (e.g., aspiration), as described herein. The multi catheter assembly 2900 may be configured for either a manual or a robotic procedure.

The interventional device assembly 2900 includes an insert or access catheter 2902, a procedure catheter 2904, and a guide catheter 2906. Other components are possible including, but not limited to, one or more guidewires (e.g., optional guidewire 2907), one or more guide catheters, an access sheath and/or one or more other procedure catheters and/or associated catheter (control) hubs. In some embodiments, the assembly 2900 may also be configured with an optional deflection control 2908 for controlling deflection of one or more catheters of assembly 2900.

In operation, the multi-catheter assembly 2900 may be used without having to exchange hub components. For example, in the two stage procedure disclosed previously, a first stage for achieving supra-aortic access, includes mounting an access catheter, guide catheter and guidewire to the support table. Upon gaining supra aortic access, the access catheter and guidewire were typically removed from the guide catheter. Then, a second catheter assembly is introduced through the guide catheter after attaching a new guidewire hub and a procedure catheter hub to the corresponding drive carriage on the support table.

The devices of the multi catheter assembly 2900 may be operated using a control mechanism (e.g., a handheld controller, a user interface, etc.) as described in further detail herein. The control mechanism may be operated to cause independent or simultaneous axial translation of the interventional devices of the multi catheter assembly 2900 (e.g., along a drive surface). The control mechanism may be operated to cause independent or simultaneous rotational movement of one or more of the interventional devices of the multi catheter assembly 2900.

The single multi catheter assembly 2900 of FIG. 6 is configured to be operated without having to remove hubs and catheters and without the addition of additional assemblies and/or hubs. Thus, the multicomponent access and procedure configuration of assembly 2900 may utilize a guidewire 2907 manufactured to function as an access guidewire and a navigation guidewire to allow for sufficient access and support, and navigation to the particular distal treatment site. In a non-limiting example configured for robotic implementation, a catheter assembly may include a guidewire hub (e.g., guidewire hub 2909 or guidewire hub 26 positioned on a drive table and to the right of catheter 2902), an insert or access catheter hub 2910, a procedure catheter hub 2912, a guide catheter hub 2914 and corresponding catheters. In certain embodiments, one or more of the hubs may include or be coupled to a hemostasis valve (e.g., a rotating hemostasis valve) to accommodate introduction of interventional devices therethrough. In some embodiments, any of the control mechanisms described herein can include at least one control for opening and closing a hemostasis valve.

Additional details regarding hemostasis valves are included in U.S. patent application Ser. No. 17/879,614, entitled MULTI CATHETER SYSTEM WITH INTEGRATED FLUIDICS MANAGEMENT, filed Aug. 2, 2022, which is hereby expressly incorporated by reference in its entirety herein.

One or more of interventional device and hub combinations may further include fluidics connections for coupling to fluid sources and/or vacuum sources. For example, each of the insert or access catheter 2902, the procedure catheter 2904, and the guide catheter 2906 may be in fluid communication with a saline source, a contrast source, and/or a vacuum source. In some embodiments, any of the control mechanisms described herein can include at least one control for initiating and/or terminating the introduction of fluids to one or more of the catheters and/or aspiration of fluids from one or more of the catheters. For example, any of the control mechanisms described herein can include at least one control for opening and/or closing one or more valves to initiate the introduction of fluids to one or more of the catheters and/or aspiration of fluids from one or more of the catheters. For example, any of the control mechanisms described herein can be used to control various components (e.g., manifold valves, pumps, hemostatic valves, hubs, and/or catheters) of a fluidics systems as described in U.S. patent application Ser. No. 17/879,614, entitled Multi Catheter System With Integrated Fluidics Management, filed Aug. 2, 2022, the entirety of which is hereby incorporated by reference herein.

In some embodiments, the control mechanisms described herein may allow a user to simultaneously control movement of a catheter (e.g., axial and/or rotational movement) and a fluidics system (e.g., for introduction of fluids and/or aspiration).

In some implementations, other control operations beyond translational movement and rotational movement may be carried out using any of the controls described herein. For example, the controls may be configured to drive a shape change and/or stiffness change of a corresponding interventional device. Controls may be toggled between different operating modes. For example, controls may be toggled between movement driven by acceleration and velocity to movement that reflects actual linear displacement or rotation.

In some implementations, the control mechanisms may be provided with a visual display or other indicator of the relative positions of the controls which may correspond the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

The systems described herein may compare an actual fluoroscopic image position to an input displacement from the controller. A static fluoroscopic image of the patient may be captured in which the patient's vasculature is indexed relative to bony landmarks or one or more implanted soft tissue fiducial markers. Then a real time fluoroscopic image may be displayed as an overlay, aligned with the static image by registration of the fiducial markers. Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

Additional details regarding a controller for a robotic surgical system can be found in U.S. patent application Ser. No. 18/784,630, entitled SYSTEM FOR REMOTE MEDICAL PROCEDURE, filed Jul. 25, 2024, the entirety of which is hereby incorporated by reference herein.

Once access above the aortic arch has been achieved, the insert or access catheter 2902 (associated with insert catheter hub 2910) may be parked in the vicinity of a carotid artery ostia and the remainder or a subset of the catheter assembly may be guided more distally toward a particular site (e.g., a clot site, a surgical site, a procedure site, etc.).

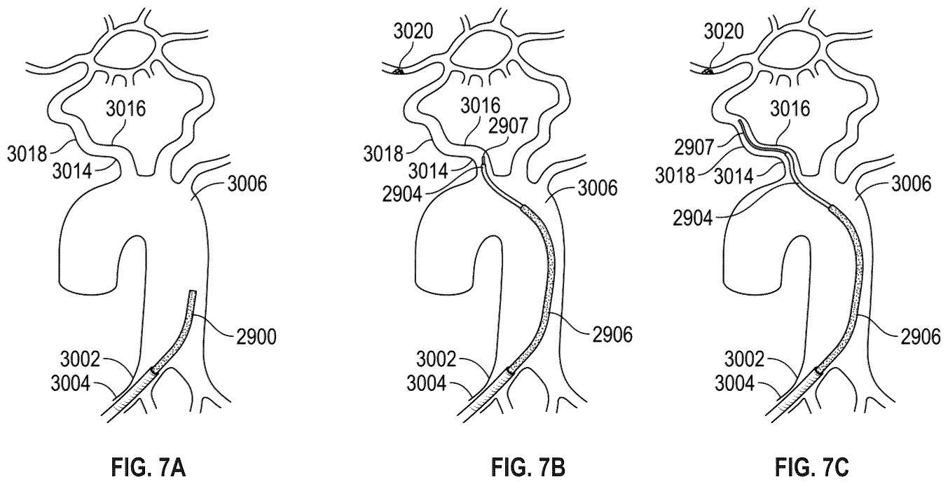

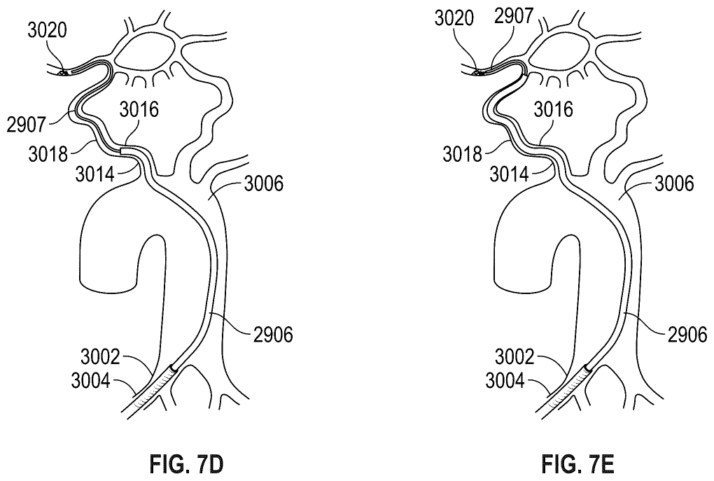

In some embodiments, other smaller procedure catheters may also be added and used at the site. As used herein for catheter assembly 2900, in a robotic configuration of assembly 2900, the catheter 2906 may function as a guide catheter. The catheter 2904 may function as a procedure (e.g., aspiration) catheter. In some embodiments, the catheter 2906 may function to perform aspiration in addition to functioning as a guide catheter, either instead of or in addition to the catheter 2904. The access catheter 2902 may have a distal deflection zone and can function to access a desired ostium. One of skill in the art will appreciate from FIGS. 7A-7E that either manual manipulation or robotic manipulation of the multi catheter stack are contemplated herein.

In some embodiments, the catheter assembly 2900 (or other combined catheter assemblies described herein) may be driven as a unit to a location. However, each catheter (or guidewire) component may instead be operated and driven independent of one another to the same or different locations.

In a non-limiting example, the catheter assembly 2900 may be used for a diagnostic angiogram procedure. In some embodiments, the assembly 2900 may include only the guidewire 2907 and access catheter 2902 (in the form of a diagnostic angiographic catheter) for performing the diagnostic angiogram procedure or only the guidewire 2907 and the access catheter 2902 may be utilized during the procedure. Alternatively, the guide catheter 2906 and procedure catheter 2904 may be retracted proximally to expose the distal end of the access catheter 2902 (e.g., a few centimeters of the distal end of the access catheter) to perform the diagnostic angiography.

As shown in FIG. 6, the guide catheter 2906, procedure catheter 2904, access catheter 2902, and guidewire 2907 can be arranged concentrically. In certain embodiments, the guide catheter 2906 may be a ‘large bore’ guide catheter or access catheter having an inner diameter of at least about 0.075 or at least about 0.080 inches in diameter. The procedure catheter 2904 may be an aspiration catheter having an inner diameter within the range of from about 0.060 to about 0.075 inches. The access catheter 2902 may be a steerable catheter with a deflectable distal tip, having an inner diameter within the range of from about 0.025 to about 0.050 inches. The guidewire 2907 may have an outer diameter within the range of from about 0.014 to about 0.020 inches. In one example, the guide catheter 2906 may have an inner diameter of about 0.088 inches, the procedure catheter 2904 about 0.071 inches, the access catheter 2902 about 0.035 inches, and the guidewire 2907 may have an outer diameter of about 0.018 inches.

FIGS. 7A-7E depict an example sequence of steps of introducing a multi-catheter assembly configured to achieve access all the way to the clot, either manually or robotically. FIGS. 7A-7E may be described using the interventional device assembly of FIG. 6. Other combinations of catheters may be substituted for the interventional device assembly, as will be appreciated by those of skill in the art in view of the disclosure herein.

Referring to FIG. 7A, the three catheter interventional device assembly 2900 is shown driven through an introducer sheath 3002, up through the iliac artery 3004 and into the descending aorta. Next, the access catheter 2902, the procedure catheter 2904 (e.g., 0.071 inch) and the guide catheter 2906 (e.g., 0.088 inch) are tracked up to the aortic arch 3006, as shown in FIG. 7B. Here, the distal end of the guide catheter 2906 may be parked below the aortic arch 3006 and the procedure catheter 2904, access catheter 2902 (positioned within the procedure catheter 2904 and not visible in FIG. 7B), and a guidewire 2907 can be driven into the ostium (e.g., simultaneously or separately). In some embodiments, the access catheter 2902 is advanced out of the procedure catheter 2904 and the guide catheter 2906 to engage the ostium first. After the distal end of the access catheter 2902 is positioned within the desired ostium, the guidewire 2907 can be advanced distally into the ostium to secure access. After the access catheter 2902 and guidewire 2907 are positioned within the desired ostium, the procedure catheter 2904 and/or guide catheter 2906 can be advanced into the ostium (and, in some embodiments, beyond), while using the support of the access catheter 2902 and/or guidewire 2907 to maneuver through the aorta and into the ostium. In the embodiment shown in FIG. 7B, the procedure catheter 2904 has been advanced into the ostium while the guide catheter 2906 has remained parked below the aortic arch 3006.

Referring to FIG. 7C, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 7D, the guide catheter 2906 and the procedure catheter 2904 (positioned within the guide catheter 2906 and not visible in FIG. 7D) are both advanced (e.g., simultaneously or sequentially) over the guidewire 2907 and over the insert or access catheter 2902 (positioned within the procedure catheter 2904 and not visible in FIG. 7D) while the access catheter 2902 remains at the ostium for support. The guidewire 2907 may be further advanced past the petrous segment 3018 to the site of the clot 3020, such as the M1 segment.

Referring to FIG. 7E, the guide catheter 2906 and the procedure catheter 2904 (positioned within the guide catheter 2906 and not visible in FIG. 7E) are advanced (e.g., simultaneously or sequentially) to position the distal tip of the procedure catheter 2904 at the procedure site, for example on the face of the clot 3020. The guidewire 2907 and access catheter 2902 (positioned within the procedure catheter 2904 and not visible in FIG. 7E) are removed, and aspiration of the clot 3020 commences through the procedure catheter 2904. That is, the guidewire 2907 and the access catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procedure catheter 2904 may be removed before removing the guide catheter 2906.

In some embodiments, aspiration may be performed through two catheters (e.g., the procedure catheter 2904 and the guide catheter 2906) simultaneously. For example, during a thrombectomy procedure, the clot 3020 may become engaged with or corked at a distal end of the procedure catheter 2904 (or another inner catheter). In such cases, it may be necessary to remove the procedure catheter 2904 (or other inner catheter) from the vasculature of the patient to remove the clot 3020. As the procedure catheter 2904 (or other inner catheter) is retracted from the guide catheter 2906 (or other outer catheter), debris from the stuck clot 3020 can dislodge. In such embodiments, application of vacuum at both the procedure catheter 2904 (or other inner catheter) and the guide catheter 2906 (or other outer catheter) can beneficially prevent the debris from flowing distally into the vasculature of the patient by aspirating the debris and thus reducing the risk of embolization.

In some embodiments, the clot 3020 may become engaged with or corked at the distal end of the guide catheter 2906 (or other outer catheter). Vacuum through the guide catheter 2906 (or other outer catheter) may prevent dislodgement of the clot 3020 from the guide catheter 2906 (or other outer catheter). In some instances, it may not be readily apparent if portions of a clot are engaged with the guide catheter 2906 (or other outer catheter) or the procedure catheter 2904 (or other inner catheter). In such instances, vacuum through both the guide catheter 2906 (or other outer catheter) and the procedure catheter 2904 (or other inner catheter) may prevent debris from flowing distally into the vasculature of the patient by aspirating the debris and thus reducing the risk of embolization.

The catheter assembly 2900 may be used to perform a neurovascular procedure, as described in FIGS. 7A-7E. For example, the neurovascular procedure may be a neurovascular thrombectomy. The steps of the procedure may include providing an assembly that includes at least a guidewire, an access catheter, a guide catheter, and a procedure catheter. For example, the catheter assembly 2900 includes a guidewire 2907, an access (e.g., insert) catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter, a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system, and driving the assembly to achieve supra-aortic access. The steps may further include driving a subset of the assembly to a neurovascular site, and performing the neurovascular procedure using a subset of the assembly. The subset of the assembly may include the guidewire, the guide catheter, and the procedure catheter.

Each of the guidewire 2907, the access catheter 2902, the guide catheter 2906, and the procedure catheter 2904 is configured to be adjusted by a respective hub. For example, the guidewire 2907 may include (or be coupled to) a hub installed on one of the tray assemblies described herein. Similarly, the access catheter 2902 may be coupled to catheter hub 2910. The guide catheter 2906 may be coupled to the guide catheter hub 2914. The procedure catheter 2904 may be coupled to the procedure catheter hub 2912.

In general coupling of the assembly may include magnetically coupling a first hub 2909 on the guidewire 2907 to a first drive magnet, magnetically coupling a second hub 2910 on the access catheter 2902 to a second drive magnet, magnetically coupling a third hub 2912 on the procedure catheter 2904 to a third drive magnet, and magnetically coupling a fourth hub 2914 on the guide catheter 2906 to a fourth drive magnet. In general, the first drive magnet, the second drive magnet, the third drive magnet, and the fourth drive magnet are each independently movably carried by a drive table, as described with respect to tray assemblies and controls described herein. In some embodiments, the first drive magnet, the second drive magnet, the third drive magnet, and the fourth drive magnet are coupled (e.g., to their respective catheter hubs) through a sterile barrier (e.g., a sterile and fluid barrier) and independently movably carried by a drive table having a plurality of driven magnets. In some embodiments, two or more drive magnets can be tethered or otherwise coupled together to move as a unit in response to commands from a single controller tethered or otherwise coupled to one of the drive magnets.

In some implementations, the steps of performing the neurovascular procedure may include driving the assembly in response to movement of each of the hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a driven magnet carried by the respective hub. This provides a magnetic coupling between the drive magnet and driven magnet through the sterile barrier such that the respective hub is moved across the top of the sterile barrier in response to movement of the hub adapter outside of the sterile field (as described in detail in FIG. 4). Movement of the hub adapter is driven by a drive system carried by the support table in which the guidewire hub 2909, the guide catheter hub 2914, the procedure catheter hub 2912, and the access catheter hub 2910 are installed upon.

The steps may further include driving a subset of the assembly in response to movement of each of the hub adapters along the support table until the subset of the assembly is positioned to perform a neurovascular procedure at a neurovascular treatment site. The subset of the assembly may include the guidewire 2907, the guide catheter 2906, and the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the catheter assembly 2900 may be part of a robotic control system for achieving supra-aortic access and neurovascular treatment site access, as described in FIGS. 7A-7E. In some embodiments, the catheter assembly 2900 may be part of a manual control system for achieving supra-aortic access and neurovascular treatment site access. In some embodiments, the catheter assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, access catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an access catheter hub 2910 configured to adjust axial and rotational movement of an access catheter 2902. The robotic control system may also include a guide catheter hub 2914 configured to control axial movement of a guide catheter 2906. The robotic control system may also include a procedure catheter hub 2912 configured to adjust an axial position and a rotational position of a procedure catheter 2904.

In some embodiments, the procedure catheter hub 2912 is further configured to laterally deflect a distal deflection zone of the procedure catheter 2904.

In some embodiments, the guidewire hub 2909 is configured to couple to a guidewire hub adapter by magnetically coupling the guidewire hub to a first drive magnet. The access catheter hub 2910 is configured to couple to an access catheter hub adapter by magnetically coupling the access catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes
a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail with respect to FIG. 4 above.

In some embodiments, the robotic control system includes a second driven magnet on the access catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

### Control Mechanisms and User Interface

To facilitate control of any of the control mechanisms for interventional devices described herein, a user interface can be implemented along with the control mechanism. For instance, a user interface could beneficially allow clinicians to, among other things, visualize the position and movement of the interventional devices as the clinician manipulates the interventional devices using a control mechanism. This can also beneficially allow clinicians to observe the actual movement of the interventional devices as opposed to simply estimating the movement and position of the interventional devices inside the patient.

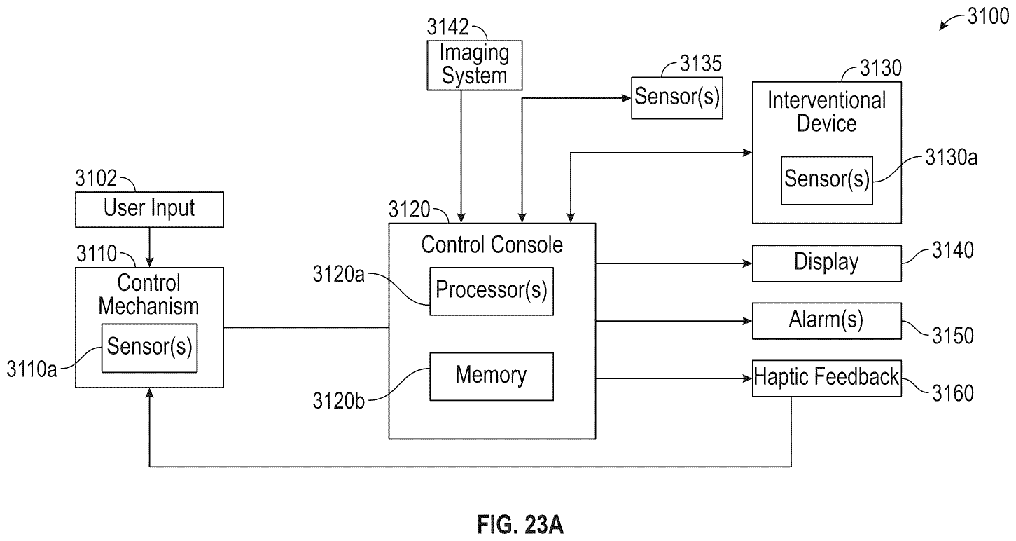

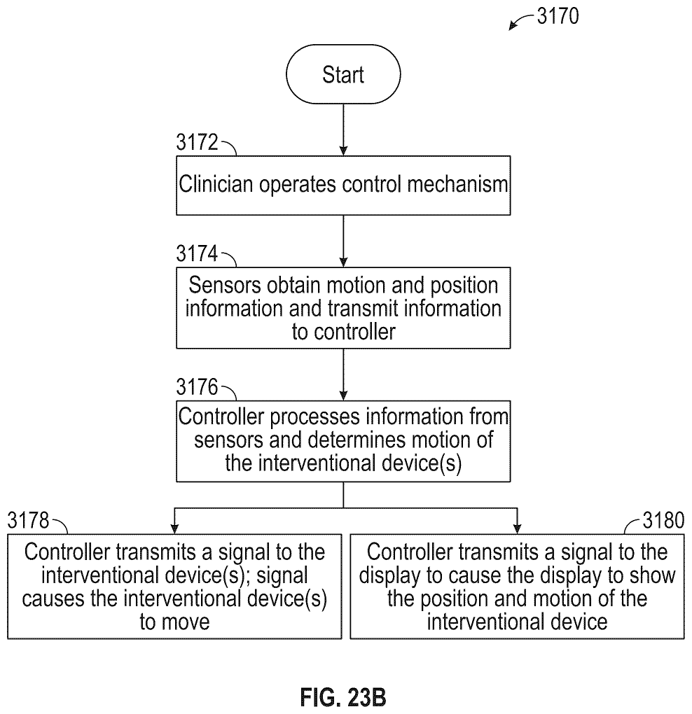

FIG. 23A illustrates a simplified block diagram of a medical device operation environment 3100, according to an embodiment of the disclosure. The operation environment 3100 can include a controller or control mechanism 3110, a control system 3120, at least one interventional device 3130, and a display 3140. The control system 3120 can be a control station or control console, or be in communication with a control station or control console. The control system 3120 can be in communication with the control mechanism 3110, the at least one interventional device 3130, and the display 3140. Any of the components of the operation environment 3100 can be in wired or wireless communication through, for example, a connected cloud server, with each other. For example, as the sensors 3110a of the control mechanism 3110 can obtain information about the user's operation of the control mechanism 3110. For example, the sensors 3110a can detect how the control mechanism 3110 is manipulated by the clinician (e.g., can detect if the clinician moves a joystick, in what direction the joystick moves, if a button is pressed, etc.). The sensors 3110a can include any of the sensors described herein. The information obtained by the sensors 3110a can be transmitted to the control system 3120 through a cable or through a wireless connection.

The control system 3120 can receive information from the sensors 3110a of the control mechanism 3110. The control system 3120 can include a circuit having a processor 3120a, input ports for receiving the information from the sensors 3110a, a memory 3120b, and software stored in the memory 3120b in which can be executed by the processor 3120a. The processor 3120a can include digital signal processors, application specific integrated circuit (ASIC), a field programmable gate array (FPGA) or other programmable logic device, discrete gate or transistor logic, discrete hardware components, or any combination thereof to perform the functions described herein. The processor 3120a can include electrical circuitry or digital logic circuitry configured to process computer-executable instructions. In some cases, the processor 3120a can include an FPGA or other programmable device that performs logic operations without processing computer-executable instructions. The processor 3120a can also be implemented as a combination of computing devices, for example, a combination of a DSP and a microprocessor, a plurality of microprocessors, one or more microprocessors in conjunction with a DSP core, or any other such configuration.

The control system 3120 can process the signals from the sensors 3110a and utilize the display 3140 to show how the control mechanism 3110 is being manipulated and/or show the position of the interventional devices 3130 with respect to each other. The sensor information can include information from the sensors of any of the control mechanisms disclosed herein. The sensors 3110a of the control mechanism 3110 can detect the user input 3102 on the control mechanism 3110.

The control system 3120 can process the signals received from the sensors 3110a of the control mechanism 3110 and send signals to the interventional device. The signals to the interventional device can include instructions to the interventional device to move according to the user input as detected by the sensors 3110a of the control mechanism 3110. The sensors 3110a of the one or more interventional devices 3130 can detect information relating to the position and movement of the interventional devices 3130. Position and motion information can be transmitted to the control system 3120.

The at least one interventional device 3130 can include sensors 3130a that can detect and/or track movement of the at least one interventional device 3130. For example, the sensors 3130a of the at least one interventional device 3130 can include one or more linear positions sensors and/or rotation sensors than can detect movement and/or track the position of the interventional device 3130 in real time, and/or any of the sensors described herein (e.g., pressure sensors, optical sensors, force sensors, positioning sensors, temperature sensors, oxygen sensors, and/or Fiber Bragg grating sensors). The sensors 3130a can include one or more of an inertial measurement unit sensor, an accelerometer, a gyroscope, a linear potentiometer, a linear variable differential transformer (LVDT) sensor, an ultrasonic sensor, a laser sensor, a hall effect sensor, a magnet, a magnet resistive element, an encoder, a rotary potentiometer, a rotary variable differential transformer (RVDT) sensor, or a combination thereof to detect axial position, angular position, detect and/or measure linear movement, and/or detect and/or measure rotational movement of each of the interventional devices, hubs, and/or hub adapters.

The sensors 3130a of the at least one interventional device 3130 can monitor the operational status of the at least one interventional device 3130. The sensor information from the interventional device 3130 can be communicated to the control system 3120. Based at least in part on the sensor information received from the interventional device 3130, the control system 3120 can output information relating to the one or more interventional devices 3130 via a display 3140. The display 3140 can include, for example, display 23. In some cases, the display 3140 can be carried on a portable, handheld device or desktop computer, and may be located in the same room as the patient, the same facility as the patient, or in a remote facility.

In some embodiments, the medical device operation environment 3100 can include one or more sensors 3135 configured to detect the coupling of an interventional device to a drive table. In some embodiments, the medical device operation environment 3100 can include one or more sensors 3135 configured to detect the identity of an interventional device coupled to the drive table. For example, the one or more sensors 3135 may detect a type of interventional device (e.g., a guide catheter, a procedure catheter, an access catheter, a guidewire, etc.), dimensions of the interventional device, a serial number of the interventional device, and/or if the interventional device is an authorized interventional device for using with the drive table. For example, in some embodiments, the sensor can read data from an RFID tag, bar code, and/or other machine readable data source of the interventional device to detect interventional device identify information. In some embodiments, a sensor or scanner 3135 for detecting and/or receiving the interventional device identity information can be positioned on or within the drive table, on or within a hub, on or within a hub adapter, on or within an interventional device, or at any other suitable location.

The sensor information from the sensor(s) 3135 can be communicated to the control system 3120. Based at least in part on the sensor information received from the sensor(s) 3135 the control system 3120 can output information relating to the one or more interventional devices 3130 via a display 3140. The display 3140 can include, for example, display 23. In some cases, the display 3140 can be carried on a portable, handheld device or desktop computer, and may be located in the same room as the patient, the same facility as the patient, or in a remote facility. As described further herein, in some embodiments, a control system can output information from the sensors 3135 to a graphical user interface 5400 to associate interventional device identify information with one or more visual representations of interventional devices.

The control system 3120 can communicate with an imaging system 3142. In some cases, the imaging system 3142 can record, capture, and/or transmit fluoroscopic images and/or video. For instance, the imaging system 3142 can include a display for showing a live fluoroscopic video feed of a patient's vasculature and/or the interventional devices. The imaging data, as captured by the imaging system 3142, can be communicated to the control system 3120. The control system 3120 can process the imaging data of the imaging system 3142 and send it to the display 3140. The imaging data can be displayed on the display 3140. In some cases, the control system 3120 can mirror the image feed shown on the display of the imaging system 3142 and show it on a portion of the display 3140 (e.g., on a window). The imaging system 3142 can beneficially allow clinicians to visualize the one or more interventional devices 3130 in the vasculature of a patient on the display 3140 as the interventional devices 3130 are advanced in and/or retracted from the body of the patient.

In some cases, the controller can process the sensor information obtained by the sensors 3110a of the control mechanism 3110 and/or the sensor information obtained by the sensors 3130a of the interventional devices 3130. The control system 3120 can determine, based on the motion and/or the position of the control mechanism 3110 and/or the interventional devices 3130 if the control mechanism 3110 and/or the interventional devices 3130 are operating correctly. For example, the control system 3120 can determine the interventional devices 3130 are not operating properly if movement of the interventional devices 3130 does not correspond to the user input 3102 on the control mechanism 3110. Upon detecting a faulty condition on either the control mechanism 3110 and/or the interventional devices 3130, the control system 3120 can emit an alert 3150. The alert 3150 can provide an indication to the clinician that the control mechanism 3110 and/or the interventional devices 3130 are not operating properly. In some cases, the alert 3150 can be displayed on the display 3140.

The control system 3120 can also process the sensor information from sensors 3110a and/or 3130a and send a signal to the control mechanism 3110. The signal can include haptic feedback 3160 which can cause operation of the control mechanism 3110 to change. For example, the haptic feedback 3160 can cause the components of the control mechanism 3110 to harden or soften, which may require a clinician to exert more force or less force to press or move a component. The haptic feedback 3160 can provide an indication to the clinician about the position of the one or more interventional devices. For example, the components of the control mechanism 3110 may require a clinician to exert more force as a first interventional device moves closer to a second interventional device. The components of the control mechanism 3110 may require a clinician to exert less force as a first interventional device moves away from a second interventional device.

In some embodiments, if the control mechanism 3110 is triggering movement of at least one interventional device 3130 and the sensors 3130a of the at least one interventional device 3130 detect a large force at the tip, the control system 3120 may cause actuators to generate haptic feedback 3160 to indicate to the user to stop or reverse a performed movement, and/or to otherwise cause the components of the control mechanism 3110 to adjust to reduce the force at the tip of the interventional device. In some implementations, haptic feedback 3160 may be generated at the control system 3120 to indicate to the user to reduce or increase a speed of a movement using the control mechanism 3110 or to reverse a movement or perform an alternative movement using the control mechanism 3110. In some implementations, haptic feedback 3160 may provide feedback on a large torsional strain buildup that might precede an abrupt rotation, or a large axial force buildup that may be a prelude to buckling of the catheter. Alternatively or additionally, feedback may be provided by one or more visual indicators (e.g., warning lights), audible indicators, etc.

FIG. 23B illustrates an embodiment of a process 3170 for displaying the position and movement of the interventional devices using a display and causing the interventional device to move according to user-provided input. In some cases, the process can be implemented by any of the control mechanisms, interventional devices, controllers, and displays described herein. In particular, the process 3170 can be implemented by one or more of the components of the medical device operation environment 3100. Advantageously, in certain embodiments, the process 3170 facilitates the generation of position and motion data based on information as obtained from the sensors of the control mechanism.

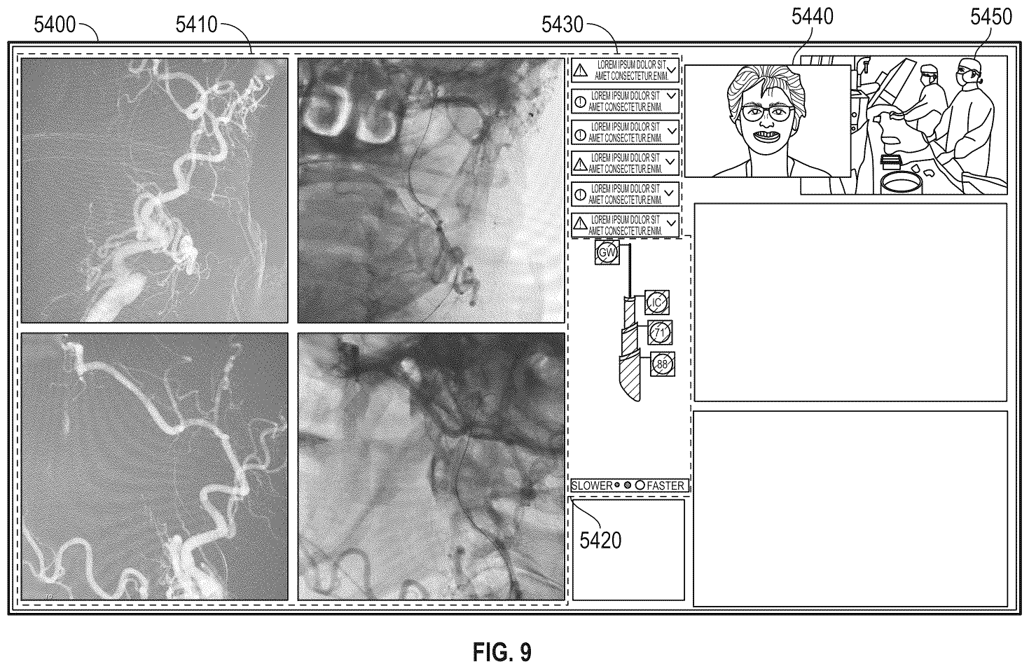

At block 3172, the user operates the control mechanism (e.g., control mechanism 3110 from FIG. 23A). At block 3174, the sensors (sensors 3110a from FIG. 23A) obtain motion and position information from the control mechanism. The information obtained by the sensors of the control mechanism can be transmitted to the controller (e.g., control system 3120 from FIG. 23A). At block 3176 the controller processes the information from the sensors and determines an instruction for the interventional devices (e.g., interventional device 3130 from FIG. 23A). When transmitted to the interventional devices at block 3178, the instruction can cause the interventional device to move according to the user-input on the control mechanism as detected by the sensors. At block 3180, the controller transmits a signal to a display (e.g., display 3140 of FIG. 23A) which causes the display to show a graphical representation of the interventional device's motion and position relative to each other. The graphical representation can include some or all of the elements described below in relation to FIGS. 9-21, 35-37D, and 39A-39H.

In certain embodiments, a control, such as a joystick, knob, button, etc., can be selectively linked to control a particular hub and/or interventional device, e.g., by a user making a selection (e.g., by actuating one or more other controls of a controller) corresponding to the particular hub and/or interventional device so that movement of the control controls movement of the particular hub and/or interventional device. A user may change the particular hub and/or interventional device linked to the control by making an alternate selection.

In certain embodiments, a control, such as a joystick, knob, button, or any other suitable actuator, can be selectively linked to control a particular combination of two or more hubs and/or interventional devices, e.g., by a user making a selection (e.g., by actuating one or more other controls of a controller) corresponding to the particular combination of hubs and/or interventional devices so that movement of the control controls movement of the particular combination of hubs and/or interventional device. For example, when the control is linked to the particular combination of devices, movement of the control may cause corresponding axial and/or rotational movement of each hub and/or interventional device in the particular combination of hubs and or interventional devices (e.g., simultaneously or successively). A user may change the particular combination of hubs and/or interventional devices linked to the control by making an alternate selection.

In some embodiments, when the control is linked to the particular combination of devices, movements of the control that correspond to axial hub/interventional device motion may cause each of the hubs and/or interventional devices to move axially, but movements of the control that correspond to rotational interventional device motion may cause only a subset of the interventional devices to rotate (e.g., in embodiments in which at least some of the interventional devices do not rotate or rotation is not desired). For example, in some embodiments having a guide catheter, a procedure catheter, an access catheter, and a guidewire, the access catheter and guidewire may be configured to rotate, and the guide catheter and procedure catheter may not be configured to rotate. In such embodiments, for example, movements of a control linked to one or more of the guidewire and the access catheter and linked to one or more of the procedure catheter and the guide catheter that correspond to rotational interventional device motion may cause rotation of only the one or more of the guidewire and the access catheter without causing rotation of the one or more of the procedure catheter and the guide catheter.

In some embodiments, when the control is linked to the particular combination of devices, movements of the control that correspond to rotational interventional device motion may cause each of the interventional devices to rotate, but movements of the control that correspond to axial hub/interventional device motion may cause only a subset of the interventional devices to move axially.

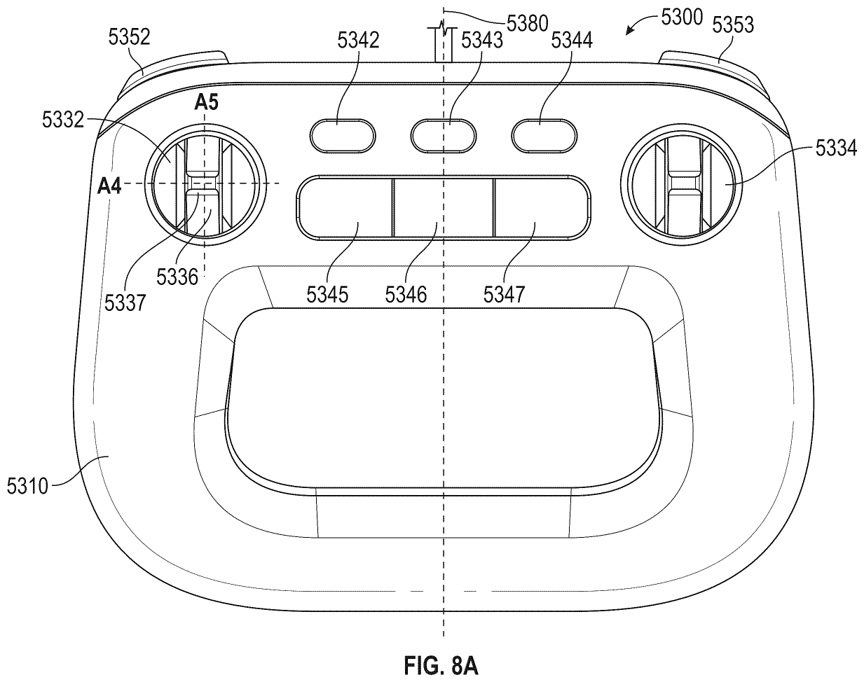

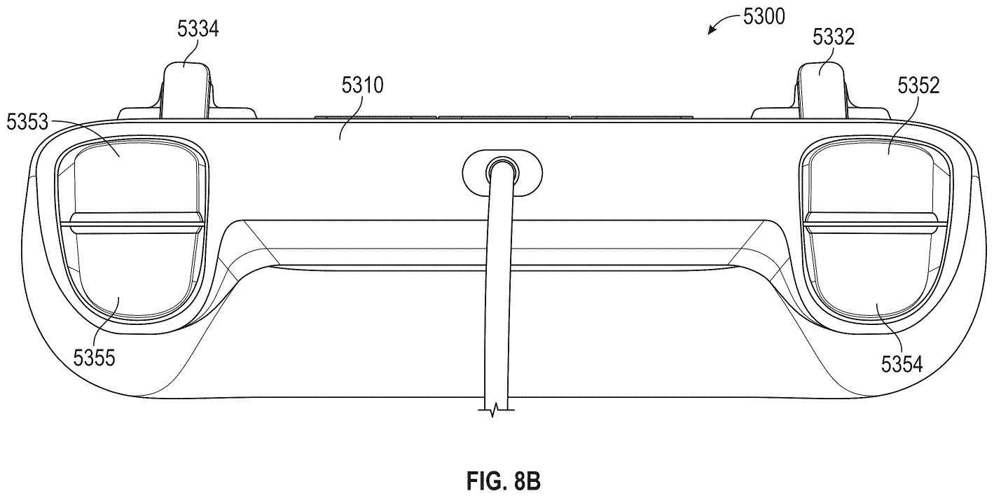

FIGS. 8A-8B illustrate an embodiment of a control mechanism 5300 for manipulating interventional devices driven by (or otherwise associated with) respective hubs. As shown in FIGS. 8A-8B, the control mechanism 5300 can be in the form of a handheld controller. The control mechanism may include any of the same or similar features and/or functions as any of the other control mechanisms described herein.

As shown in FIGS. 8A-8B, the control mechanism 5300 can include a handheld controller 5310 having one or more controls, which may be in the form of buttons and/or joysticks or any other suitable actuators. As shown, the control mechanism 5300 can include a first control or first joystick 5332. The control mechanism 5300 can include a second control or second joystick 5334. The control mechanism can include a plurality of buttons 5342, 5343, 5344, 5345, 5346, 5347, and a plurality of shoulder or bumper buttons 5352, 5353, 5354, 5355.

Each of the first joystick 5332 and the second joystick 5334 may correspond to and drive movement of a hub and/or interventional device. In some cases, each of the first joystick 5332 and the second joystick 5334 can be assignable to control different hubs and/or interventional devices. For example, the first joystick 5332 can be selectively linked to a first particular hub and/or interventional device, for example, by a user making a selection on the controller 5310 (e.g., by pressing one or more buttons). The second joystick 5334 can be selectively linked to a second particular hub and/or interventional device, for example, by a user making a selection on the controller 5310 (e.g., by pressing one or more buttons). In some embodiments, the first joystick 5332 and/or the second joystick 5334 can be selectively linked to a combination of hubs and/or interventional devices, for example, by a user making a selection on the controller 5310 (e.g., by pressing one or more buttons). For example, the first joystick 5332 and/or the second joystick 5334 can be selectively linked to a combination of two hubs and/or interventional devices, three hubs and/or interventional devices, or four hubs and/or interventional devices to simultaneously control at least certain movements (e.g., axial/linear movement and/or rotational movement) of each hub and/or interventional device in the combination. In certain embodiments, the joystick 5332 or joystick 5334 can correspond to and drive movement of a hub and/or interventional device by driving movement of a hub adapter associated with the interventional device.

The control mechanism 5300 can include one or more drive modes in which the first joystick 5332 and the second joystick 5334 are each linked to, and can control, one or more corresponding hubs and/or interventional devices. In some embodiments, in different drive modes, the first joystick 5332 and/or the second joystick 5334 can be configured to control different hubs and/or interventional devices or different combinations of hubs and/or interventional devices.

In some embodiments, the control mechanism 5300 can toggle between drive modes (e.g., in response to a user input). In some embodiments, in different drive modes, the first joystick 5332 and/or the second joystick 5334 can control different subsets of corresponding hubs and/or interventional devices. In some embodiments, in different drive modes, one or both of the first joystick 5332 and/or the second joystick 5334 can control corresponding hubs and/or interventional devices at different axial and/or rotational velocities.

In some embodiments, the control mechanism 5300 can be selectively configured in a first drive mode, which is also referred to herein as a first operation mode, a first stage mode, and/or an access stage mode. As described in further detail herein, in the first drive mode, the control mechanism 5300 can be configured to control a plurality of hubs and/or interventional devices to drive interventional devices to achieve supra-aortic access.

In some embodiments, the control mechanism 5300 can be selectively configured in a second drive mode, which is also referred to herein as a second operation mode, a second stage, and/or a procedure stage mode. As described in further detail herein, in the second drive mode, the control mechanism 5300 can be configured to control one or more hubs and/or interventional devices to drive the interventional devices to a procedure site (e.g., a neurovascular procedure site, such as a thrombectomy site) after supra-aortic access has been achieved. In some embodiments, in the second drive mode, the control mechanism 5300 can be configured to control the plurality of hubs and/or interventional devices to perform a procedure (e.g., a neurovascular procedure, such as a thrombectomy).

In some embodiments, one or more hubs and/or interventional devices may be driven to retract the interventional devices from the procedure site using the control mechanism 5300 in the second drive mode. In some embodiments, one or more hubs and/or interventional devices may be driven to retract the interventional devices from the procedure site using the control mechanism 5300 in the second drive mode until the interventional devices are withdrawn from the supra-aortic vessels. In some embodiments, after the interventional devices are withdrawn from the supra-aortic vessels, the hub and/or interventional devices can be driven to retract the interventional devices in the first drive mode. In other embodiments, there may be one or more additional drive modes for retracting the interventional devices from the procedure site and/or after withdrawal from the supra-aortic vessels.

In some embodiments, in the first drive mode, the first joystick 5332 can be linked to an interventional device such as a guidewire (e.g., guidewire 27 or guidewire 2907), and the second joystick 5334 can be linked to a combination of corresponding hubs and/or international devices. The combination of corresponding hubs and/or international devices can include a guide catheter (e.g., guide catheter 31 or guide catheter 2906), a procedure catheter (e.g., catheter 29, catheter 120, or catheter 2904), and/or an access catheter (e.g., catheter 124 or catheter 2902). In certain embodiments, the first drive mode may be used to drive the hubs and/or interventional devices to achieve supra-aortic access as described herein.

In some embodiments, in the second drive mode, the first joystick 5332 can be linked to an interventional device such as a guidewire (e.g., guidewire 27 or guidewire 2907), and the second joystick 5334 can be linked to a combination of corresponding hubs and/or international devices including a guide catheter (e.g., guide catheter 31 or guide catheter 2906), and/or procedure catheter (e.g., catheter 29, catheter 120, or catheter 2904). As described herein, in certain embodiments, the second drive mode may be used to drive the hubs and/or interventional devices to navigate the interventional devices to a procedure site and/or perform a procedure.

Although reference is made to the control mechanism 5300 including two drive modes, the control mechanism 5300 can include a single drive mode and or more than two drives modes, with each of the first joystick 5332 and the second joystick 5334 linked to one or more corresponding hubs and/or interventional devices in each drive mode. Additionally, as described herein, a user may change which hubs and/or interventional devices are driven by each of the first joystick 5332 and second joystick 5334 while operating in a particular drive mode, for example, by making a selection using the control mechanism 5300 (e.g., by pressing one or more buttons on the control mechanism 5300).

In certain embodiments, users can toggle between drives modes by making a user input via the control mechanism 5300 (e.g., by pressing one of the buttons of the control mechanism 5300). For example, users can toggle between the first drive mode and the second drive mode by pressing the button 5344. As way of example, if the control mechanism 5300 is in the first drive mode, pressing the button 5344 once can cause the control mechanism to switch to the second drive mode. In other embodiments, a user may press a first button to activate the first drive mode and a second button to activate the second drive mode.

In some embodiments in which a plurality of interventional devices is linked with one of the joysticks 5332 and 5334, movement of the joystick 5332 or 5334 can cause the linked interventional devices to move different axial distances relative to one another and/or at different axial velocities relative to one another.

For example, in some embodiments, it may be desirable for the distal ends of a plurality of interventional devices to be arranged at particular positions relative to one another for a particular step of a surgical procedure. In some embodiments, it may be desirable that the distal ends of the plurality of interventional devices may be arranged at different particular positions relative to one another at a different step of the surgical procedure. In some embodiments, a plurality of interventional devices linked to a single joystick 5332 or 5334 can be driven from a first set of positions to a second set of positions in which at least some of the interventional devices are at different relative positions to one another in comparison to the first set of positions (e.g., by moving the linked interventional devices different distances relative to one another and/or at different axial velocities relative to one another).

For example, in certain embodiments, the distal ends of the guide catheter, access catheter, and procedure catheter may be parallel or almost parallel upon initial insertion into the patient access point (e.g., femoral access point). The guide catheter, access catheter, and procedure catheter may be linked to the joystick 5334 (e.g., in the first drive mode), and can be axially driven by the joystick 5334 to desired positions for achieving supra-aortic access (e.g., desired positions within the aortic arch) in which the relative positions of the guide catheter, access catheter, and procedure catheter are different than the relative positions upon initial insertion. This may be achieved, for example, by driving the guide catheter, access catheter, and procedure catheter at different velocities from their positions at initial insertion to the desired positions for achieving supra-aortic access in response to movement of the joystick 5334. For example, in some embodiments, it may be desirable for the access catheter to extend further distally than the procedure catheter and the guide catheter for achieving supra-aortic access. In some embodiments, it may be desirable for the procedure catheter to extend further distally than the guide catheter for achieving supra-aortic access.

Similarly, after supra-aortic access is achieved, the distal ends of the guide catheter and the procedure catheter may be arranged at a first set of positions. The guide catheter and procedure catheter may be linked to the joystick 5334 (e.g., in the second drive mode), and can be axially driven by the joystick 5334 to a second set of positions for performing a procedure in which the relative positions of the guide catheter and the procedure catheter are different than the relative positions at the first set of positions. This may be achieved, for example, by driving the guide catheter and procedure catheter at different velocities from the first set of positions to the second set of positions.

In certain embodiments in which multiple interventional devices linked to a joystick 5332 or 5334 move at different velocities, movement of the joystick may correspond to a velocity of one of the interventional devices (e.g., a leading or distal most interventional device, or an interventional device intended to have a distal most position at the end of a current procedure step), and the velocities of the other interventional devices relative to movement of the joystick may be reduced proportionally (e.g., to correspond to their intended positions at the end of the current procedure step).

In certain embodiments, the interventional devices linked to a joystick 5332 or 5334 may move at different relative velocities until a desired set of second positions is reached for a particular procedure step, and then may all move at the same relative velocity. In other embodiments, after the desired set of second positions is reached. The interventional devices may move at a different set of relative velocities, for example, to reach a third set of relative positions different from the second set of relative positions.

In some embodiments, different interventional devices can be associated with different axial and/or rotational velocity profiles. The velocity profiles may be stored in one or more parameter files which can be stored in a memory and can be accessed and implemented by a control system. In certain embodiments, a user may be able to customize the velocity profiles associated with one or more interventional devices and/or joysticks. In certain embodiments, the user can create a user profile that includes such customized velocity profiles. The user profile may be stored in a memory and accessed and implemented by the control system.

In some embodiments, users can selectively link at least one of the first joystick 5332 and the second joystick 5334 to a single corresponding hub and/or interventional device, for example, when the control mechanism 5300 is in a drive mode (e.g., first and/or second drive mode). In some such embodiments, only the interventional device temporarily linked to the joystick may move in response to movement of the joystick, and not any other interventional devices assigned to the joystick in the particular drive mode in the absence of a temporary linkage. This can beneficially allow clinicians to operate (e.g., translate) a hub and/or interventional device independently during any stage of a procedure (or operate two hubs and/or interventional devices independently when each of the first joystick 5332 and the second joystick 5334 is linked with only a single corresponding hub and/or interventional device). For example, while the control mechanism is in a particular drive mode (e.g., the first drive mode or second drive mode), a user can temporarily link the first joystick 5332 to a corresponding hub and/or interventional device, such as a guide catheter (e.g., guide catheter 31 or guide catheter 2906), by pressing and holding one of the plurality of buttons (e.g., one of the shoulder or bumper buttons 5352, 5353, 5354, 5355). For example, in some embodiments, a user can temporarily link the first joystick 5332 to a guide catheter by pressing and holding the button 5352. In certain embodiments, while the bumper button 5352 is pressed, translation of the first joystick 5332 can cause guide catheter to translate accordingly. When the first bumper button 5352 is released, the first joystick 5332 can be automatically linked again to the corresponding hub and/or interventional device according to the particular drive mode.

In certain embodiments, if an interventional device is linked the first joystick 5332 in a particular drive mode and a user input is made to temporarily link the interventional device to the second joystick 5334, the interventional device can be temporarily unlinked from the first joystick 5332 while temporarily linked to the second joystick 5334. Similarly, in certain embodiments, if an interventional device is linked the second joystick 5334 in a particular drive mode and a user input is made to temporarily link the interventional device to the first joystick 5332, the interventional device can be temporarily unlinked from the second joystick 5334 while temporarily linked to the first joystick 5332.

As another example, while the control mechanism is in a particular drive mode or operated without a particular drive mode, a user can temporarily link the second joystick 5334 to a corresponding hub and/or interventional device, such as a procedure catheter (e.g., catheter 29, catheter 120, or catheter 2904) or an access catheter (e.g., catheter 124 or catheter 2902), by pressing and holding one of the buttons of the control mechanism (e.g., one of the plurality of shoulder or bumper buttons 5353, 5354, 5355). For example, the user can temporarily link the second joystick 5334 to a procedure catheter by pressing and holding the bumper button 5353 and link the second joystick 5334 with the access catheter by pressing and holding bumper button 5355. While the bumper button 5353 or bumper button 5355 is pressed, translation of the second joystick 5334 can cause the procedure catheter or the access catheter, respectively, to translate accordingly. When the bumper button 5353 or the bumper button 5355 are released, the second joystick 5334 can be automatically linked again to the corresponding hubs and/or interventional devices according to the particular drive mode.

In some embodiments, a user can select to link one of the first joystick 5332 and the second joystick 5334 with a plurality of hubs and/or interventional devices that differ from the default hubs and/or interventional devices for a current drive mode. For example, while the control mechanism is in a particular drive mode, a user can temporarily link the second joystick 5334 to a combination of different hubs and/or interventional devices, such as a procedure catheter (e.g., catheter 29, catheter 120, or catheter 2904) and an access catheter (e.g., catheter 124 or catheter 2902) by pressing and holding both the bumper button 5353 and the bumper button 5355 at the same time. While the bumper button 5353 and bumper button 5355 are pressed, translation of the second joystick 5334 can cause the procedure catheter and the access catheter to translate accordingly. When the bumper button 5353 and the bumper button 5355 are released, the second joystick 5334 can be automatically linked again to the corresponding hubs and/or interventional devices according to the first and/or second drive modes.

While the above examples describe linkage of particular hubs and/or interventional devices to a joystick by pressing and holding buttons, such as bumper buttons, in other embodiments, a particular hub and/or interventional device may be linked with a particular joystick after pressing and releasing a button, for example, until the button is pressed again or another button is pressed.

While the above examples, describe particular drive modes, in other embodiments, there may not be separate selectable drive modes. Instead, users may select which hubs and/or interventional devices are assigned to each of the joystick 5332 and joystick 5334 as described herein, for example, by pressing and/or holding one or more buttons on the control mechanism 5300. In some embodiments, the joystick 5332 and the joystick 5334 may each have default hubs and/or interventional devices or combinations of hubs and/or interventional devices linked thereto.

In operation, the corresponding coupled hubs and/or interventional devices linked to the first joystick 5332 or the second joystick 5334 can move axially and/or rotationally in response to actuation (e.g., movement) of the first joystick 5332 or the second joystick 5334.

Using the first joystick 5332 as an example, if the user moves (e.g., rolls) the first joystick 5332 in a direction along an axis A5, one or more hubs and/or interventional devices linked to the first joystick 5332 may move responsively in a corresponding axial direction, for example, at a predefined linear velocity. A user can advance and/or retract the linked hubs and/or interventional devices by moving the first joystick along the axis A5. In certain embodiments, movement of the joystick 5332 along the axis A5 may be referred to as forward and backward movement. For example, movement along the axis A5 in the direction of the button 5352 may be referred to as forward movement, and movement along the axis A5 in the direction opposite of the button 5352 may be referred to as backward movement. In some embodiments, forward movement of the joystick 5332 can correspond to distal movement or insertion of a linked interventional device. Backward movement of the joystick 5332 can be referred to as proximal movement or retraction of a linked interventional device.

In some embodiments, the predefined linear velocity can vary according to the extent of movement of the joystick 5332. For example, further movement of the joystick 5332 along the axis A5 from a starting position or neutral position can result in greater linear velocities. In other embodiments, the linear velocity may not vary as a result of the extent of movement of the joystick 5332.

In some embodiments, the linear velocity of one or more interventional devices linked with the joystick 5332 can be mapped linearly to the joystick (e.g., so that the linear velocity corresponds linearly to the throw of the joystick 5332. In other embodiments, the linear velocity of one or more of the interventional devices linked with the joystick 5332 can be mapped to the joystick 5332 using a non-linear cubic function, a step function, a combination of step and linear mapping, or other non-linear functions.

In some embodiments, one of the plurality of buttons of the control mechanism 5300, for example, button 5342, can be actuated to change (e.g., increase, decrease) the predefined linear velocity of the linked hubs and/or interventional devices. For example, pressing the button 5342 can toggle between two or more predefined linear velocities. In some embodiments in which the predefined linear velocity can vary according to the extent of movement of the joystick 5332, it may be possible for a user to change a range of predefined linear velocities over which movement of the joystick 5332 can cause the linked hubs and/or interventional devices to translate (e.g., by pressing a button). For example, a first range of linear velocities may be between 0-10 mm/second and a second range may be between 10-20 mm/second. A button, such as button 5342 may be pressed to change the range of linear velocities from the first range to the second range. In some embodiments, the same or a different button may be selected to change from the second range to the first range. In some embodiments, a third range of linear velocities (e.g., between 20-30 mm/second) may also be selectable (e.g., by pressing the button 5342 when the control mechanism is already operating within the second range of linear velocities). The control mechanism may be configured to operate using any suitable number of ranges of linear velocities. In some embodiments, the predefined linear velocities and/or ranges of linear velocities may change in response to a change in drive mode.

In some embodiments, the linked hubs and/or interventional devices may continue to move in the same direction at the predefined linear velocity until the user releases (e.g., stops manipulating) the first joystick 5332 or further moves the joystick along the axis A5.

In some embodiments, if the user moves (e.g., rolls) the first joystick 5332 in a direction along axis A4, one or more hubs and/or interventional devices linked to the first joystick 5332 can move responsively. For example, one or more interventional devices linked to the first joystick can move in a corresponding rotational direction, for example, at a predefined rotational velocity. In some embodiments, movement of the first joystick 5332 along axis A4 can cause a corresponding movement of a torque element of a hub (e.g., a gear or gear train) that can cause rotational movement of any interventional device coupled thereto. A user can rotate linked interventional devices by moving the first joystick 5332 along axis A4. In some embodiments, movement of the joystick along the axis A4 may be referred to as sideways or left/right movement. For example, movement along the axis A4 in the direction towards a central axis 5380 can be referred to as rightward movement and movement of the joystick along the axis A4 in the direction away from the central axis 5380 can be referred to as leftward movement. With respect to the second joystick 5334, movement along the axis A4 away from the central axis 5380 may be referred to as rightward movement, and movement along the axis A4 towards the central axis 5380 may be referred to as leftward movement. In certain embodiments rightward movement of the joystick may correspond to clockwise rotation and leftward movement may correspond to counterclockwise rotation.

In some embodiments, the predefined rotational velocity can vary according to the extent of movement of the joystick 5332. For example, further movement of the joystick 5332 along the axis A4 from a starting position can result in greater rotational velocities. In other embodiments, the rotational velocity may not vary as a result of the extent of movement of the joystick 5332.

In some embodiments, one of the plurality of buttons (e.g., 5342, 5343, 5344, 5345, 5346, 5357) can change (e.g., increase, decrease) the predefined rotational velocity of the linked hubs and/or interventional devices. In some embodiments, the same button that changes the linear velocity may change the rotational velocity. In other embodiments, a different button may change the rotational velocity. For example, in some embodiments, pressing the button 5342 can toggle between two or more predefined rotational velocities. In some embodiments in which the predefined rotational velocity can vary according to the extent of movement of the joystick 5332, it may be possible for a user to change a range of predefined rotational velocities over which movement of the joystick 5332 can cause the linked interventional devices to rotate (e.g., by pressing a button). For example, a first range of rotational velocities may be between 0-5 degrees/second and a second range may be between 5-10 degrees/second. A button, such as button 5342 may be pressed to change the range of rotational velocities from the first range to the second range. In some embodiments, the same or a different button may be selected to change from the second range to the first range. In some embodiments, a third range of rotational velocities (e.g., between 10-15 degrees/second) may also be selectable (e.g., using the button 5342). The control mechanism may be configured to operate using any suitable number of ranges of rotational velocities. In some embodiments, the predefined rotational velocities and/or ranges of rotational velocities may change in response to a change in drive mode.

In some embodiments, the linked interventional devices may continue to move in the same direction at the predefined rotational velocity until the user releases (e.g., stops manipulating) the first joystick 5332 or further moves the joystick along the axis A4.

In some embodiments, only certain interventional devices may be configured to rotate. For example, in some embodiments, a guidewire and an access catheter may be configured to rotate, but a guide catheter and a procedure catheter may not rotate. In such embodiments, if an interventional device that is configured to rotate and an interventional device that is not configured to rotate are both linked to a joystick, movement of the joystick along the axis A4 may cause rotation of only the interventional device that is configured to rotate.

While embodiments in which movement of the joystick 5332 along the axis A5 causes axial movement and movement of the joystick 5332 along the axis A4 causes rotational movement are described herein, in other embodiments, movement of the joystick 5332 along the axis A4 can cause axial movement and movement of the joystick 5332 along the axis A5 can cause rotational movement.

In certain embodiments, the joystick 5332 can be shaped, textured, dimensioned, or otherwise configured to prevent undesired movement along one of the axis A4 and the axis A5 during movement along the other of the axis A4 and the axis A5 (e.g., to prevent unintended rotation when translation is desired or vice versa). For example, as shown the joystick 5332 can have an elongate protruding band 5336 extending parallel and colinear with the axis A5 and perpendicular with the axis A4. The joystick 5332 may have an additional protrusion 5337 protruding from the band 5336. The protrusion 5337 may extend parallel and colinear with axis A4 when the joystick is in a starting position, as shown in FIG. 8A. In certain embodiments, when the joystick 5332 is in the starting position, a center of the protrusion 5337 may be positioned at the intersection of the axis A4 and the axis A5. In certain embodiments, in use, a user may push the protrusion 5337 to move the joystick 5332 along the axis A5 to translate one or more interventional devices. A user may push a side surface of the protrusion 5337 or of the protruding band 5336 to move the joystick 5332 along the axis A4 to rotate one or more interventional devices. The shape of the joystick 5332 may reduce the likelihood that a user will inadvertently move the joystick 5332 along an axis offset from either the axis A4 or the axis A5.

In some embodiments, simultaneous axial movement and rotational movement of an interventional device may be desirable. The joystick 5332 may be configured to move in a direction having directional components along both the axis A4 and the axis A5 (e.g., along an axis offset from both the axis A4 and the axis A5) to allow for both axial movement and rotational movement simultaneously. While a description of the first joystick 5332 has been provided herein, one of skill in the art would understand that the second joystick 5334 can have any of the same or similar features and/or functions. Additionally, in some embodiments, the first joystick 5332 and/or second joystick 5334 may be other types of controls (e.g., dials, buttons, scroll wheels, touch pads, etc.).

In some embodiments, the control mechanism 5300 may be provided with one or more fluidics controls for controlling components of a fluidics system, for example, to initiate and/or terminate the introduction of fluids to a catheter (e.g., saline, contrast, etc.). For example, the controller can include one or more contrast controls (e.g., button 5345, button 5347) that when actuated cause contrast media to be injected through one of the interventional devices linked to the control mechanism. In some embodiments, the buttons 5345 and 5347 may be associated with particular interventional devices and/or particular hubs so that actuation will cause contrast injection through any interventional device coupled to those hubs. In some embodiments, contrast injection may be linked to the hubs and/or interventional devices associated with a particular joystick such that actuation of a contrast injection button (e.g., button 5345 and 5347) can cause contrast injection via the hubs and/or interventional devices linked with the particular joystick. In some embodiments, a selection of a particular interventional device may be performed prior to actuation of the contrast controls. For example, a user may press and/or hold a button associated with a particular interventional device (e.g., button 5355 associated with an access catheter) and then actuate the contrast control (e.g., button 5345 or 5347) to cause contrast injection through the particular interventional device. In certain embodiments, the system may be configured to supply contrast through an inner most catheter upon actuation of a contrast control.

In some embodiments, the control mechanism 5300 can include one or more aspiration controls (e.g., button 5346). For example, pressing the button 5346 can initiate aspiration, and pressing the button 5346 again may end aspiration through one or more of the hubs and/or interventional devices. In some embodiments, the button 5346 may be associated with a particular hub and/or interventional device, such as a procedure catheter hub and/or procedure catheter. In some embodiments, aspiration may be linked to the hubs and/or interventional devices associated with a particular joystick such that actuation of an aspiration control (e.g., button 5346) can cause contrast injection via the hubs and/or interventional devices linked with the particular joystick. In some embodiments, a selection of a particular interventional device may be performed prior to actuation of the aspiration control. For example, a user may press and/or hold a button associated with a particular interventional device (e.g., button 5353 associated with a procedure catheter) and then actuate the aspiration control (e.g., button 5353) to cause aspiration through the particular interventional device. In certain embodiments, the system may be configured to supply aspiration through an inner most catheter upon actuation of an aspiration control.

In an example procedure using the control mechanism 5300, a user may begin with the control mechanism 5300 in the first drive mode or may select the first drive mode, for example, by pressing button 5344. In the present example, in the first drive mode, the joystick 5332 is linked with a guidewire and the joystick 5334 is linked with an access catheter, a procedure catheter, and a guide catheter.

The user may control the first joystick 5332 to advance the guidewire from a femoral access point into the aortic arch and adjacent a desired ostium. Next, the user may control the second joystick 5334 to advance the access catheter, procedure catheter, and guide catheter from the femoral access point into the aortic arch and adjacent the desired ostium.

After the guidewire, access catheter, procedure catheter, and guide catheter are positioned adjacent the desired ostium, the user may control the first joystick 5332 to rotate the guidewire so that the guidewire enters the desired ostium.

After the guidewire is positioned within the desired ostium, the use can control the second joystick 5334 to advance the access catheter, procedure catheter, and guide catheter into the desired ostium.

In some embodiments, the access catheter may be driven separately out of procedure catheter and guide catheter to engage the ostium first. For example, the user may press and/or hold the shoulder button 5355 so that the second joystick 5334 controls movement of the access catheter. In some embodiments, the user may control the second joystick 5334 to rotate the access catheter to enter the desired ostium. In some embodiments, after the access catheter is positioned within the desired ostium, the user may control the first joystick 5332 to advance the guidewire distally into the ostium to secure access. After the access catheter and guidewire are positioned within the desired ostium, the user can control the procedure catheter and/or the guide catheter to advance the procedure catheter and/or guide catheter into the ostium (and, in some embodiments, beyond), while using the support of the access catheter and/or guidewire to maneuver through the aorta and into the ostium. For example, in some embodiments, the user may press and/or hold the shoulder button 5352 to link the first joystick 5332 with the guide catheter and/or press and/or hold the shoulder button 5353 to link the second joystick 5334 with the procedure catheter. The user may then advance the guide catheter and/or the procedure catheter using the first and/or second joysticks.

In some embodiments, in the first drive mode, a user may press and/or hold the shoulder button 5352 so that the first joystick controls only the guide catheter. In certain embodiments, when the first joystick controls only the guide catheter when the control mechanism is in the first drive mode, the second joystick 5334 can control the procedure catheter and the access catheter. In some embodiments, the user may choose to advance the procedure catheter and access catheter together into the desired ostium prior to the guide catheter.

In certain embodiments, after supra-aortic access is achieved, the control mechanism can be switched to the second drive mode, for example, by a user pressing button 5344. In the present example, in the second drive mode, the first joystick 5332 is linked with the guidewire and the second joystick 5334 is linked with the procedure catheter and the guide catheter.

In certain embodiments, the user can control the first joystick 5332 to advance the guidewire to a procedure site. The user can control the second joystick 5334 to advance the procedure catheter and guide catheter to the procedure site while the access catheter remains by the ostium for support. In some embodiments, the guidewire and procedure catheter and guide catheter can be advanced to the procedure site in stages. For example, the guidewire may be advanced up to the petrous segment. The procedure catheter and guide catheter may then be advanced up to the petrous segment. The guidewire may then be advanced to the procedure site, and then the procedure catheter and guide catheter may be advanced up to the procedure site.

In some embodiments, it may be desirable to advance the guide catheter and procedure catheter separately to the procedure site. Additionally, in some embodiments, it may be desirable to advance only the procedure catheter all the way to the procedure site, while the guide catheter may be advanced only partially to the procedure site. In certain embodiments, the user may press and/or hold the shoulder button 5352 so that the first joystick 5332 controls only the guide catheter. In certain embodiments, when the first joystick 5332 controls only the guide catheter when the control mechanism is in the second drive mode, the second joystick 5334 can control only the procedure catheter.

In certain embodiments, if additional support is desired while advancing the procedure catheter and/or guide catheter to the procedure site, the access catheter can be advanced further distally towards the procedure site. For example, the user can press and/or hold the button 5355 to link the access catheter with the second joystick 5334, and can control the second joystick 5334 to advance the access catheter.

After the guide catheter and/or procedure catheter are positioned at the procedure site, the guidewire and the access catheter may be removed. For example, the user can control the first joystick 5332 to withdraw the guidewire. The user may press and/or hold the button 5355 to link the access catheter with the second joystick 5334 and control the second joystick 5334 to withdraw the access catheter.

After the guidewire and access catheter are withdrawn, the user can actuate button 5353 to apply aspiration through the procedure catheter, for example, to remove a clot at the procedure site. In certain embodiments, actuating the button 5353 may additionally apply aspiration through the guide catheter. In other embodiments, a second button may be actuatable to apply aspiration through the guide catheter. In some embodiments, a second aspiration control may be provided on a user interface instead of on the control mechanism 5300.

In some embodiments, it may be desirable to remove the procedure catheter from the treatment site prior to removing the guide catheter, for example, to perform additional aspiration using only the guide catheter. In those embodiments, the user may control only the procedure catheter, for example, by pressing and/or holding the button 5353 to link the procedure catheter with the second joystick 5334 so that movement of the second joystick controls movement of the procedure catheter. Alternatively, while in the second drive mode, the user may press and/or hold the shoulder button 5352 so that the first joystick 5332 controls only the guide catheter. As described above, in the second drive mode, when the first joystick 5332 controls only the guide catheter, the second joystick 5334 can control only the procedure catheter.

After aspiration is complete, the user may remove both the guide catheter and the procedure catheter (if the procedure catheter has not previously been removed), either together by controlling the second joystick 5334 when both the procedure catheter and guide catheter are linked thereto, or separately, for example, by pressing and holding the shoulder button 5352 so that the first joystick controls only the guide catheter and the second joystick controls only the procedure catheter.

In some embodiments, as the interventional devices are driven through the anatomy, tension can build between adjacent interventional devices. In some embodiments, it may be desirable to release the tension by controlling the adjacent interventional devices to drive in opposite directions. For example, a user may link a first of the adjacent interventional devices with the first joystick 5332 and a second of the adjacent interventional devices with the second joystick 5334 (if neither are already linked by themselves with their respective joysticks), and can operate the first joystick 5332 and the second joystick 5334 to move the first interventional device and the second interventional device in opposite directions (e.g., moving one proximally and one distally).

As described above, multiple hubs and/or interventional devices can be linked to a single control, such as the joystick 5332 and the joystick 5334. In certain embodiments, when multiple hubs and/or interventional devices are linked to a control, operation of the control to cause axial movement of the multiple hubs and/or interventional devices can cause the multiple hubs and/or interventional devices to move in in parallel. In other embodiments, the multiple hubs and/or interventional devices may move in series. For example, a first linked interventional device may move axially over a particular distance corresponding to the operation of the control, and then a second linked interventional device can move over the same distance. In other embodiments, the second linked interventional device may move over a scaled distance.

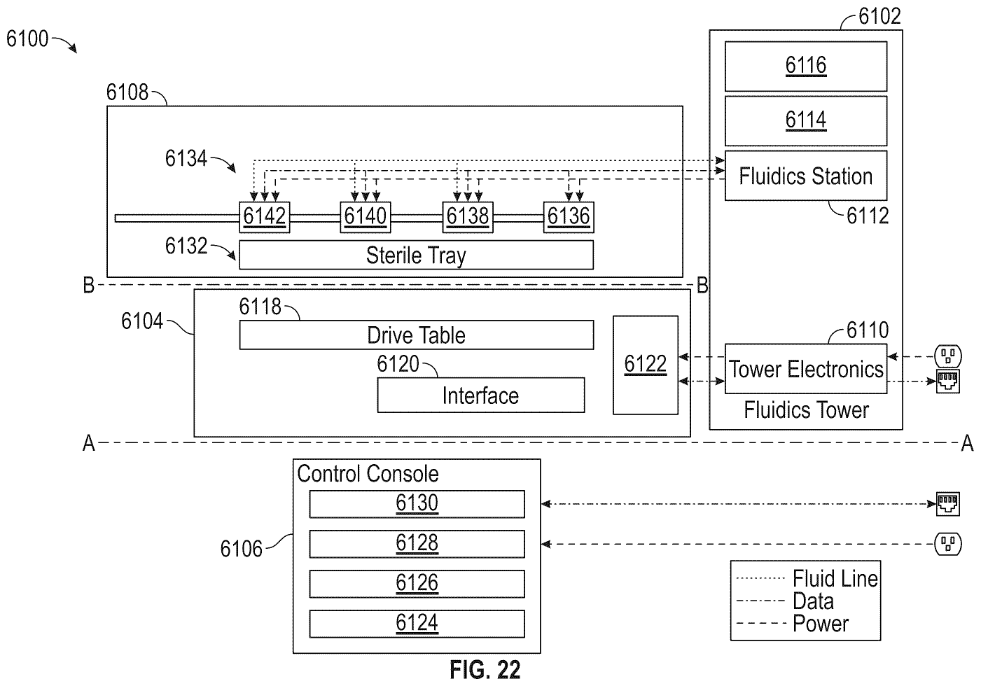

FIG. 22 illustrates a system diagram of an example of a medical device operation system 6100. The system diagram shows fluid and electrical connectivity between the subsystems of the medical device operation system 6100. The medical device operation system 6100 may include a fluidics tower 6102, a robotic drive system 6104, a control system 6106, and one or more interventional devices 6108.

The fluidics tower 6102 may be a housing or console comprising a fluidics management system for controlling the administration or removal of contrast, saline and/or bodily fluids to and/or from an interventional device. The fluidics tower 6102 may further include an electronics tower 6110, a fluidics station (or “system”) 6112, a monitor 6114, and one or more communication devices 6116. Although illustrated in FIG. 22 as part of the fluidics tower 6102, in other embodiments the electronics tower 6110 may be housed separately from the fluidics tower 6102 while still being in communication with the fluidics tower 6102, the interventional devices 6108, the robotic drive system 6104, and the control console system.

The electronics tower 6110 may be a housing configured to contain system electronics such as one or more processors and memory. The one or more processors and memory may be organized into one or more computer devices. The electronics tower 6110 may include a power cord configured to be operatively coupled to a power source, such as a battery, a generator, or an outlet. In some embodiments, the electronics tower 6110 may draw power from a source providing 110/220 volts of alternating current (VAC) power.

The system electronics may be a central hub for the medical device operation system 6100 interconnecting the electronic devices from other components as described in greater detail below. The system electronics of the electronics tower 6110 may be configured to transmit and receive electronic signals and/or data to operate components of the medical device operation system 6100. The electronics tower 6110 may include means for connecting to other devices. The electronics tower 6110 may transmit and/or receive electronic signals and/or data wirelessly or over a wired connection. In some embodiments, the electronics tower 6110 may include an ethernet port for connecting the system electronics to a network. In some embodiments, the electronics tower 6110 may include one or more ports for tethering to nearby electronic devices via a wired connection. For example, the electronics tower 6110 may have ports to run cables between the fluidics tower 6102 and the robotic drive system 6104 and/or control system 6106. Alternatively, the electronics tower 6110 may be configured to connect wirelessly to nearby electronic devices. For example, the electronics tower 6110 may include a personal area network (PAN) module, such as Bluetooth®, or other network capabilities to transmit data wirelessly.

In some embodiments, electronic signals and/or data may include instructions for a system, subsystem, component, or device to perform a particular task. Additionally and/or alternatively, electronic signals and/or data may include indicators or data measured from sensors for processing by a computing device.

In some embodiments, the electronics tower 6110 may connect to other devices over a communication network (“network”). The network may cover a small geographic area such as a particular room or building, a medium geographic area such as a city, or a large geographic area so long as there is access to a network. For example, the network may be a local area network (LAN) or wireless local area network (WLAN) comprising a series of devices linked together to form a network within a hospital or clinic. Alternatively, the network may be a wide-area network (WAN) comprising a series of devices linked together to form a network within a medical campus comprising two or more buildings. Alternatively, the network may be an intranet or internet for providing global connectivity. Connecting over the network advantageously connects the operating room to physicians located around the world, including experts located across the nation or in other countries, without requiring the physician to travel to the operating room. This advantageously connects patients to physicians without the time or cost required for the physician to physically travel to the operating room. In some procedures, every minute of delay before a procedure is performed can increase the chance of a bad outcome, and thus such surgical systems can help mitigate damage to the patient due to a delay in starting the surgical procedure.

The fluidics system 6112 may include one or more subsystems comprising one or more containers, one or more tubes, and one or more pumps. The subsystems may be divided and organized into a contrast subsystem, a saline subsystem, and/or an aspiration (or “vacuum”) subsystem. The fluidics system 6112 may be the fluidics system described above.

A contrast subsystem may be configured for supplying contrast to a patient. The contrast subsystem may include one or more containers for storing and supplying contrast, one or more fluid communication channels (“tubes”), one or more valves, and a high-pressure pump.

A saline subsystem may be configured for supplying saline to a patient. The saline subsystem may similarly include one or more containers for storing and supplying saline, one or more tubes, one or more valves, and one or more pumps.

An aspiration subsystem may be configured for removing biological material from a patient. The aspiration subsystem may include one or more containers, one or more tubes, one or more valves, and a vacuum pump.

The one or more pumps and containers of the contrast, saline, and aspiration subsystems may be contained with the fluidics tower 6102. The one or more tubes of the contrast, saline, and aspiration subsystems may extend out of the fluidics tower 6102 for interacting with and coupling to other devices.

The monitor 6114 may be any electronic visual computer display (or displays) that includes a screen and circuitry configured to interpret electronic signals to display one or more images. For example, the monitor 6114 may include an imaging window, a speed indicator, a rotational indicator, an axial position bar, a telescopic position window, one or more axial position indicators, and/or other graphical user interfaces or windows. In some embodiments, the monitor 6114 may be configured to display fluoroscopic images, catheter data, fluidics information (e.g., information relating to a contrast injection subsystem including its current operation status, information relating to a saline subsystem including its current operation status, and/or information relating to a aspiration subsystem including its current operation status) including current state information) providing saline, providing vacuum for aspiration), and patient data including vital signs. In some embodiments, the monitor 6114 may be the display 23 described above.

The one or more communication devices 6116 may be one or more microphones, one or more cameras, and/or one or more audio output devices such as a speaker and/or a headset.

The fluidics system 6112, the monitor 6114, and the one or more communication devices 6116 may be electrical communication with the electronics tower 6110 and configured to receive and/or transmit electronic signals and/or data therebetween. In some embodiments, the electronic signals and/or data may include instructions to activate one or more pumps and/or one or more valves of the fluidics system 6112. For example, the instructions may direct the fluidics system 6112 to provide saline and/or contrast to the one or more interventional devices 6108. In some embodiments, the data may include video and/or audio inputs and audio outputs for the monitor 6114 and one or more communication devices 6116. For example, the data may be one or more images to be displayed on the monitor 6114 and/or audio-visual data captured by the one or more communication devices 6116.

The fluidics tower 6102 may be further configured to be operatively coupled with the one or more interventional devices 6108. In some embodiments, the fluidics system 6112 may be mechanically coupled to and/or in fluid communication with the one or more interventional hubs 6134. Accordingly, activating the fluidics system 6112 may provide contrast, saline, and/or suction to the interventional hubs 6134 and corresponding interventional devices.

The robotic drive system 6104 may include a plurality of components to drive one or more access systems such as catheters and guidewires during a procedure. The robotic drive system 6104 may be the drive system 18 described above. The robotic drive system 6104 may include a drive table 6118, an interface 6120, and a joint setup 6122.

The drive table 6118 may support the one or more disposable devices 6108 (e.g., a catheter) configured to be advanced to access a patient for performing a surgical procedure and/or for introducing saline, contrast media or therapeutic agents, or providing aspiration. The drive table 6118 may further support a sterile barrier.

The drive table 6118 may be the support table 20 described above. The drive table 6118 may be positioned over or alongside a patient, and configured to axially advance, retract, and in some cases rotate two or three or more different concentrically oriented intravascular devices of an interventional device assembly. The drive table 6118 may include electronics and motors for controlling the location of the interventional devices and actuation of fluidics components.

In some embodiments, the drive table 6118 may include one or more hub adapters. The one or more hub adapters may include the drive magnets described above. Movement of the drive magnets may be driven by a drive system carried by the drive table 6118. Movement of the drive magnets may be configured to drive one or more interventional hubs 6134 of the one or more disposable devices 6108. The drive table 6118 and the one or more disposable devices 6108 may be separated such that the one or more interventional hubs 6134 may not mechanically couple to the drive table 6118 as shown by axis B-B.

The interface 6120 may be any device configured to interact with and/or display information to personnel locally situated within an operating room during a procedure, such as a bedside user. For example, a bedside user may be nurse or surgical technician staffed within the operating room. The interface 6120 may include an imaging window, a speed indicator, a rotational indicator, an axial position bar, a telescopic position window, and/or one or more axial position indicators. The interface 6120 may be the display 23 described above configured to display fluoroscopic images, catheter data, pressure values of the fluidics system, and/or other patient data. In some embodiments, the interface 6120 may be a touchscreen device such as a tablet computer. The interface 6120 may display information to a bedside user. The information displayed to the bedside user may include directions and/or prompts for the bedside user to follow. For example, the information may describe what steps to perform next, how to position the robotic drive system 6104, when to deploy the drapes, whether the system is malfunctioning or whether an error is detected, and/or prompt the bedside user to otherwise interact with the system. In some embodiments, the interface 6120 is configured to accept user input to control one or more components, for example, position of the drive table.

The interface 6120 may be in communication with one or more portions of the medical device operation system 6100 (for example, the robotic drive system 6104, the fluidics tower 6102, the disposable devices 6108, etc.). In some embodiments, the interface 6120 may be mechanically coupled to the robotic drive system 6104, be housed separately, or be mechanically coupled to another part of the medical device operation system 6100. The interface 6120 may control the joint setup 6122 of the robotic drive system 6104. For example, the interface 6120 may control the transition processes between a storage position and a deployed position, engaging a priming sequence, or controlling fine motor adjustments for providing minor adjustments to the positions of the interventional hubs. Controlling the joint setup 6122 and motors with the interface 6120 advantageously provides greater precision and setup before an operation by individuals present in the operating room.

The interface 6120 may advantageously provide a backup control mechanism to interact with and provide input to control the medical device operation system 6100, for example, in the event that the control system 6106 is rendered incapable of performing an operation.

The joint setup 6122 may include a plurality of joints and motors for controlling the positioning and movements of the robotic drive system 6104. In some embodiments, the joint setup 6122 may initialize the robotic drive system 6104 into a starting position. The initialization process may include transitioning the robotic drive system 6104 from a storage position to a deployed position and vice versa. For example, the joint setup 6122 may be configured to transition the robotic drive system 6104 from a storage position the robotic drive system 6104 to a deployed position such that at least a portion of the robotic drive system 6104 transitions from a compact state to a position where at least a portion of the robotic drive system 6104 is positioned either over or alongside a patient.

Within the robotic drive system 6104, the drive table 6118 may be mechanically coupled with the interface 6120 and the joint setup 6122. The joint setup 6122 may also be electrically connected to the drive table 6118 and the interface 6120. The robotic drive system 6104 may be configured for the joint setup 6122 to transmit electronic signals and data to the drive table 6118 and the interface 6120. Additionally and/or alternatively, the robotic drive system 6104 may be configured for the joint setup 6122 to receive electronic signals and data from the drive table 6118 and the interface 6120.

The control system 6106 may be a collection of components configured to control and operate the robotic control system described above. In some embodiments, the control system is a control console or is coupled to a control console. The control system 6106 may further include an operator controller 6124, an interface 6126, a monitor 6128, and one or more communication devices 6130. The control system 6106 may be locally positioned or remotely positioned. For example, in some embodiments, the control system 6106 may be located in the operating room with the fluidics tower 6102, the robotic drive system 6104, and the one or more disposable devices 6108. Alternatively, the control system 6106 may be located remotely (e.g., in a control room) as illustrated by line A-A. The control system 6106 may include system electronics comprising one or more processors and one or more memory components (“memory”). The system electronics may be configured to electrically connect the controller 6124, the interface 6126, the monitor 6128, and the one or more communication devices 6130.

The control system 6106 may include means for connecting to other devices. The control system 6106 may transmit and/or receive electronic signals and/or data wirelessly or over a wired connection. In some embodiments, the control system 6106 may include an ethernet port for connecting the system electronics to a network. In some embodiments, the control system 6106 may include one or more ports for tethering to nearby electronic devices via a wired connection. For example, the control system 6106 may have ports to run cables between the control system 6106 and the fluidics tower 6102 and/or robotic drive system 6104. Alternatively, the control system 6106 may be configured to connect wirelessly to nearby electronic devices. For example, the control system 6106 may include a Bluetooth® module or other network capabilities to transmit data wirelessly.

In some embodiments, the control system 6106 may connect to other devices over a network as described above.

The controller 6124 may be any device configured to enable a surgeon to control portions of the medical device operation system 6100 in the same location as the patient. For example, the controller 6124 may be any of the control mechanisms or controllers described herein (e.g., controller 5310). The controller 6124 may enable a user to control portions of the fluidics tower 6102, the interventional devices 6108, and the robotic drive system 6104. For example, the controller 6124 may be configured to move the to desired positions to perform a procedure on a patient as described herein.

The controller 6124 may be part of the control system 6106 or connected, wirelessly or via a wired connection, to the control system 6106.

The interface 6126 may be configured to display information to the surgeon. The interface 6126 may be the display 23 described above configured to display fluoroscopic images, catheter data, or other patient data. The interface 6126 may be a touchscreen device. The interface 6126 be a graphical user interface as described, for example, with respect to FIGS. 9-21.

The monitor 6128 may include one or more electronic displays. The monitor 6128 may be any electronic visual computer display that includes a screen and circuitry configured to interpret electronic signals to display one or more images. The monitor may display the interface 6126. In some embodiments, the monitor 6128 may be configured to display fluoroscopic images, catheter data, or other patient data. Alternatively, the monitor 6128 may be configured to display one or more views of the operating room. For example, the monitor 6128 may be configured to display the working area during a procedure by displaying only the surgical site. In another example, the monitor 6128 may display the entire operating room including the surgical technicians. In another example, the monitor 6128 may display more than one view. Displaying a plurality of views to capture the entire operating room may advantageously enhance communication and understanding between the physician and the technicians and/or assistants located in the operating room thereby increasing the efficiency and safety of procedures.

The one or more communication devices 6130 may be any one or more microphones, one or more cameras, and/or one or more audio output devices.

As shown in FIG. 22, the fluidics tower 6102, the 6104, the control system 6106, and the one or more disposable devices 6108 are connected to a power source. In some embodiments, the fluidics tower 6102 and the control system 6106 may be directly connected to a power source such as an outlet. In some embodiments, the robotic drive system and the one or more disposable devices 6108 may indirectly connect to a power source. For example, the robotic drive system 6104 and the one or more disposable devices 6108 may receive power from the fluidics tower 6102. In such embodiments, the joint setup 6122 of the robotic drive system 6104 may be electrically connected to the electronics tower 6110 of the fluidics tower and the interventional hubs 6134 of the one or more disposable devices 6108 may be electrically connected to the fluidics system 6112 of the fluidics tower 6102 such that power may be transmitted therebetween.

Furthermore, as shown in FIG. 22, the fluidics tower 6102, the robotic drive system 6104, the control system 6106, and the one or more disposable devices 6108 may be electrically connected and configured to share electrical signals and/or data. In some embodiments, the fluidics tower 6102 may be electrically connected and configured to share electrical signals and/or data with the robotic drive system 6104, the control system 6106, and the one or more disposable devices 6108. For example, the electronics tower 6110 of the fluidics tower 6102 may be electrically connected with the joint setup 6122 of the robotic drive system 6104 and the control system 6106 while the fluidics system 6112 of the fluidics tower 6102 may be electrically connected with the one or more interventional hubs 6134 of the one or more disposable devices 6108. In some embodiments, the electronics tower 6110 may be electrically connected with the control system 6106 via a network, as shown in FIG. 22.

The one or more interventional hubs 6134 may include a first interventional hub 6136, a second interventional hub 6138, a third interventional hub 6140, and a fourth interventional hub 6142. In some embodiments, the one or more interventional hubs 6134 may be aligned sequentially such that the first interventional hub 6136 may be positioned at a first end and the fourth interventional hub 6142 may be positioned at a second end opposite the first end. In some embodiments the first end may be a proximal end closest to a patient and the second end may be a distal end furthest from the patient. A sterile tray 6132 may separate the one or more interventional hubs 6134 and corresponding interventional devices from a support table. In some embodiments, the sterile tray 6132 forms a sterile barrier, such as the sterile barrier 32 described above.

In some embodiments, the first interventional hub 6136 may be a guidewire hub, such as the guidewire hub 26 described above; the second interventional hub 6138 may be a first catheter hub, such as the access catheter hub 2910 described above; the third interventional hub 6140 may be a second catheter hub configured to engage with and guide a procedure catheter, such as the procedure catheter hub 2912 described above; and the fourth interventional hub 6142 may be a third catheter hub configured to engage with and guide a guide catheter, such as the guide catheter hub 2914. In some embodiments, the guide catheter may extend distally from the fourth interventional hub 6142.

The fluidics tower 6102 may be electrically connected to the robotic drive system 6104, the control system 6106, and the one or more disposable devices 6108 wherein electrical signals and/or data may be transmitted between therebetween as discussed in greater detail below. The local system may transmit information about the fluidics system 6112, the robotic drive system 6104, and the plurality of interventional devices 6108 to the control system 6106 via the fluidics tower 6102.

The one or more communication devices 6130 may be in electrical communication with a power source. The one or more communication devices 6130 may further be in electrical communication with the electronics tower 6110 and configured to receive and/or transmit electronic signals and/or data therebetween. In some embodiments, the one or more communication devices 6130 is in electrical communication with the electronics tower 6110 via a network. For example, the one or more communication devices 6130 may be electrically coupled to an ethernet cable configured to connect the one or more communication devices 6130 to a network, wherein the electronics tower 6110 may be electrically coupled to the network.

FIGS. 9-21 illustrate embodiments of a user interface coupled to or part of a control system (e.g., control system 6106, control system 3120) for controlling one or more interventional devices. In some embodiments, the user interface can be coupled to or part of a control console. In some embodiments, information regarding user inputs from a controller (e.g., controller 5310) may be provided (e.g., from one or more sensors of a sensor system, such as sensors 3110a) to the control system and used (e.g., by one or more hardware processors, such as processor(s) 3120a) to generate a user interface. In some embodiments, information regarding the interventional devices, hubs, hub adapters, and/or drive table may be provided from one or more subsystems or components (e.g., fluidics tower 6102, robotic drive system 6104, interventional devices 6108, etc.) to the control system and/or control console and used (e.g., by one or more hardware processors) to generate a user interface. For example, in some embodiments, information regarding the interventional devices, hubs, hub adapters, and/or drive table may be provided from one or more sensors of a sensor system (e.g., sensors 3135 and sensors 3130a) to the control system and/or control console and used (e.g., by one or more hardware processors) to generate a user interface. As used herein, “sensor system” may refer collectively to the one or more sensors (e.g., sensors 3110a, sensors 3135, and/or sensors 3130a) that may be provide information regarding a medical procedure as described herein. The sensor system can include one or more linear position sensors, such as a linear potentiometer, a linear variable differential transformer (LVDT) sensor, an ultrasonic sensor, a laser sensor, and/or a hall effect sensor, to measure axial movement of each of the interventional devices 3130. In some cases, the sensor system can include a magnet, a magnet resistive element, an encoder, a potentiometer, a hall effect sensor, rotary variable differential transformer (RVDT) sensor, or a combination thereof to detect an angular position and/or measure a rotational movement of each of the interventional devices.

As shown in FIG. 9, the user interface 5400 can include a plurality of windows. For instance, the user interface 5400 can include an imaging window 5410, an instrument window 5420, a notification window 5430, a first video feed window 5440, a second video feed window 5450. In some instances of the user interfaces, only some of the components shown in FIG. 9 are displayed. In some cases, the user interface 5400 can be displayed on a capacitive screen, a resistive screen, a touch pad, or other touch-based sensing device. This can beneficially allow users to interact with the visual elements displayed on the user interface 5400. For example, in some embodiments, in addition to the user interface 5400 being displayed, users can adjust each of a plurality of parameters (e.g., speed, rotational direction, axial position) displayed on the user interface 5400 by interacting with the user interface 5400. As further described below, the plurality of parameters can be displayed on the instrument window 5420. Users can interact with the user interface 5400 by, for example, using basic gestures such as tapping, swiping, and/or dragging the components displayed on the capacitive screen, resistive screen, touch pad, or touch-based sensing device. Additionally or alternatively, the parameters displayed on the user interface 5400 can be adjusted by manipulating one or more physical buttons/components positioned adjacent the display in which the user interface 5400 is displayed. In some cases, at least one window of the plurality of windows of the user interface 5400 can display a patient's vital signs (e.g., body temperature, pulse rate, respiration rate, blood pressure, blood oxygen, etc.). In some embodiments, at least one window of the plurality of windows can include user profiles and/or settings. In some cases, instructions on how to move and/or operate the interventional devices associated with the user interface 5400 can be shown on the user interface 5400.

In some cases, the imaging window 5410 can be configured to display a live feed of the images and/or video generated by an imaging system 3142. The imaging window 5410 can include more than one live feed. For instance, the imaging window 5410, can include four live feeds of the images and/or video generated by the imaging system 3142. Each of the live feeds of the imaging window 5410 can include a live feed showing fluoroscopic imaging of different parts of the body patient and/or taken from different angles. Although reference is made to the imaging window 5410 having four live feeds, the imaging window 5410 can display more than or less than four live feeds. The first video feed window 5440 and the second video feed window 5450 can each provide live feed of, for example, the clinician performing the procedure, the location where the procedure is taking place, the medical devices used in the operating room, and/or the control mechanism used for the procedure. In some embodiments, more live feeds may be available than are displayed by the imaging window 5410. In such embodiments, the user interface may change the live feeds shown in the window 5410, for example, in response to a user selection. In certain embodiments, one or more of the live feeds may also be expanded over a larger portion of the window 5410, for example, in response to a user input. In some cases, in response to a user selecting a point on the imaging window 5410 corresponding to a location along a vasculature of a patient as represented by the fluoroscopic imaging, one or more of the interventional devices associated with the instrument window 5420 may be automatically driven to the location along the vasculature.

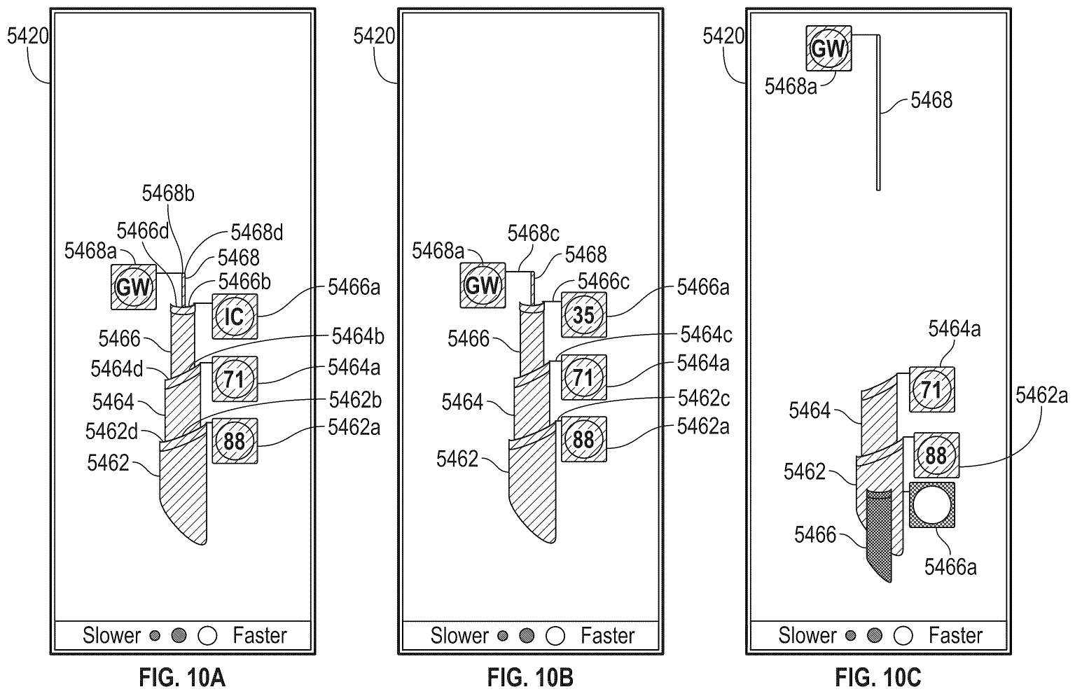

The instrument window 5420 can provide a visual indication of the status of one or more interventional devices. For example, the instrument window 5420 can display what interventional devices, if any, are active and/or associated to respective hubs of the drive system (e.g., drive system 18). This can beneficially allow clinicians to easily identify which interventional devices are available and the position of the interventional devices relative to each other. As shown in FIGS. 10A-10C, the instrument window 5420 can include a plurality of interventional device representations 5462, 5464, 5466 and 5468. In some embodiments, each of the plurality of interventional device representations can represent a particular interventional device coupled to a drive table in communication with the user interface (e.g., via a control system or control console). In certain embodiments, the control system or control console may receive information, related to the particular interventional devices (e.g., identify information, shape and/or size information, axial and/or rotational position of the interventional devices, information regarding an order of devices, axial and/or rotational velocity of the interventional devices, magnitudes and/or directions of axial and/or rotational movement, etc.) via a sensor system including one or more sensors (e.g., sensors 3135, sensors 3130a). The received information can be used (e.g., by one or more hardware processors) to generate a user interface providing information about the particular interventional devices. For example, the identify information can be used (e.g., by one or more hardware processors) to provide an indication of the particular interventional devices represented by the interventional device representations (e.g., by generating a user interface including the identify information).

In certain embodiments, each of the interventional device representations 5462, 5464, 5466 and 5468 may have visual characteristics that correspond to characteristics of the interventional devices to which they correspond. For example, each interventional device representation 5462, 5464, 5466 and 5468 may be shaped and/or sized (e.g., on a scaled or proportional basis) to correspond to the shape and/or size of the interventional device to which it corresponds or a portion of the interventional device to which it corresponds (e.g., a distal section). In some embodiments, the interventional device representations 5462, 5464, 5466 and 5468 may be displayed in relative positions corresponding to the relative positions of the interventional devices to which they correspond. For example, a distance between the distal ends of two interventional device representations may correspond (e.g., on a 1:1 or scaled basis) to a distance between the distal ends of the two interventional devices to which the interventional device representations correspond.

Each of the plurality of interventional device representations 5462, 5464, 5466 and 5468 can include a marker 5462a, 5464a, 5466a, 5468a. The markers 5462a, 5464a, 5466a, 5468a can provide an indication of the specific interventional devices each of the plurality of interventional device representations 5462, 5464, 5466 and 5468 are associated with. For example, each marker can include a character or set of characters identifying the interventional device its respective interventional device representation is associated with.

For example, as shown in FIGS. 10A-10C, the marker 5462a can provide an indication (e.g., the label “88”) that the interventional device representation 5462 is associated with a guide catheter (e.g., guide catheter 31), which may be a guide catheter having a 0.088 inch inner diameter. As another example, the marker 5464a can provide an indication (e.g., the label “71”) that the interventional device representation 5464 is associated with a procedure catheter (e.g., catheter 120), which may be a procedure catheter having a 0.071 inch inner diameter. In some embodiments, the marker 5466a can provide an indication (e.g., the label “IC” as shown in FIG. 10A) that the interventional device representation 5466 is associated with an insert catheter (e.g., insert or access catheter 2902). In some embodiments, the marker 5466a can provide an indication (e.g., the label “35” as shown in FIG. 10B) that the interventional device representation 5466 is associated with a second procedure catheter (e.g., catheter 124), which may have a 0.035 inch inner diameter. The marker 5468a can provide an indication (e.g., the label “GW”) that the interventional device representation 5468 is associated to a guidewire (e.g., guidewire 27 or guidewire 2907).

Each of the interventional device representations 5462, 5464, 5466 and 5468 can include a visual indication 5462b, 5464b, 5466b, and 5468b of a distal end of the interventional device representation, as shown in FIG. 10A. Each of the visual indications 5462b, 5464b, 5466b, and 5468b can include a shape corresponding to a distal shape of the interventional device associated with the interventional device representations 5462, 5464, 5466 and 5468. The shape may include a distal tip or edge 5462d, 5464d, 5466d, 5468d. The edge 5462d, 5464d, 5466d, 5468d may be a rounded (e.g., semi-circular) top edge, a flat edge, or a beveled edge. In certain embodiments, the shape of the visual indication 5462b, 5464b, 5466b, and 5468b can include a beveled surface, which can correspond to a shape of the distal end of the interventional device (e.g., a guide catheter) associated with the interventional device representation 5462. As another example, the shape of the visual indication 5466b can include a surface which can be substantially perpendicular with a longitudinal axis of the interventional device representation 5466.

In some embodiments, a distance between a distal tip or edge 5462d, 5464d, 5466d, 5468d of a first visual indication 5462b, 5464b, 5466b, and 5468b and a distal tip or edge 5462d, 5464d, 5466d, 5468d of a second visual indication 5462b, 5464b, 5466b, and 5468b can provide an indication about the distance between the distal end of the interventional device associated with the first visual indication and the interventional device associated with the second visual indication. In some embodiments, a distance between a point 5462e, 5464e, 5466e, and 5468e along the shape of a first visual indication 5462b, 5464b, 5466b, and 5468b and a point 5462e, 5464e, 5466e, and 5468e along the shape of a second visual indication 5462b, 5464b, 5466b, and 5468b can provide an indication about the distance between the distal end of the interventional device associated with the first visual indication and the interventional device associated with the second visual indication.

In some embodiments, at least some of the points 5462e, 5464e, 5466e, and 5468e are aligned along a longitudinal axis parallel with the axis 5480. In some embodiments, at least some of the points 5462e, 5464e, 5466e, and 5468e are positioned along the central longitudinal axis 5480. In some embodiments, at least some of the points 5462e, 5464e, 5466e, and 5468e are position along the distal tip or edge 5462d, 5464d, 5466d, and 5468d of the visual indications. In some embodiments, at least some of the points 5462e, 5464e, 5466e, and 5468e are positioned along a top most or distal most portion of the shapes of the visual indication 5462b, 5464b, 5466b, and 5468b. In some embodiments, at least some of the points 5462e, 5464e, 5466e, and 5468e are positioned at a distal most location along the distal tip or edge 5462d, 5464d, 5466d, and 5468d of the shape of the visual indication 5462b, 5464b, 5466b, and 5468b.

The distance between at least two of the visual indications 5462b, 5464b, 5466b, and 5468b can be measured from a point 5462e, 5464e, 5466e, and 5468e along the shape of one of the visual indications and a point 5462e, 5464e, 5466e, and 5468e along the shape of another one of the visual indications. For instance, the distance can be measured from between a point on a top-most or distal most portion of a shape of a first visual indication and a point on a top-most or distal most portion of a shape of a second visual indication (e.g., along a central axis, such as axis 5480). In some cases, the distance can be measured from points on a distal-most portion of the shapes which are not along the central axis. In some embodiments, the distance is an axial distance between the two points measured along the axis 5480.

The distance between a point along the shape of a first visual indication 5462b, 5464b, 5466b, and 5468b and a point along the shape of a second visual indication 5462b, 5464b, 5466b, and 5468b can represent a scaled and/or a non-scaled distance between the distal end of a first interventional device associated with a first interventional device representation 5462, 5464, 5466 and 5468 having the first visual indication 5462b, 5464b, 5466b, and 5468b and the distal end of a second interventional device associated with a second interventional device representation 5462, 5464, 5466 and 5468 having the second visual indication 5462b, 5464b, 5466b, and 5468b. For example, a non-scaled distance can provide an indication that the distance between the points of the first visual indication and the second visual indication is the actual distance between the distal ends of the interventional devices associated with the first visual indication and the second visual indication. A scaled distance can provide an indication that the distance between the points of the first visual indication and the second visual indication represents a proportional distance of the actual distance between the distal ends of the interventional devices associated with the first visual indication and the second visual indication. For example, using a 2:1 scale, a distance of 2 cm between the points can provide an indication that the actual distance between the distal ends of the interventional devices associated with the first visual indication and the second visual indication is 1 cm. Although reference is made to a 2:1 scale, other scales can be used (e.g., 1:2; 1:3; 1:4; 1:10; 2:1; 3:1; 4:1; 10:1; etc.).

In some embodiments, the distance between a point along the shape of the first visual indication 5462b, 5464b, 5466b, and 5468b and a point along the shape of a second visual indication 5462b, 5464b, 5466b, and 5468b can represent an estimated scaled and/or a non-scaled distance between the distal end of a first interventional device associated with a first interventional device representation 5462, 5464, 5466 and 5468 having the first visual indication 5462b, 5464b, 5466b, and 5468b and the distal end of a second interventional device associated with a second interventional device representation 5462, 5464, 5466 and 5468 having the second visual indication 5462b, 5464b, 5466b, and 5468b. In some embodiments, the positions of the distal end of the first interventional device and the distal end of the second interventional device may be estimated based on detected positions (e.g., by the sensor system) of the hubs and/or hub adapters corresponding to the first interventional device and the second interventional device.

In some embodiments the visual indications 5462b, 5464b, 5466b, and 5468b can provide a visual indication about the position of the distal end of the interventional devices associated with the interventional device representations 5462, 5464, 5466 and 5468 with respect to each other. This can beneficially allow clinicians to identify the position of the interventional devices associated with the interventional device representations 5462, 5464, 5466 and 5468 with respect to each other during the interventional procedure.

In some cases, the instrument window 5420 can include a plurality of pointers 5462c, 5464c, 5466c, 5468c, as shown in FIG. 10B. Each of the plurality of pointers 5462c, 5464c, 5466c, 5468c can be positioned between one of the plurality of interventional device indicator representations 5462, 5464, 5466 and 5468 and one of the plurality of markers 5462a, 5464a, 5466a, 5468a. For instance, each of the plurality of pointers 5462c, 5464c, 5466c, 5468c can include a line extending between an interventional device representation 5462, 5464, 5466 and 5468 and interventional device markers 5462a, 5464a, 5466a, 5468a. Using the pointer 5462c as an example, the pointer 5462c can extend between the interventional device representation 5462 and the interventional device marker 5462a. In some cases, one end of the pointer 5462c can be connected to the interventional device representation 5462 and the other end can be connected to the interventional device marker 5462a. This can beneficially allow clinicians to identify which interventional device marker 5462a the interventional device representation is associated with, and vice versa. As will be discussed in further detail below, this can beneficially allow clinicians to identify the operational status and/or identity of each of the plurality of interventional device representations 5462, 5464, 5466 and 5468.

The markers 5462a, 5464a, 5466a, 5468a can include any character(s) (e.g., letters, numbers, or combination thereof) for providing an indication of the interventional device the interventional device representations 5462, 5464, 5466 and 5468 are associated with. Alternatively, or additionally, each of the interventional device representations 5462, 5464, 5466 and 5468 and/or the markers 5462a, 5464a, 5466a, 5468a can include a color, which can provide an indication of the interventional device the interventional device representations 5462, 5464, 5466 and 5468 are associated with. In some cases, the markers 5462a, 5464a, 5466a, 5468a may be empty (e.g., not show a color, letters, numbers, etc.) to provide an indication that an interventional device is not associated with an interventional device representations 5462, 5464, 5466 and/or 5468 (e.g., an interventional device is not installed to a respective hub or hub adapter of the drive system). For example, as shown in FIG. 10C, the marker 5466a can display an empty box to indicate that the interventional device representation 5466 is not associated to any interventional device.

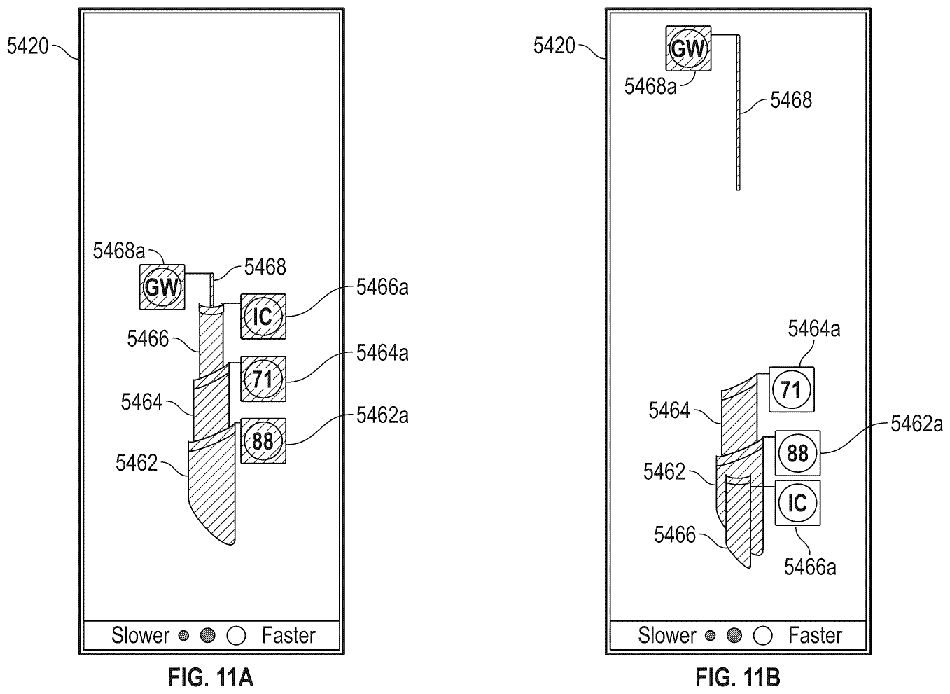

The instrument window 5420 can provide a visual indication of the axial position of the interventional device representations 5462, 5464, 5466 and 5468 with respect to each other. This can beneficially allow clinicians to assess the available range of motion for each interventional device associated with the interventional device representations 5462, 5464, 5466, 5468. As shown in FIG. 11A, the interventional device representations 5462, 5464, 5466, 5468 can be displayed so that a position of a distal end of each of the interventional device representations is shown. The visual indications 5462b, 5464b, 5466b, and 5468b of the interventional device representations 5462, 5464, 5466, 5468 can provide an indication of the position of the distal ends of the interventional devices corresponding to the interventional device representations with respect to each other. For example, the visual indication 5468b of the interventional device representation 5468 can provide an indication that the distal end of the interventional device associated with the interventional device representation 5468 (e.g., a guidewire) extends beyond a distal end of the interventional devices associated with the interventional device representations 5462, 5464 and/or 5466. The visual indications 5462b, 5464b, 5466b, and 5468b may provide an indication of relative distances between the interventional devices corresponding to the interventional device representations 5462, 5464, 5466, 5468 relative to each other. In some embodiments, as the interventional devices move relative to one another, the interventional device representations 5462, 5464, 5466, 5468 can move relative to one another so that the visual indications 5462b, 5464b, 5466b, and 5468b provide an indication of the relative positions of the distal ends of corresponding interventional devices as the interventional devices move. For example, as the guidewire associated to the interventional device representation 5468 is advanced though the vasculature of a patient, the position of the interventional device representation 5468 with respect to the interventional device representations 5464, 5466, 5468 may change to show the guidewire extended farther away from the interventional device representations 5464, 5466, 5468, as shown in FIG. 11B. In certain embodiments, information regarding the positions of the interventional devices and/or movement of the interventional devices (e.g., axial and/or rotational velocity, magnitude and/or direction of movement, etc.) may be received from one or more sensors (e.g., sensors 3135 and/or sensors 3130a) of a sensor system.

The instrument window 5420 can indicate whether an interventional device is positioned within another interventional device (e.g., in a nested configuration). In some embodiments, the interventional device representations 5462, 5464, 5466, 5468 can be shown in a transparent or semi-transparent appearance to allow other interventional device representations to be displayed and/or visualized through the semi-transparent interventional device representations. For example, as shown in FIG. 11B, the interventional device representation 5466 can be visualized through the interventional device representations 5462 and 5464 to indicate that the interventional device associated with the interventional device representation 5466 is positioned within the interventional devices associated with the interventional device representations 5462, 5464 but proximal to the distal ends of the interventional devices associated with the interventional device representations 5462.

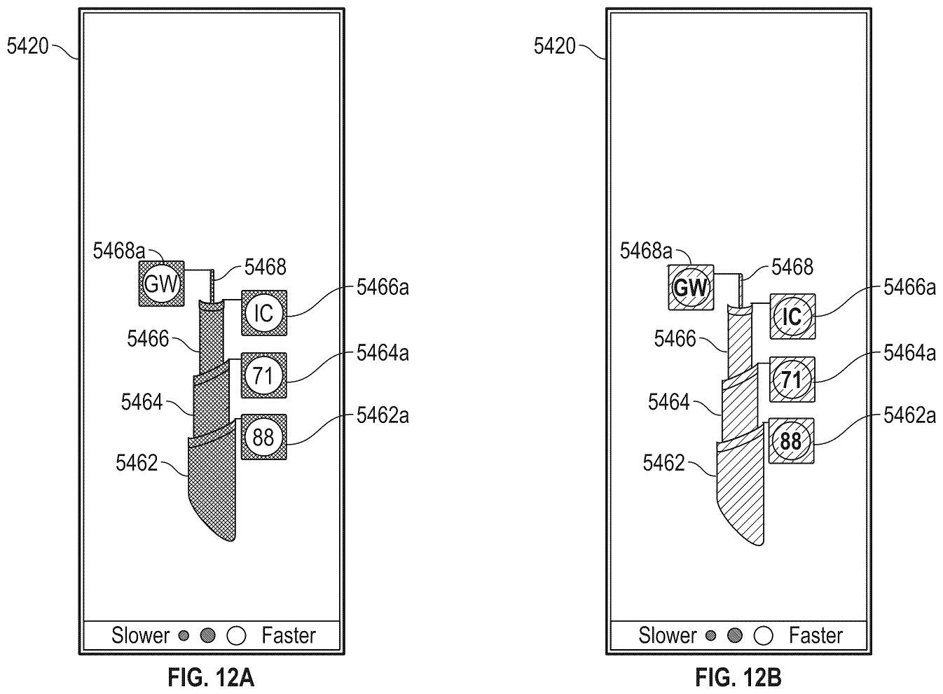

The interventional device representations 5462, 5464, 5466, 5468 and/or the interventional device markers 5462a, 5464a, 5466a, 5468a can provide an indication of whether the interventional devices associated to the interventional device representations 5462, 5464, 5466, 5468 are enabled. For instance, the interventional device representations 5462, 5464, 5466, 5468 and/or the interventional device markers 5462a, 5464a, 5466a, 5468a may be displayed in a first color (e.g., gray, transparent, etc.), shade (e.g., darker in comparison to when the devices are enabled), texture, pattern, etc. to indicate that the interventional devices associated to the interventional device representations 5462, 5464, 5466, 5468 are disabled, as shown in FIG. 12A. This can beneficially allow clinicians to easily identify which interventional devices are available and ready for use. The interventional device representations 5462, 5464, 5466, 5468 and/or the interventional device markers 5462a, 5464a, 5466a, 5468a can be displayed in a second color, shade (e.g., lighter in comparison to when the devices are disabled), texture, pattern, etc. to indicate that the interventional devices associated to the interventional device representations 5462, 5464, 5466, 5468 are enabled, as shown in FIG. 12B.

The instrument window 5420 can provide an indication of whether the interventional devices associated with the interventional device representations 5462, 5464, 5466, 5468 are selectively configured in a drive mode, which is also referred to herein as an operation mode, and/or stage mode. As described in relation to FIGS. 8A-8B, any of the control mechanisms described herein (e.g., the control mechanism 5300) can be configured to control two or more hubs and/or interventional devices simultaneously. For example, in a first drive mode, the hubs and/or interventional devices can be advanced to achieve supra-aortic access as described herein. In some embodiments, the instrument window 5420 can provide a visual indication of a current drive mode of the interventional devices. For example, in some embodiments, the instrument window 5420 can display all of the interventional device representations 5462, 5464, 5466, 5468 and/or all of the interventional device markers 5462a, 5464a, 5466a, 5468a with a particular visual appearance (e.g., color, shade, texture, pattern, etc.) to indicate that the interventional devices associated with the device representations 5462, 5464, 5466, 5468 are in a first drive mode (e.g., access mode). The instrument window 5420 can display one or more of the interventional device representations 5462, 5464, 5466, 5468 and/or the interventional device markers 5462a, 5464a, 5466a, 5468a with a different particular visual appearance to indicate that the interventional devices associated with one or more of the representations 5462, 5464, 5466, 5468 are in a second drive mode.

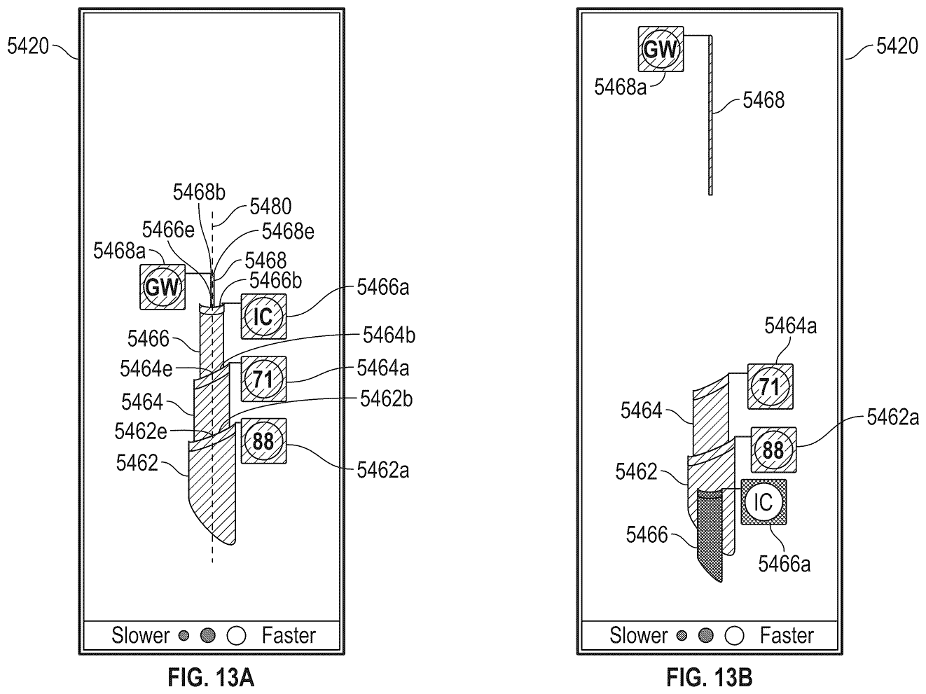

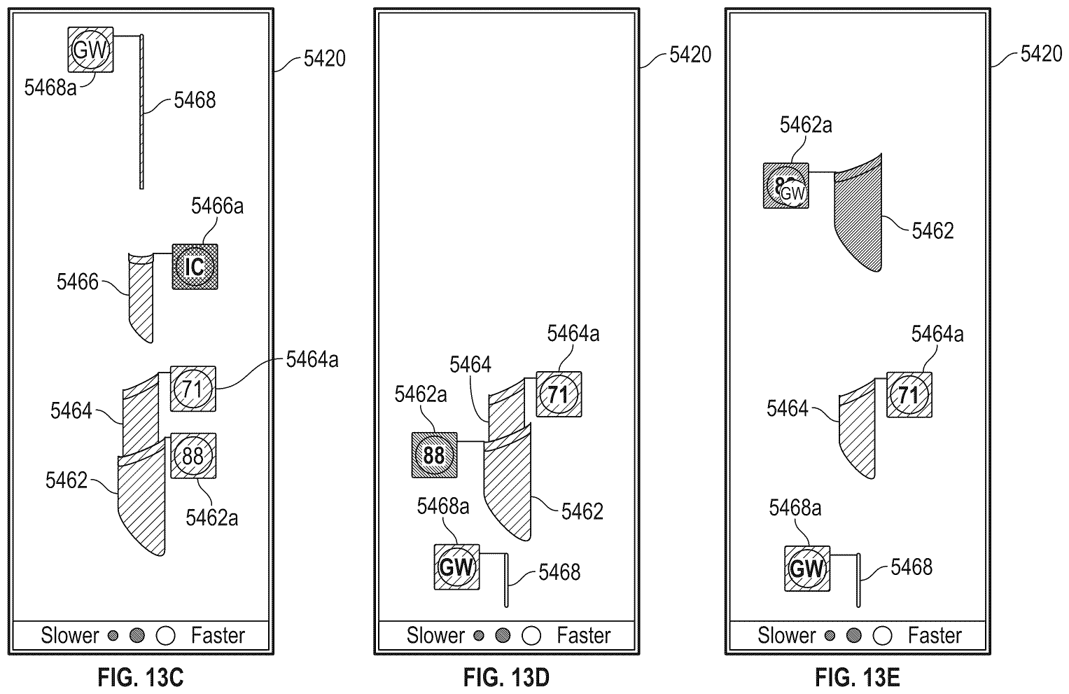

In certain embodiments, the instrument window 5420 can provide a visual indication of which devices are currently active (e.g., available for movement by one or more controls, such as joysticks 5332 and 5334) and which devices are inactive (e.g., not available for movement by one or more controls). Interventional devices may be inactive, for example, if they are not linked to be controlled by a control of a controller (e.g., joysticks 5332 and/or 5334), for example, in a particular drive mode or in response to a temporary linkage of particular interventional devices to the controls. In certain embodiments, the instrument window 5420 can display all active interventional device representations 5462, 5464, 5466, 5468 and/or all interventional device markers 5462a, 5464a, 5466a, 5468a in a first color, texture, pattern, etc. (as shown, for example, in FIG. 13A) and all inactive interventional device representations 5462, 5464, 5466, 5468 and/or all interventional device markers 5462a, 5464a, 5466a, 5468a in a second color, texture, pattern, etc. The first color, texture, and/or pattern, can be different than the second color, texture, and/or pattern. For example, as shown in FIG. 13B, the interventional device representation 5466 and/or the interventional device marker 5466a can be shown in a color, texture, pattern, different than that of the interventional device representations 5462, 5464, 5468 and/or all interventional device markers 5462a, 5464a, 5468a to indicate that the interventional device associated with the interventional device representation 5466 is not active. In some embodiments, by assessing which interventional devices are active and/or inactive, clinicians can beneficially identify whether the interventional devices associated with the interventional device representations 5462, 5464, 5466, 5468 are in a drive mode and/or what drive mode the interventional device representations 5462, 5464, 5466, 5468 are in. FIG. 13A may be an example of an instrument window 5420 during a first drive mode, as described herein, wherein a first control (e.g., joystick 5332) is linked to control a guidewire (corresponding to representation 5468), and a second control is linked to control an access catheter (corresponding to representation 5466), a procedure catheter (corresponding to representation 5464), and a guide catheter (corresponding to representation 5462). FIG. 13B may be an example of the instrument window 5420 during a second drive mode, as described herein, wherein a first control (e.g., joystick 5332) is linked to control a guidewire (corresponding to representation 5468), a second control (e.g., joystick 5334) is linked to control a procedure catheter (corresponding to representation 5464) and a guide catheter (corresponding to representation 5462), and neither the first control nor the second control is linked to control an access catheter (corresponding to representation 5466) such that the access catheter is considered inactive.

As shown in FIGS. 13C-13D, the instrument window 5420 can provide an indication of any active interventional device. Any of the interventional device markers 5462a, 5464a, 5466a, 5468a can provide a visual indication when the interventional device associated with the interventional device markers 5462a, 5464a, 5466a, 5468a is being driven or is not being driven. For example, the interventional device marker 5466a can include a color contrast and/or highlight to indicate that the interventional device (e.g., an insert catheter) associated with the marker 5466a is being driven, as shown in FIG. 13C. As another example, the interventional device marker 5462a can include a color contrast and/or highlight to indicate that the interventional device (e.g., 0.088 in. catheter; guide catheter 31) associated with the marker 5462a is being driven, as shown in FIG. 13D.

In some embodiments, one or more of the interventional device representations may change in appearance to indicate which interventional devices are being driven and/or which interventional devices are not being driven. For example, in certain embodiments, if an interventional device is being driven, the interventional device representation corresponding to the interventional device being driven can become brighter in appearance. In some embodiments, the interventional device representations corresponding to the interventional devices not being driven can become duller in appearance while an interventional device is being driven.

The interventional device representations 5462, 5464, 5466, 5468 can extend along a central longitudinal axis 5480, as shown in FIG. 13A. For instance, the interventional device representations 5462, 5464, 5466, 5468 can be centered along the central longitudinal axis 5480. The interventional device markers 5462a, 5464a, 5466a, 5468a can include at least two configurations and/or positions. In some cases, the interventional device markers 5462a, 5464a, 5466a, 5468a can extend away from the central longitudinal axis when the interventional devices associated with the interventional device representations 5462, 5464, 5466, 5468 are being driven. For example, the interventional device marker 5462a can move laterally or horizontally relative to the axis 5480 from a first position to a second position (e.g., further from the central axis 5480) to indicate that the interventional device associated with the interventional device representation 5462 is being driven.

The plurality of interventional device pointers 5462c, 5464c, 5466c, 5468c can additionally or alternatively provide a visual indication that the interventional devices associated with the interventional device representations 5462, 5464, 5466, 5468 are being driven. In some cases, the length of the plurality of interventional device pointers 5462c, 5464c, 5466c, 5468c can change from a first length to a second length to indicate whether an interventional device is being driven. Using the interventional device pointer 5462c as an example, a length of the pointer 5462c may transition from a first length to a second length larger than the first length to indicate that the interventional device associated with the interventional device representation 5462 and/or the interventional device marker 5462a is being drive. In some cases, the pointer 5462c may return to its original length when the interventional device is not being driven any more. The transition of the plurality of pointers 5462c, 5464c, 5466c, 5468c from a first state to a second state can beneficially allow clinicians to easily identify which interventional devices are being driven. Information regarding which interventional devices are being driven may be received from one or more sensors of a sensor system as described herein.

For example, the first position of the interventional device marker 5462a can be closer to the central longitudinal axis 5480 than the second position such that it is easily apparent to a user that the particular interventional device is in fact active and moving. In some instances, the interventional device representation corresponding to the driven interventional device can ungroup from the other representations. In some instances, the movement away from central axis improves a user's ability to operate the interventional device. For example, it can reduce errors and save time because a user doesn't have to think about or keep in their mind which interventional device are they moving. There might also not be a lot of available space on the user interface to indicate using text which device is moving. Accordingly, in some instances, the inventors realized that movement of the representations with respect to an axis can be used to convey important information about the status of operation.

The instrument window 5420 can provide an indication that two or more interventional devices associated with the interventional device representations 5462, 5464, 5466, 5468 are linked so that the two or more interventional devices move together in a same direction by a same magnitude and/or velocity regardless of whether the two or more interventional devices are linked to the same control of the controller. In some embodiments, when linked, two or more interventional devices can move simultaneously if the clinician moves at least one of the linked interventional devices using any control mechanism. In other embodiments, linked interventional devices can include a primary device and one or more secondary devices. Movement of a control to move the primary device can cause movement of the secondary device in the same direction by the same magnitude and/or velocity. Movement of a control to move the secondary device, but not the primary device, may not cause the primary device to move. Interventional devices may be so linked when controlled movements of one or more of the interventional devices would result in relative positions between the interventional devices, interventional device hubs, and/or hub adapters at distances greater than a total available separation distance (e.g., due to the length of the drive surface of the drive table or due to a length of a shuttle along which hub adapters translate) as described, for example, with respect to the table 8000. In some embodiments, movement of a control to move the secondary device, but not the primary device, in a direction that would increase a separation distance between the primary device and the secondary device may result in no movement of either the primary device or the secondary device. In some embodiments, movement of a control to move the secondary device, but not the primary device, in a direction that would decrease the separation distance between the primary device and the secondary device may cause the secondary device to move without movement of the primary device and/or may unlink the secondary device from the primary device.

As shown in FIG. 13E, an interventional device marker 5462a can include a first character set (e.g., number(s), letter(s) or combination thereof) and a second character set (e.g., number(s), letter(s), or combination thereof) to indicate that two interventional devices are linked to each other. For example, the interventional device marker 5462a can include a first character set (e.g., “88”) and a second character set (e.g., “GW”). The first character set can be associated with the interventional device (e.g., a guide catheter) associated with the interventional device representation 5462. As shown, the second character set (“GW”) is the same as the character (“GW”) shown by marker 5468a, indicating that the interventional device (e.g., a guidewire) associated with the representation 5468 associated with the marker 5468a is linked to the interventional device associated with the first character set (e.g., the guide catheter). As shown, the first character set may correspond to the primary interventional device. The second character set may correspond to the secondary interventional device. The character set for the primary interventional device may be larger than the character set for the secondary interventional device. In some embodiments, the character set for the primary interventional device can be positioned within the marker of the interventional device representation associated with the same interventional device as the character set for the primary interventional device. The character set for the secondary interventional device can be positioned in the same marker as the character se for the primary interventional device, but may be smaller in size or otherwise different in appearance to indicate that it is the character set for the secondary interventional device. Although first and second characters sets are described herein, in some embodiments, different symbols or identifiers may be used to identify the linked devices (e.g., primary and secondary devices).

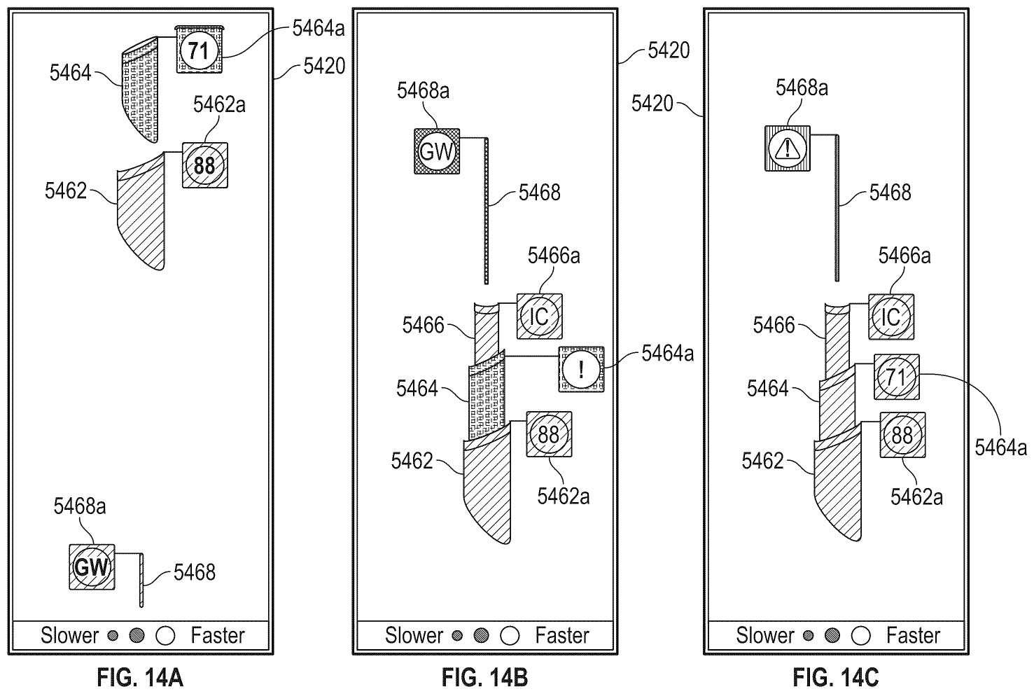

The instrument window 5420 can provide an indication about the status of the interventional devices associated with the interventional device representations 5462, 5464, 5466, 5468. In some cases, each of the interventional device markers 5462a, 5464a, 5466a, 5468a can be displayed in a color, texture, or pattern, etc. to indicate a status of the interventional device associated with the interventional device markers 5462a, 5464a, 5466a, 5468a. For example, the interventional device representation 5464 and/or the interventional device marker 5464a can transition from a first a first color (e.g., purple), texture, or pattern to a second color (e.g., yellow), texture, or pattern to indicate that the status of the interventional device associated with the interventional device representation 5464 and/or the interventional device marker 5464a has changed, as shown in FIG. 14A. In some embodiments, the status may indicate that the interventional device associated with the interventional device representation 5464 and/or the interventional device marker 5464a has reached a driving limit. An interventional device may reach its driving limit if the interventional device cannot move any further in a distal direction or a proximal direction. For example, an interventional device may reach its driving limit in a particular direction if further movement of the interventional device in that particular direction would cause a hub associated with that international device to collide with a hub of a second interventional device. An interventional device may reach a distal driving limit when the hub associated with the international device is positioned at a distal most point along a drive surface. An interventional device may reach a proximal driving limit when the hub associated with the interventional device is positioned at a proximal most point along a drive surface. An interventional device may reach a distal driving limit if a hub adapter associated with the interventional device is positioned at a distal most possible position within the drive table. An interventional device may reach a proximal driving limit if a hub adapter associated with the interventional device is positioned at a proximal most possible position within the drive table.

The interventional device markers 5462a, 5464a, 5466a, 5468a can display a symbol, character, number, or combination thereof to indicate the status of an interventional device. For example, as shown in FIG. 14B, the device marker 5464a can display an exclamation mark (“!”) in a first color (e.g., yellow), texture or pattern, to indicate a warning. In some cases, the warning may indicate that there is problem with the interventional device associated with the interventional device markers 5462a, 5464a, 5466a, 5468a (e.g., that the interventional device and/or hub associated with the interventional device may be experiencing a high force and/or resistance). As another example, and as shown in FIG. 14C, the device marker 5468a can display an exclamation mark (“!”) in a second color (e.g., red), texture, or pattern to indicate that there is an error with the interventional device associated with the interventional device markers 5462a, 5464a, 5466a, 5468a (e.g., that the interventional device has disconnected, is not working properly, etc.).

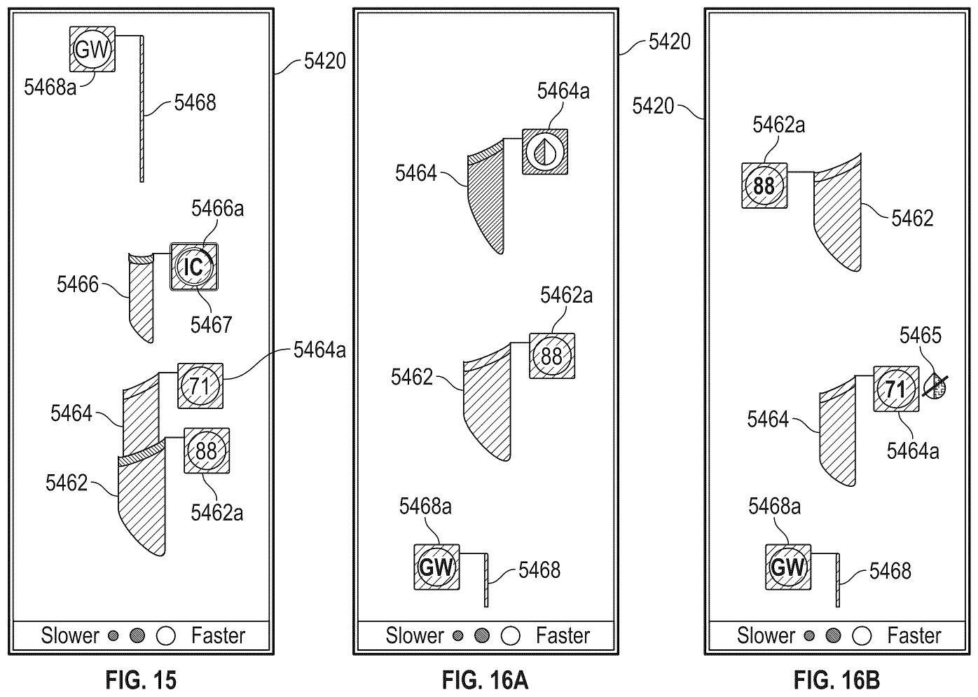

The interventional device representations 5462, 5464, 5466, 5468 and/or the interventional device markers 5462a, 5464a, 5466a, 5468a can include an indication about the rotational position or state of the interventional devices associated with the interventional device representations 5462, 5464, 5466, 5468. For example, as shown in FIG. 15, the interventional device marker 5466a can include a rotational indicator 5467 (e.g., a radial progress indicator). The rotational indicator 5467 can provide visual indication of a degree of rotation of an interventional device relative to a threshold of the interventional device about the longitudinal axis. For example, the threshold can represent a full revolution. In some embodiments, the rotational indicator can provide a visual indication of a direction of rotation. For example, in some embodiments, the rotational indicator may demonstrate clockwise movement to indicate clockwise rotation of a corresponding interventional device and counterclockwise movement to demonstrate counterclockwise movement of the corresponding interventional device.

The rotational indicator 5467 can be displayed inside the interventional device marker 5466a. In some cases, the rotational indicator 5467 can include a ring or circular shaped progress bar, but the rotational indicator 5467 can include other shapes (e.g., triangular, square, etc.). When the interventional device associated with the interventional device representation 5466 is rotated (e.g., the rotational position of the interventional device is changed), the rotational indicator 5467 (e.g., progress bar) may start to fill to indicate how much the interventional device has been rotated. In some cases, the rotational indicator 5467 can fill in its entirety to indicate that the interventional device has been rotated one full revolution. Upon completing a full revolution, the rotational indicator 5467 may reset. In some cases, the rotational indicator 5467 may fill in a clockwise direction and/in a counterclockwise direction to indicate which rotational direction the interventional device is moving in.

The ring shaped progress bar of the rotational indicator 5467 can be filled to indicate the degree and/or direction of rotation of an interventional device. For example, in some embodiments, the ring shaped progress bar of the rotational indicator 5467 can be filled by about 25% in a clockwise direction to indicate that the interventional device has been rotated, from a starting position, one quarter of a revolution in a clockwise direction. As another example, the ring shaped progress bar of the rotational indicator 5467 can be filled by about 50% in a counterclockwise direction to provide a visual indication that the interventional device has been rotated, from a starting position, half a revolution in a counterclockwise direction. The ring shaped progress bar of the rotational indicator 5467 may reset (e.g., be empty and/or shown at 0%) when the interventional device completes a full revolution.

The instrument window 5420 can provide an indication of whether contrast injection, and/or other fluid delivery is being delivered via the interventional devices associated with the interventional device representations 5462, 5464, 5466, 5468. For example, the interventional device representations 5462, 5464, 5466, 5468 and/or the interventional device markers 5462a, 5464a, 5466a, 5468a may change from a first color (e.g., purple), texture, or pattern to a second color (e.g., blue), texture or pattern to indicate that contract injection and/or other fluid delivery (e.g., saline) is active in one or more of the interventional devices. As shown, in FIG. 16A, the interventional device marker 5464a can display a symbol (e.g., a droplet) to indicate that the interventional device associated with the interventional device marker 5464a is delivering contrast. In some cases, the contrast symbol in the interventional device marker 5464a can replace the symbol, number, and or character that indicates the interventional device associated with the interventional device marker 5464a. The instrument window 5420 can also provide an indication of whether contrast is unavailable. For example, the instrument window 5420 can display a symbol 5465, as shown in FIG. 16B. The symbol 5465 can be displayed next to an interventional device marker (e.g., interventional device marker 5464a). The symbol 5465 can provide an indication that contrast is unavailable for the interventional device associated with the interventional device marker where the symbol is displayed. The same or additional symbols may be used to provide information regarding the delivery of other fluids.

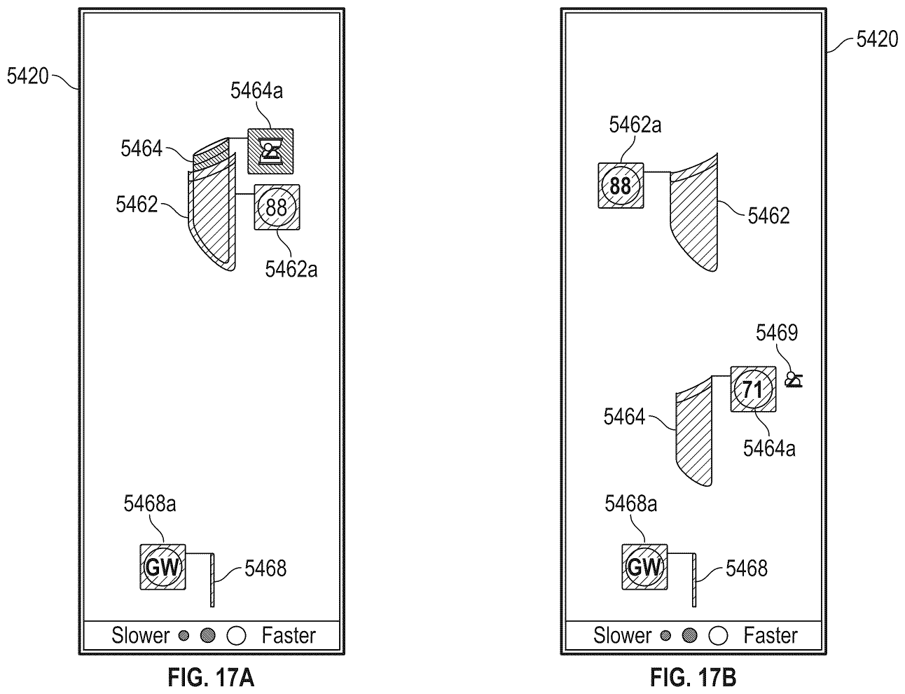

The instrument window 5420 can provide an indication of whether aspiration is being applied via the interventional devices associated with the interventional device representations 5462, 5464, 5466, 5468. For example, the interventional device representations 5462, 5464, 5466, 5468 and/or the interventional device markers 5462a, 5464a, 5466a, 5468a may change from a first color (e.g., purple), texture, or pattern to a second color (e.g., green), texture or pattern to indicate that aspiration is active and/or available in one or more of the interventional devices. As shown, in FIG. 17A, the interventional device marker 5464a can display a symbol (e.g., an aspiration icon) to indicate that the interventional device associated with the interventional device marker 5464a is applying aspiration and/or that aspiration is available in that interventional device. In some cases, the aspiration symbol in the interventional device marker 5464a can replace the symbol, number, and or character that indicates the interventional device associated with the interventional device marker 5464a. The instrument window 5420 can also provide an indication of whether aspiration is unavailable. For example, the instrument window 5420 can display a symbol 5469, as shown in FIG. 17B. The symbol 5469 can be displayed next to an interventional device marker (e.g., interventional device marker 5464a). The symbol 5469 can provide an indication that aspiration is unavailable for the interventional device associated with the interventional device marker where the symbol is displayed.

Information regarding fluidics (e.g., the status and/or availability of contrast injection, other fluid delivery, and/or aspiration) may be received from a sensor system including one or more sensors (e.g., in the fluidics system, interventional devices, hubs, drive table, etc.).

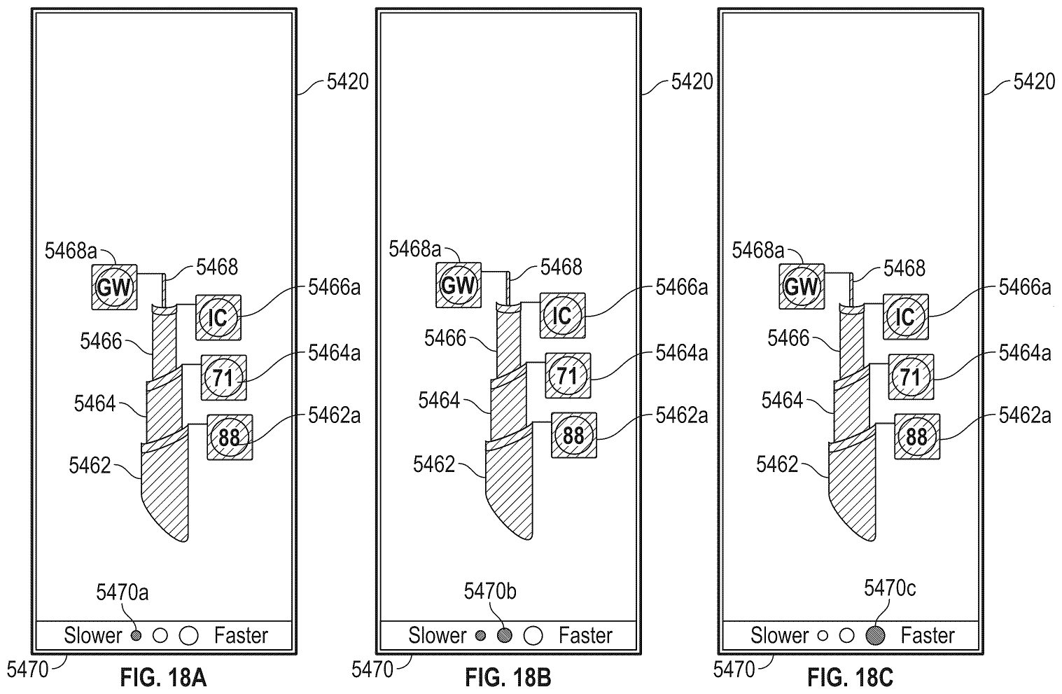

In cases where the interventional devices associated with the interventional device representations 5462, 5464, 5466, 5468 and/or the interventional device markers 5462a, 5464a, 5466a, 5468a can be operated at different speeds, the instrument window 5420 can provide an indication of the speed setting the interventional devices. For example, as shown in FIGS. 18A-18C, the instrument window 5420 can include a speed indicator 5470. The speed indicator 5470 can include one or more speed markers. In some cases, the speed indicator 5470 can include three speed markers, 5470a, 5470b, 5470c. The speed markers, 5470a, 5470b, 5470c can provide an indication about the speed at which the interventional devices associated with the interventional device representations 5462, 5464, 5466, 5468 are being operated. Each of the speed markers 5470a, 5470b, 5470c can be associated to a range of speeds (slow, medium, fast, etc.) over which the interventional device may be moved (axially and/or rotationally, in response to movement of a control (e.g., joystick 5332 or joystick 5334) of a controller (e.g., controller 5310).

The position of the interventional device markers 5462a, 5464a, 5466a, 5468a relative to the central longitudinal axis 5480 can provide an indication about which button, joystick, etc. of a control mechanism (e.g., control mechanism 5300) is controlling the interventional device associated with the interventional device markers 5462a, 5464a, 5466a, 5468a. In some embodiments, the position of the button, joystick, etc. (e.g., the first joystick 5332) relative to the central axis 5380 of the control mechanism 5300 can correspond to the positioning of the interventional device marker relative to the central longitudinal axis 5480 on the instrument window 5420. For example, as shown in FIGS. 18A-18C, the interventional device marker 5468a can be displayed left of the interventional device representation 5468 and the central longitudinal axis 5480 to indicate that the interventional device associated with the interventional device marker 5468a is being controlled and/or can be controlled by a left joystick (e.g., the first joystick 5332) of a control mechanism (e.g., handheld controller 5310). As another example, the interventional device markers 5462a, 5464a, 5466a can be displayed right of the interventional device representations 5462, 5464, 5466 to indicate that the interventional devices associated with the interventional device markers 5462a, 5464a, 5466a are being controlled and/or can be controlled by a right joystick (e.g., the second joystick 5334) of a control mechanism (e.g., handheld controller 5310). In some embodiments, the controller and user interface can be configured such that if a central axis of the controller (e.g., axis 5380) is aligned with a central axis of the interventional device representations (e.g., axis 5480), at least some movement of the joysticks can cause the interventional device representations to move in the same direction as the joysticks on the user interface. For example, forward or rearward movement of one of the joysticks can result in movement of the corresponding representations (due to movement of the corresponding interventional devices) in the same direction that the user moves the joystick. For example, with respect to FIG. 8A, upward movement of the joystick 5332 along the axis A5 towards the button 5352 can cause a corresponding interventional device representation to move upward along the axis 5480 (e.g., towards the notification window 5430 in FIG. 9).

In some embodiments, as described herein, a user can operate a controller (e.g., controller 5310) to link an interventional device with a particular control of the controller (e.g., joystick 5332 or joystick 5334), for example, to temporarily change which interventional devices are controlled by which controls in a particular drive mode. In some embodiments, if a particular interventional device is linked to one of a first control and a second control, and is then subsequently linked to the other of the first control and the second control, the interventional device marker and/or pointer corresponding to the particular interventional device may switch from one side of the central axis 5480 to the other side of the central axis 5480. This change in appearance may make it easier for a user to see that a linkage has occurred.

In some embodiments, at the time a particular interventional device becomes linked with a particular control (e.g., in response to a user input), the interventional device representation, interventional device marker, and/or pointer corresponding to the particular interventional device may change in appearance (e.g., shape, pattern, texture, position, etc.) to indicate that the particular interventional device is linked to the particular control. For example, in certain embodiments, any of the same visual indications to indicate that an interventional device is currently being drive may (at least temporarily) be provided to indicate the link of the particular interventional device to the particular control. This change in appearance may make it easier for a user to see that a linkage has occurred.

For example, as described herein, FIG. 13A may be an example of an instrument window 5420 during a first drive mode, wherein a first control (e.g., joystick 5332) is linked to control a guidewire (corresponding to representation 5468), and a second control is linked to control an access catheter (corresponding to representation 5466), a procedure catheter (corresponding to representation 5464), and a guide catheter (corresponding to representation 5462). In response to a user input (e.g., actuation of a button 5352 of controller 5310), the guide catheter may be temporarily linked to the first control (e.g., joystick 5332). At the time the guide catheter is linked to the first control, the interventional device marker 5462a may move from the side of the axis 5480 associated with the second control (e.g., the right side of the central axis 5480 as shown in FIG. 13A) to the side of the axis 5480 associate with the first control (e.g., the left side of the central axis 5480). Additionally, in some embodiments, one or more of the marker 5462a, representation 5462, and pointer 5462c may change in appearance, at least temporarily, at the time the guide catheter becomes linked to the first control. The changes in appearance may be the same as or similar to any of the change in appearance that takes place when the guide catheter is driven. For example, the marker 5462a and or representation 5462 may change in appearance (e.g., including a color contrast or highlight relative to the other markers and/or representations) to indicate the linkage of the first control. In some embodiments, the other representations 5464, 5466, and 5468 and/or other markers 5464a, 5466a, and 5468a may additionally or alternatively change in appearance (e.g., including a color contrast (for example to be duller in color than representation 5462 and/or marker 5462a) or highlight relative to the representation 5462 and/or marker 5462a) to indicate the linkage of the first control. In some embodiments, the marker 5462a may move laterally away from the central axis 5480 to indicate the linkage of the guide catheter to the first control. In some embodiments, the pointer 5462c can extend or elongate between the representation 5462 and the marker 5462a to indicate the linkage of the guide catheter to the first control.

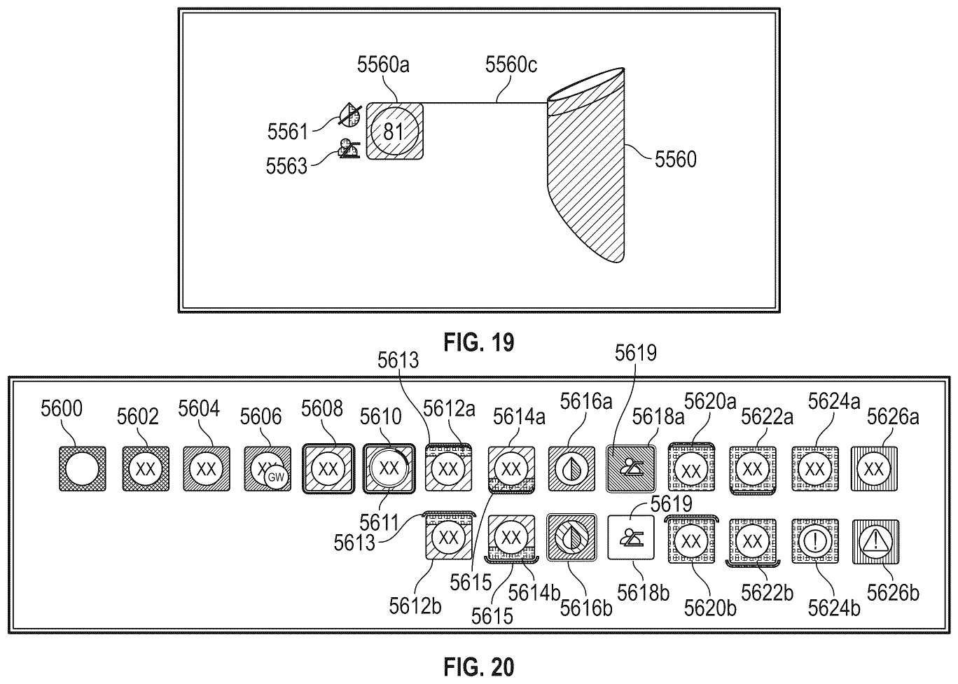

FIG. 19 shows another example of an interventional device representation 5560, an interventional device marker 5560a, an interventional device pointer 5560c, and symbols 5561, 5563 that can be displayed on the instrument window 5420. As previously described, the interventional device marker 5560a can provide an indication about the interventional device associated with the interventional device representation 5560. For example, as shown in FIG. 19, the interventional device marker 5560a can provide an indication that the interventional device representation 5560 is associated (e.g., linked) with a catheter (e.g., a 0.081 French catheter). The symbols 5561 and 5563 can provide an indication about the status of aspiration, contrast injection and/or other fluid delivery. For example, as shown in FIG. 19, the symbol 5561 can provide an indication that injection, and/or fluid delivery is disabled and/or unavailable. The symbol 5563 can provide an indication that aspiration is disabled and/or unavailable.

FIG. 20 shows examples of interventional device markers or appearances of interventional device markers that can be displayed on the instrument window 5420. The appearance of the interventional device markers can change to indicate a change in the status of the interventional devices associated with the interventional device markers. For example, one or more of the interventional device markers described herein with respect to FIGS. 10A-19 may change in appearance to include the features of the markers shown in FIG. 20 to indicate a change in the status of the interventional devices associated with the interventional device markers. In some embodiments, any of the interventional device marker appearances shown in FIG. 20 can include a first animation state and a second animation state. The interventional device markers can transition from the first animation state to the second animation state to provide a visual indication of a change in the operation status of the interventional device associated with the interventional device markers. In some embodiments, the interventional device markers can include a first color (e.g., gray, transparent, purple, blue, green, yellow, red, etc.), texture, or pattern to indicate the status of the interventional device associated with the interventional device marker. The interventional device markers can also display a character set, symbol, and/or combination thereof to provide an indication of the interventional device associated with the interventional device markers and/or to provide an indication of the operational mode of the interventional device (e.g., active, inactive, enabled, disabled, aspiration on/off, fluid delivery on/off, etc.).

In some cases, an interventional device marker 5600 can be shown in a first color (e.g., gray), texture, or pattern to indicate that the interventional device associated with the interventional device marker 5600 is disabled. The interventional device marker 5600 may not include a character set, symbol, etc. to indicate that an interventional device is not associated (e.g., linked) with the interventional device marker 5600. Upon installation of an interventional device, the interventional device marker can display one or more character sets, symbols, etc. to provide an indication about the interventional device installed (e.g., as represented by “XX” in interventional device marker 5602). An interventional device marker 5604 can be shown in a second color (e.g., light purple), texture, or pattern to indicate that the interventional device associated with the interventional device marker 5604 is enabled. An interventional device marker 5606 can include a first character set or symbol (e.g., represented by “XX” in the interventional device marker 5606) and a second character set (e.g., “GW”) to indicate that a first interventional device (e.g., a guide catheter and a second interventional device (e.g., a guidewire) are linked to each other.

An interventional device marker 5608 can be shown in a color (e.g., dark purple), texture, or pattern to indicate that the interventional device associated with the interventional device marker 5608 is being driven (e.g., being moved). As described in relation to FIG. 15, an interventional device marker 5610 can include a rotational indicator 5611 displayed inside the interventional device marker 5610. The rotational indicator 5611 can provide an indication about the rotational position of the interventional device associated with the interventional device marker 5610. For example, the rotational indicator 5611 can include a ring shaped progress bar. As the interventional device associated with the interventional device marker 5610 is rotated, the ring shaped progress bar may start filling in a clockwise and/or counterclockwise directions to provide a visual indication about the direction in which the interventional device has rotated and/or the degree of rotational motion of the interventional device. For example, and as shown by the interventional device marker 5610, the ring shaped progress bar of the rotational indicator 5611 can be filled by about 25% in a clockwise direction to indicate that the interventional device has been rotated, from a starting position, one quarter of a revolution in a clockwise direction. As another example, the ring shaped progress bar of the rotational indicator can be filled by about 50% in a counterclockwise direction to provide a visual indication that the interventional device has been rotated, from a starting position, half a revolution in a counterclockwise direction. The ring shaped progress bar of the rotational indicator 5611 may reset (e.g., be empty) when the interventional device complete a full revolution.

Any of the interventional device markers described herein can provide an alert when the interventional device associated with the interventional device marker (and/or the hub associated with the interventional device) is nearing a proximal end and/or a distal end of their motion range. In some embodiments, the appearance of the interventional device markers can change to indicate a particular state and/or status of the interventional device. An interventional device marker can transition from a first animation state to a second animation state to provide a visual indication about the status and/or operation state of the interventional device associated with an interventional device marker and/or interventional device representation. For example, in a first animation state, an interventional device marker 5612a can display a color (e.g., yellow), texture, or pattern on a top portion of the interventional device marker 5612a to indicate that that the interventional device associated with the interventional device marker 5612a is nearing a distal drive limit (e.g., nearing a maximum allowable distal position or nearing a hub positioned distally to the hub of the associated interventional device). When the interventional device associated with the interventional device marker 5612a gets closer to the distal drive limit, the interventional device marker 5612a can transition to a second animation state, as represented by the interventional device marker 5612b.

In the second animation state, a portion of the second interventional device marker 5612b can be different than the interventional device marker 5612a. For example, an indicator 5613 along a top portion of the interventional device marker 5612b can extend out of the top edge of the interventional device marker 5612b. In the first animation state, the indicator 5613 can be shown bounded within the top edge of the of the interventional device marker 5612a. The transition of the interventional device markers from the first animation state to the second animation state can be progressive, as opposed to just switching from the interventional device marker 5612a to the interventional device marker 5612b. This can beneficially allow clinicians to assess how far or close the interventional device associated with the interventional device markers 5612a, 5612b is from the distal drive limit.

The interventional device markers can also provide an indication of whether the interventional device (and/or the hub associated with the interventional device) associated with an interventional device marker is nearing a proximal drive limit (e.g., nearing a maximum allowable proximal position or nearing a hub positioned proximally to the hub of the associated interventional device). In a first animation state, an interventional device marker 5614a can display a color (e.g., yellow), texture, or pattern on a bottom portion of the interventional device marker 5614a to indicate that that the interventional device associated with the interventional device marker 5614a is nearing a proximal drive limit. When the interventional device associated with the interventional device marker 5614a gets closer to the proximal drive limit, the interventional device marker 5614a can transition to a second animation state, as represented by the interventional device marker 5614b.

In the second animation state, a portion of the second interventional device marker 5614b can be different than the interventional device marker 5614a. For example, an indicator 5615 along a bottom portion of the interventional device marker 5614b can extend out of the bottom edge of the interventional device marker 5614b. In the first animation state, the indicator 5615 can be shown bounded within the bottom edge of the of the interventional device marker 5614a. The transition of the interventional device markers from the first animation state to the second animation state can be progressive, as opposed to just switching from the interventional device marker 5614a to the interventional device marker 5614b. This can beneficially allow clinicians to assess how far or close the interventional device associated with the interventional device markers 5614a, 5614b is from the proximal drive limit.

In some cases, any of the interventional device markers described herein can be shown in a color (e.g., blue), texture, or pattern to indicate that contrast injection and/or other fluid delivery is available and/or active at the interventional device associated with the interventional device markers. In some cases, the interventional device markers can transition from a first animation state to a second animation state to provide a visual indication about the status and/or operation state of the interventional device and/or the application of contrast injection and/or other fluid delivery. For example, in a first animation state, an interventional device marker 5616a can include a symbol and/or a picture (e.g., a water drop) to indicate, for example, that contrast injection and/or other fluid delivery is available at the interventional device associated with the interventional device marker 5616a. In a second animation state, the interventional device marker 5616b can include a color contrast and/or highlight around its edge to indicate that contrast injection and/or other fluid delivery is active on the interventional device associated with the interventional device marker 5616b.

In some cases, the interventional device markers can show an action being undertaken by an interventional device and/or the progression of the action. An interventional device marker 5618a can be shown in a color (e.g., green), texture, or pattern to indicate that aspiration is available and/or active at the interventional device associated with the interventional device marker 5618a. The interventional device markers can transition from a first animation state to a second animation state to provide a visual indication about the status of aspiration in the interventional devices associated with the interventional device markers. For example, in a first animation state, the interventional device marker 5618a can include an aspiration indicator 5619. The aspiration indicator 5619 can provide a visual indication about the amount of aspiration time available in the interventional device associated with the interventional device marker 5618a. In the first animation state, the aspiration indicator 5619 can be represented by a bar in a full position, as shown by the interventional device marker 5618a. As aspiration is applied and the availability of aspiration decreases, a height of the of the bar of the aspiration indicator 5619 may start decreasing until aspiration at the interventional device is no longer available. When aspiration is no longer available and/or expired, the interventional device marker 5618a can transition to a second animation state, as represented by the interventional device marker 5618b. In the second animation state, the bar of the aspiration indicator may disappear to indicate that aspiration is not available at the aspiration catheter associated with the interventional device marker 5618b. As aspiration is restored in the interventional device, the bar of the aspiration indicator 5619 may transition from the first animation state to a second animation state, to provide an indication that aspiration at the interventional has been restored. In some cases, the interventional device marker 5618a can include a color contrast and/or highlight around its edge to indicate that aspiration is active on the interventional device associated with the marker 5618a.

The interventional device markers described herein can provide an alert when the interventional devices (and/or the hubs associated with the interventional devices) associated with the interventional device markers have reached a proximal drive limit and/or a distal drive limit. In some cases, the interventional device markers can include two or more animations states to indicate an event and/or action being undertaken by the interventional device. For example, in a first animation state, an interventional device marker 5620a can display a color (e.g., yellow), texture, or pattern on a top portion of the interventional device marker 5620a to indicate that that the interventional device associated with the interventional device marker 5620a has reached a distal drive limit (e.g., cannot be advanced any more). In a second state, at least a portion of the interventional device marker 5620b can extend outside a top edge of the interventional device marker 5620b when a clinician attempts to extend the interventional device beyond the distal drive limit of the interventional device (e.g., after the first animation state has already been displayed).

The interventional device markers can transition from a first animation state to a second animation state to indicate that the interventional devices have reached a proximal drive limit. For example, in a first animation state, an interventional device marker 5622a can display a color (e.g., yellow), texture, or pattern on a bottom portion of the interventional device marker 5622a to indicate that that the interventional device associated with the interventional device marker 5622a has reached a proximal drive limit (e.g., cannot be retracted any more). In a second state, at least a portion of the interventional device marker 5622b can extend outside a bottom edge of the interventional device marker 5622b when a clinician attempts to retract the interventional device beyond the proximal drive limit of the interventional device (e.g., after the first animation state has already been displayed).

In some cases, in a first animation state, an interventional device marker 5624a can be shown in a color (e.g., yellow), texture, or pattern to provide a warning about the device associated with the interventional device marker 5624a (e.g., that the interventional device and/or hub associated with the interventional device may be experiencing a high force and/or resistance). In a second animation state, an interventional device marker 5624b can also display a warning sign. In a first animation state, an interventional device marker 5626a can be shown in a color (e.g., red), texture, or pattern to indicate that there is an error with the device associated with the interventional device marker 5626a (e.g., that the interventional device has disconnected, is not working properly, etc.). In a second state, an interventional device marker 5626b can display can also display a warning sign.

While the appearances of the markers in FIG. 20 are described with respect to examples of different markers, one of skill in the art would understand that any of the markers described herein may change appearance to include features of any of the markers described in FIG. 20 to provide information to a user regarding the status of the interventional devices and/or other features of the drive system.

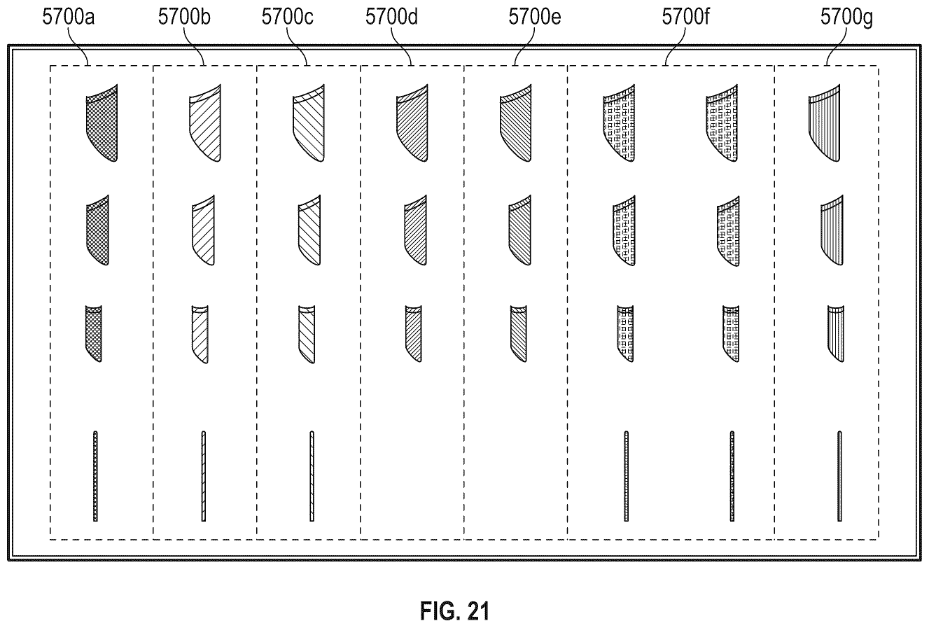

FIG. 21 shows examples of interventional device representations that can be displayed on the instrument window 5420. The interventional device representation can include a first color (e.g., gray, transparent, purple, blue, green, yellow, red, etc.), texture, or pattern to indicate the status of the interventional device associated with the interventional device representation.

In some cases, the interventional device representation 5700a can be shown in a first color (e.g., gray), texture, or pattern to indicate that the interventional device associated with the interventional device representation 5700a is disabled. The interventional device representation 5700b can be shown in a second color (e.g., light purple), texture, or pattern to indicate that the interventional device associated with the interventional device representation 5700b is enabled. The interventional device representation 5700c can be shown in a third color (e.g., dark purple), texture, or pattern to indicate that the interventional device associated with the interventional device representation 5700c is being driven (e.g., being moved). In some cases, the interventional device representation 5700d can be shown in a fourth color (e.g., blue), texture, or pattern to indicate that contrast injection and/or other fluid delivery is available and/or active at the interventional device associated with the interventional device representation 5700d. The interventional device representation 5700e can be shown in a fifth color (e.g., green), texture, or pattern to indicate that aspiration is available and/or active at the interventional device associated with the interventional device representation 5700e. In some cases, the interventional device representation 5700f can be shown in a sixth color (e.g., yellow), texture, or pattern to provide a warning about the device associated with the interventional device representation 5700f (e.g., that the interventional device and/or puck associated with the interventional device may be experiencing a high force and/or resistance). The interventional device representation 5700g can be shown in a seventh color (e.g., red), texture, or pattern to indicate that there is an error with the device associated with the interventional device representation 5700g (e.g., that the interventional device has disconnected, is not working properly, etc.).

Any of the embodiments of controllers and control systems described herein can be used to control a telescoping drive table and/or a drive table configured to move axially to cause axial movement of a hub adapter coupled to the drive table. For example, in certain embodiments, when a control (e.g., joystick 5332 and/or joystick 5334) is actuated to cause a corresponding axial movement of a particular hub and/or interventional device coupled to that hub, the corresponding axial movement of the particular hub and/or interventional device can be effectuated by an axial movement of the drive table itself or a component within the drive table to which one or more hub adapters are coupled, alternatively or in addition to axial movement of the hub adapter to which the particular hub and/or interventional device is coupled along the drive table or component within the drive table.

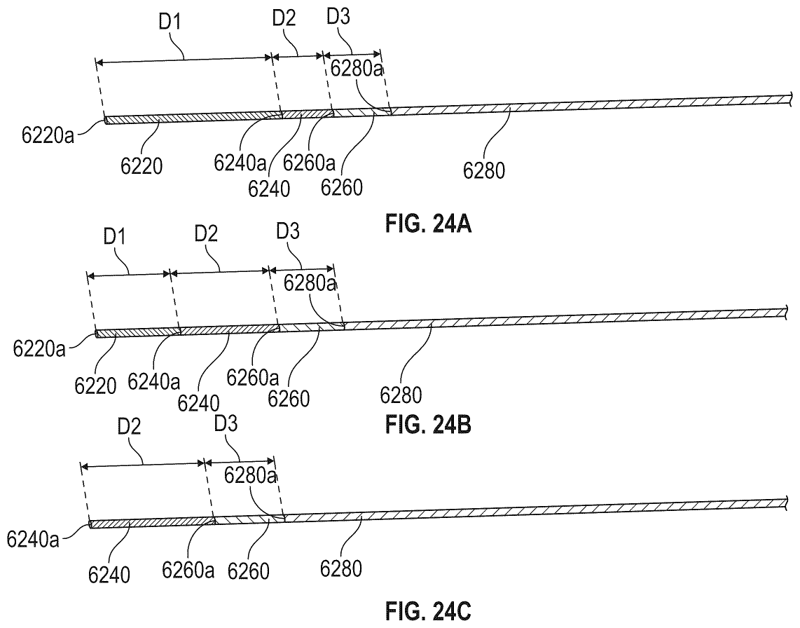

FIGS. 24A-24C illustrate an example of a drive mode for a control mechanism (e.g., a handheld controller). The drive mode illustrated in FIGS. 24A-24C, which may also referred herein to as auto stacking, can be applied to and/or otherwise be incorporated into any of the control mechanisms described herein and in connection with any of the respective hubs and/or interventional devices described herein. The drive mode can be used with a control mechanism to control at least one interventional device. For example, the drive mode may control a first interventional device 6220, a second interventional device 6240, a third interventional device 6260, and/or a fourth interventional device 6280. The first interventional device 6220 can be a guidewire. The second interventional device 6240 can be a catheter (such as an access catheter). The third interventional device 6260 can be a catheter (such as a procedure catheter). The fourth interventional device 6280 can include be catheter (such as guide catheter). As described herein, the interventional devices may be concentrically nested and coupled to interventional device hub assemblies.

As described herein, one or more of the interventional devices can be axially translated inside another interventional device. For example, the first interventional device 6220 can be axially translated inside the second, third, and fourth interventional devices 6240, 6260, 6280. The second interventional device 6240 can be axially translated inside the third and fourth interventional devices 6260, 6280. The third interventional device 6260 can be axially translated inside the fourth interventional device 6280. One or more of the interventional devices may be translated simultaneously (e.g., at the same speed or at different speeds) or individually. One or more of the interventional devices may be rotated simultaneously (e.g., at the same speed or at different speeds) or individually.

In certain embodiments, in an auto stacking drive mode, two or more devices may be moved (e.g., via one or more control algorithms) in response to actuation of a control of a controller to achieve a desired configuration (e.g., a desired set of relative positions of interventional devices relative to one another). For example, the two or more devices may be moved at different velocities in response to actuation of a single control of a controller in order to reach a desired configuration. In some embodiments, the two or more devices may be moved by different amounts in response to actuation of a single control of a controller in order to reach a desired configuration.

In some embodiments, the auto stacking drive mode can be applied to all of the devices within an interventional device assembly or to a subset of the devices of the interventional device assembly.

In some embodiments, the desired configuration may relate to a desired overall stiffness profile of the interventional device assembly or a subset of the interventional device assembly. An interventional device assembly or stack or subset thereof having multiple concentrically nested interventional devices can have an overall stiffness profile that depends on the relative positions of the concentrically nested interventional devices. For example, the stack may have a different stiffness at a first point along the length of the stack at which only an access catheter is present (e.g., because a guide catheter, procedure catheter, and/or guidewire are all positioned proximally to the point) than at a second point where both an access and procedure catheter are present. Similarly, each of the individual interventional devices may have stiffness profiles that vary along their length. Accordingly, a first point or segment along an interventional device stack at which a proximal segment of a procedure catheter and a proximal segment of an access catheter are located may have a different local stiffness than a second point or segment along the interventional device stack at which a distal segment of the procedure catheter and a distal segment of the access catheter are located. In some embodiments, a stiffness profile can characterize local stiffnesses at a plurality of points or segments along a length of an interventional device or interventional device stack and/or rates of stiffness change along a length of an interventional device or interventional device stack.

In some embodiments, interventional device stiffness or flexibility or interventional device stack stiffness or flexibility can be determined using the cantilever beam test. The cantilever beam test can be done along the length of an interventional device or interventional device stack to characterize the local segment stiffness and the rate of stiffness change. For example, in certain embodiments, the peak load may be measured at one or more increments along an interventional device or interventional device stack. In some embodiments, an interventional device of interventional device stack can be secured at a 5 mm distance from a target location to be measured. A force can then be applied to cause a 4 mm displacement at the target location of the interventional device or interventional device stack. The amount of force applied is measured at each sampling location (e.g., every 6 mm) to create a flexibility profile for the interventional device or interventional device stack. For example, for each sampling location, a peak load can be measured during the application of force to cause a 4 mm displacement. The stiffness profile can illustrate the peak load (gF) experienced along each sampling location (e.g., every 6 mm).

In some embodiments, it may be possible to provide a desired overall stack stiffness profile and/or one or more desired local stiffnesses or stiffness profiles at particular points or segments along the length of the interventional device stack by adjusting the relative positions of the interventional devices within the stack, for example, via one or more control algorithms. In some embodiments, the desired overall stack stiffness profile and/or desired local stiffnesses or stiffness profiles of the stack may change as the stack is advanced into different portions of the anatomy (e.g., the aortic arch, the procedure site, etc.). In some embodiments, the positions of the interventional devices can be changed to adjust the overall stiffness profile of the stack and/or local stiffnesses or stiffness profiles of the stack by adjusting the velocities of the individual interventional devices to achieve particular relative positions between the interventional devices of the stack. In some embodiments, it may be desirable to maintain a leading (e.g., distally most positioned) interventional device at a velocity commanded by operation of a control mechanism as described herein. In such embodiments, the velocities of only the trailing (e.g., more proximally positioned) interventional devices can be changed (e.g., to achieve particular relative positions between the interventional devices of the stack) to adjust the overall stiffness profile of the stack and/or local stiffnesses or stiffness profiles of the stack.

Each of the interventional devices can include a distal end. For example, the first interventional device 6220 can include a distal end 6220a, the second interventional device 6240 can include a distal end 6240a, the third interventional device 6260 can include a distal end 6260a, and the fourth interventional device 6280 can include a distal end 6280a. During a procedure, it may be desirable to maintain the distal ends of the interventional devices within a predefined distance from each other (for example, when a user actuates an input, such as joystick, to move multiple of the interventional devices) or to move the interventional devices so that their distal ends will arrive at predefined distances from one another at particular sections of the anatomy or during different portions of a procedure. For example, distal ends 6220a and 6240a can be separated by a distance D1, distal ends 6240a and 6260a can be separated by a distance D2, and distal ends 6260a and 6280a can be separated by a distance D3. For example, there may be a desired configuration of the distal ends of two or more of the interventional devices during a procedure or multiple different desired configurations at different portions of a procedure. For example, in some embodiments, it may be desirable to maintain the distal ends of two or more of the interventional devices within predefined distances or to move the interventional devices so that their distal ends will arrive at predefined distances during retraction of the interventional devices.

In some embodiments, maintaining at least some of the distal ends of the interventional devices within a predefined distance from each other can beneficially maintain a desired overall stack stiffness profile and/or one or more desired local stiffnesses or stiffness profiles at one or more positions or segments along the length of the concentric stack of interventional devices. This can provide better support for the stack of interventional devices and/or allow for easier translation and/or navigation of the stack along the vasculature of a patient. In some embodiments, the predefined distances may be desirable for performing a particular step of a procedure.

Predefined distances D1, D2, and/or D3 can be adjusted and/or includes different thresholds for different stages of the procedure. For example, in some embodiments, it may be desirable for the distal ends of a plurality of interventional devices to be arranged at particular positions relative to one another for a particular step of a surgical procedure. In some embodiments, it may be desirable that the distal ends of the plurality of interventional devices may be arranged at different particular positions relative to one another at a different step of the surgical procedure. For example, D2 may be set up to include a first value for a first stage (e.g., an access stage) and to include a second value that can be smaller and/or larger than the first value for a second stage (e.g., a procedure stage) of the procedure. In some embodiments, a plurality of interventional devices linked to a single control can be driven from a first set of positions to a second set of positions in which at least some of the interventional devices are at different relative positions to one another in comparison to the first set of positions (e.g., by moving the linked interventional devices different distances relative to one another and/or at different axial velocities relative to one another).

In some embodiments, the amount of movement and/or the value of the axial velocities of different interventional devices can change based on the extent of the relative distances between the interventional devices when driving is initiated while in the auto stack drive mode. For example, if two devices are positioned at a first relative distance from one another and the desired relative distance is smaller than the first relative distance, one of the devices may move at a greater relative velocity in comparison to the other one of the devices than if the initial relative distance was smaller. Similarly, the amount of movement and/or the values of the axial velocities may change based on the overall distance available for arriving at the desired configuration of devices. For example, if the auto stacking drive mode is initiated during retraction when two interventional devices are positioned at the M1 segment, they may adjust to the desired configuration over a longer period of time or longer overall distance than if the auto stacking drive mode was initiated during a retraction starting at the aortic arch.

In some embodiments, when two or more of the interventional devices 6220, 6240, 6260, and 6280 are driven together (e.g., in response to actuation of a single user input or control, such as a joystick) the velocities of the two or more interventional devices 6220, 6240, 6260, 6280 can be automatically adjusted (e.g., by a control system) to maintain or achieve desired separation distances. In some embodiments, the velocities of different interventional devices can be adjusted as percentage of a velocity of one of the interventional devices (e.g., of a leading interventional device). For example, in certain embodiments, one of the interventional devices may be driven at a first velocity that corresponds to an amount of actuation of a control, such as a joystick, and the other interventional devices may be driven at velocities that are a percentage of the first velocity. In some embodiments, the velocities of one or more of the interventional devices can be adjusted as a percentage of commanded velocity by the actuation of a control, such as a joystick. In some embodiments, the velocities of one or more interventional devices can be adjusted based on the distance over which the adjustment is performed. In some embodiments, the velocities of one or more interventional devices can be adjusted based as a percentage of a commanded velocity of one of the interventional devices and adjusted based on the distance over which the adjustment is performed.

In some embodiments, relative positions and relative velocities of different interventional devices can be changed for different procedure stages, for example, in response to a selection of a particular procedure stage using a user input of a controller. In some embodiments, relative positions and relative velocities of different interventional devices can be changed at different segments of a single procedure stage. In some embodiments, the ability to modify parameters (e.g., relative positions and relative velocities) at different procedure stages can be selectively enabled and disabled, for example, in response to an input from a user using a controller or user interface.

In some cases, the distal ends of the interventional devices 6220, 6240, 6260, 6280 are aligned when an interventional procedure starts. That is, there is no separation between the distal ends of the interventional devices. As one or more of the interventional devices are axially translated through the vasculature of the patient (e.g., in response to actuation of a single input by a user, such as a joystick), the interventional devices can be advanced such that the distal ends 6220a, 6240a, 6260a, 6280a create and/or maintain a distance between each other. In some embodiments, each of the interventional devices can be advanced at the same velocity to maintain a predefined separation and/or at different velocities to increase and/or decrease the predefined separation. For example, in cases where the distal end 6220a of the first interventional device 6220 is positioned distal to the distal ends of the other interventional devices, as shown in FIG. 24A, the second, third, and fourth interventional devices can be advanced at different velocities to increase, decrease, and/or maintain distances D1, D2, and/or D3 between their distal ends.

In some cases, the first interventional device 6220 may remain stationary while the second, third, and fourth interventional devices are translated over the first international device 6220 (e.g., to maintain the distal end 6220a of first interventional device 6220 at a desired distance from one or more of distal ends 6240a, 6260a, and 6280a or in response to an actuation of a user input, such as a joystick, that controls movement of only the second, third, and fourth interventional devices). As the second, third, and fourth interventional devices are translated over the first interventional device 6220, the distance D1 may decrease until the distal end 6240a is translated distal to the distal end 6220a, as shown in FIG. 24C. When the second, third, and fourth interventional devices start being translated over the first interventional device 6220, the second interventional device 6240 may be advanced at a higher velocity than the third and fourth interventional devices, as shown by FIGS. 24A and 24B. In some embodiments, once the distance D2 reaches a certain threshold, the second, third, and fourth interventional devices may be advanced at the same velocity to maintain the distances D2 and D3.

In some embodiments, the interventional devices can be positioned in a predefined configuration at the beginning of a procedure (e.g., when introduced at a patient access point). In some embodiments, when the auto stacking drive mode is utilized during retraction of two or more interventional devices, the two or more interventional devices can be driven as described herein to return to the initial configuration.

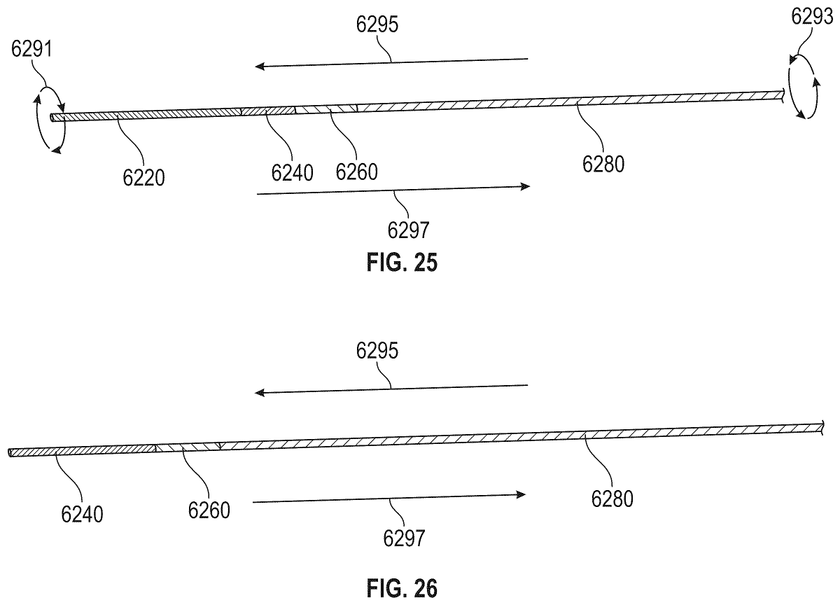

FIG. 25 illustrates another example of a drive mode for a control mechanism. The drive mode illustrated in FIG. 25 can be applied to and/or otherwise be incorporated into any of the control mechanisms described herein and the respective hubs and/or interventional devices. For example, the drive mode can be used to control axial and/or rotational translation of the first interventional device 6220, the second interventional device 6240, the third interventional device 6260, and/or the fourth interventional device 6280.

During a procedure, it may be desirable to automatically rotate interventional devices. In certain embodiments, one or more of the interventional devices can be automatically rotated as the interventional devices are axially translated. In some embodiments, one or more of the interventional devices can be automatically rotated whenever the one or more interventional devices are in a drive ready state (e.g., whenever the one or more interventional devices are linked to a control so that a user can axially translate the one or more interventional devices). In some embodiments, one or more interventional devices can be automatically rotated continuously in one direction (e.g., clockwise or counterclockwise). In some embodiments, the single direction can be selected by the user and may be maintained until the user selects a different direction.

In some embodiments, one or more interventional devices can be automatically rotated in an oscillatory manner, for example by rotating in a first direction (e.g., clockwise or counterclockwise) and the rotating in a second direction opposite of the first direction. In some embodiments, the one or more interventional devices can be oscillated in a sinusoidal pattern. In some embodiments, the one or more interventional devices can rotate in an oscillatory manner by rotating in each direction for a particular number of revolutions or period of time.

In some embodiments, one or more interventional devices can be automatically rotated at a constant velocity. In other embodiments, the rotational velocity can be ramped, for example, to gradually increase from zero to a predefined velocity. In some embodiments, the velocity can change depending on an axial velocity of the interventional device. For example, an interventional velocity may rotate at a higher velocity in response to a higher axial velocity than a lower axial velocity. In certain embodiments, an amount of rotation may correspond to an amount of axial movement. In some embodiments, velocity, direction, and/or extent of rotation can be modified through configuration parameters of the control system. The time, mode, and/or the stage of the procedure during which the rotation occurs can also be adjusted. For example, automatic rotation may occur when the interventional devices are being inserted, when the interventional devices are being retracted, when the interventional devices are stationary, and/or when the interventional devices are first introduced to the vasculature.

In some procedures, due to non-linear torque response of an interventional device in the anatomy of a patient, it can be difficult to immediately view rotation in a fluoroscopic image, which may create difficulty for a user to control rotation. Additionally, it may be difficult for a user to actively control both simultaneous rotation and axial movement of an interventional device, which may be desired in certain anatomies. By automatically rotating the interventional devices, users can focus on insertion and/or retraction of the interventional devices. Automatic rotation can also beneficially achieve a desired and/or more predictable torque response at a distal end of the interventional devices.

In some embodiments, a user can select between continuous rotation in one direction or oscillatory rotation. In some embodiments, a user can select whether a rotation will occur while an interventional device is in a drive ready state or only when the interventional device is actively being axially advanced or retracted.

In some embodiments, one or more of the interventional devices 6220, 6240, 6260, and/or 6280 can continuously rotate in a clockwise direction 6291 and/or a counterclockwise direction 6293. In some cases, the direction of rotation can be set to the clockwise direction 6291 and/or the counterclockwise direction 6293. The direction of rotation can depend on the last used direction of rotation. For example, if a user selects to rotate one or more of the interventional devices in the clockwise direction 6291 (e.g., using a control mechanism), then the one or more interventional devices will rotate in the clockwise direction 6291 until the user selects the counterclockwise direction 6293.

In certain embodiments, one or more of the interventional devices 6220, 6240, 6260, and/or 6280 can rotate in an oscillatory manner in a clockwise direction 6291 and a counterclockwise direction 6293. For example, the interventional devices may rotate in a sinusoidal pattern, rotating in one direction (e.g., the clockwise direction 6291) up to a predefined rotational amount, then change direction to the counterclockwise direction 6293 up to a predefined rotational amount, then change direction to the clockwise direction 6291 again.

During a procedure, it may be desirable to axially oscillate interventional devices. In certain embodiments, one or more of the interventional devices 6220, 6240, 6260, and/or 6280 can be automatically axially oscillated as the interventional devices are axially translated. In some embodiments, one or more of the interventional devices can be automatically axially oscillated whenever the one or more interventional devices are in a drive ready state (e.g., whenever the one or more interventional devices are linked to a control so that a user can axially translate the one or more interventional devices).

In certain embodiments, one or more of the interventional devices can be automatically axially oscillate by moving back and forth between a distal direction 6295 (e.g., an insertion direction) and a proximal direction 6297 (e.g., a retraction direction). In some embodiments, when a control mechanism is actuated to move one or more of the interventional devices axially, and the axial oscillation drive mode is active, the one or more interventional devices may translate distally and/or proximally in a sinusoidal pattern while advancing in the direction commanded by the control mechanism. For example, the interventional devices may translate in the distal direction 6295 up to a predefined axial distance, then translate in the proximal direction 6297 up to a predefined axial distance, then translate in the axial direction 6295 again. The predefined axial distance when the interventional devices translate in the distal direction 6295 may be different than the predefined axial distance when the interventional devices translate in the proximal direction 6297. This can beneficially allow the interventional devices to be inserted deeper into the vasculature and/or be retracted from the vasculature in spite of the axial oscillation. For example, if axial movement of an interventional device in the distal direction 6295 is commanded, the interventional device may automatically oscillate using a greater predefined distance in the distal direction 6295 than in the proximal direction 6297.

In some cases, one or more of the interventional devices 6220, 6240, 6260, and/or 6280 may oscillate in different directions from one another when the axial oscillation mode is active. For example, the second interventional device 6240 may translate in the distal direction 6295 while the third interventional device 6260 translates in the proximal direction 6297. When the second interventional device changes the direction of the translation (e.g., when it switches translation from the distal direction 6295 to the proximal direction 6297), the third interventional device 6260 may switch translation from the proximal direction 6297 to the distal direction 6295.

In the axial oscillation mode described herein, the amplitude and/or frequency of oscillation can be configurable. The time, mode, and/or the stage of the procedure during which the axial oscillation occurs can also be adjusted. For example, axial oscillation may occur when the interventional devices are being inserted, when the interventional devices are being retracted, when the interventional devices are stationary, and/or when the interventional devices are first introduced to the vasculature.

In some embodiments, automatic axial oscillation may be performed together with automatic rotation (e.g., continuous rotation in one direction or oscillatory rotation). The combination of automatic rotation and axial oscillation can beneficially allow users to cycle through many combinations of orientations and/or positions of the interventional devices, which can enable a systematic trial and error approach to enter a specific vessel within the vasculature of the patient. For example, in some embodiments, at each point throughout a single cycle of axial oscillation and automatic rotation, the axial position and rotational position can be different.

Automatic oscillation can be applied to all or just some of the interventional devices 6220, 6240, 6260, and/or 6280. For example, in some cases, an axial oscillation drive mode may be applied to all of or one or more trailing interventional devices but not a leading interventional device (e.g., the interventional device positioned further distally along the vasculature of a patient during distal advancement of one or more interventional devices). As shown in FIG. 26, the second interventional device 6240 can be the leading interventional device. When the control mechanism is actuated to translate the interventional devices 6240, 6260, and 6280 axially in the distal direction 6295, the third interventional device 6260 and the fourth interventional device 6280 may translate according to the axial oscillation mode (e.g., alternating translation between the proximal direction 6297 and the distal direction 6295) while the second interventional device 6260 translates in a non-oscillatory mode (e.g., in the distal direction 6297). In some embodiments, the number of trailing catheters that automatically oscillate can be changed, for example, based on the stage of a procedure (e.g., as selected by a user operating a control mechanism).

In some cases, the axial oscillation mode can be activated and/or deactivated for each of the interventional devices.

When translating the interventional devices through tortuous anatomy, the interventional devices may bend and contort. This may result in high forces being applied laterally to the interventional devices, which may push the interventional devices against each other. The high forces may lead to high friction, which may impede user-controlled motion of the interventional devices, and cause tension buildup. Tension buildup may result in lack of response for device translation, and/or sudden jumps in device position due to release of the tension. The lateral forces may also result in buildup of tension when the interventional devices are pressed against vessel walls. The friction between the interventional devices and the anatomy may cause a lack of response during high tension stages, and/or sudden jumps in device position when tension is released. Automatic axial oscillation of the interventional devices relative to each other and/or relative to the vessel walls, may prevents the buildup of tension as the relative movement of the interventional devices allows the conveyance of lateral force down the interventional devices, thereby preventing buildup of tension.

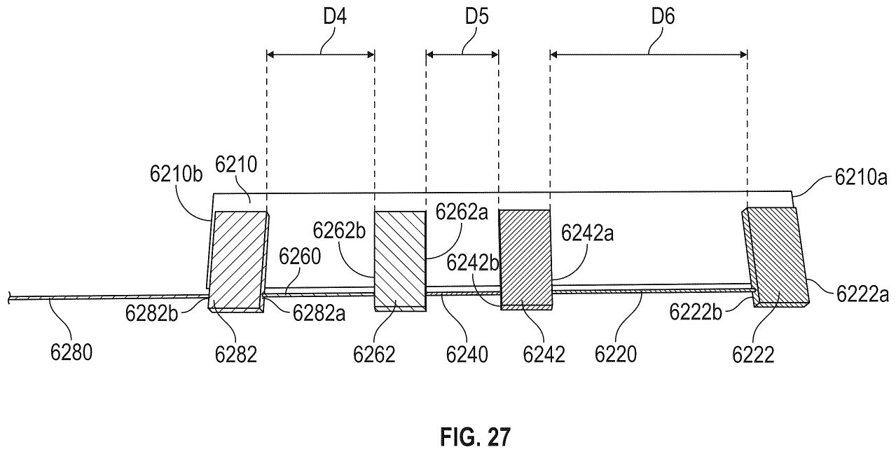

In some embodiments, two or more interventional devices may be automatically linked to each other (e.g., so that translation of one of the interventional devices will cause translation of the other interventional device) based on the position of their corresponding hub assemblies or hup adapters and/or based on the position of the distal ends of the interventional devices. As previously described herein, each interventional device may be coupled to a corresponding hub assembly. For example, the first interventional device 6220 can be coupled to a first hub assembly 6222, the second interventional device 6240 can be coupled to a second hub assembly 6242, the third interventional device 6260 can be coupled to a third hub assembly 6262, and the fourth interventional device 6280 can be coupled to a fourth hub assembly 6282, as shown in FIG. 27. Axial translation of the hub assemblies along a support table 6210 may cause axial translation of the corresponding interventional devices.

Each of the hub assemblies may be separated from each other. For example, a proximal end 6282a of the fourth hub assembly 6282 and a distal end 6262b of the third hub assembly 6262 may be separated by a distance D4. A proximal end 6262a of the third hub assembly 6262 and a distal end 6242b of the second hub assembly 6242 may be separated by a distance D5. A proximal end 6242a of the second hub assembly 6242 and a distal end 6222b of the first hub assembly 6222 may be separated by a distance D6. Two or more of the interventional devices 6220, 6240, 6260, and 6280 may be linked to each other based on the distance between their corresponding hub assemblies 6222, 6242, 6262, and 6282. For example, if the distance D4 increases beyond a threshold value (e.g., 1 cm, 5 cm, 10 cm, 15 cm, 20 cm, 30 cm, 50 cm, etc.) due to the fourth hub assembly 6282 moving away from the third hub assembly 6262, then the translation commanded by the control mechanism to the fourth hub assembly 6262 can be mirrored to the third hub assembly 6282 (e.g., resulting in both interventional devices being translated). In some such cases, decreasing the distance D4 below the threshold value (e.g., translating the third hub assembly 6262 closer to the fourth hub assembly 6282) may disable linking of the fourth hub assembly 6282 to the third hub assembly 6262.

In some procedures, two or more interventional devices, hub assemblies, and/or hub adapters may be linked when controlled movements of one or more of the interventional devices, hub assemblies, and/or hub adapters would result in relative positions between the interventional devices, hub assemblies, and/or hub adapters at distances greater than a total available separation distance (e.g., due to the length of the drive surface of the drive table).

In some embodiments, linked interventional devices, hub assemblies, and/or hub adapters can include a primary device, hub assembly, and/or hub adapter and one or more secondary devices, hub assemblies, and/or hub adapters. Movement of the primary device, hub assembly, and/or hub adapter can cause movement of the secondary device, hub assembly, and/or hub adapter in the same direction by the same magnitude and/or velocity. Movement of a control to move the secondary device, hub assembly, and/or hub adapter, but not the primary device, hub assembly, and/or hub adapter may not cause the primary device to move. In some embodiments, movement of a control to move the secondary device, hub assembly, and/or hub adapter, but not the primary device, hub assembly and/or hub adapter, in a direction that would increase a separation distance between the primary device, hub assembly, and/or hub adapter and the secondary device, hub assembly, and/or hub adapter may result in no movement of either the primary device, hub assembly, and/or hub adapter or the secondary device, hub assembly, and/or hub adapter. In some embodiments, movement of a control to move the secondary device, hub assembly, and/or hub adapter, but not the primary device, hub assembly, and/or hub adapter, in a direction that would decrease the separation distance between the primary device, hub assembly, and/or hub adapter and the secondary device, hub assembly, and/or hub adapter may cause the secondary device to move without movement of the primary device, hub assembly, and/or hub adapter and/or may unlink the secondary device, hub assembly, and/or hub adapter from the primary device, hub assembly, and/or hub adapter.

In some procedures, two or more interventional devices, hub assemblies, and/or hub adapters may be linked to each other when an instructed relative movement between the devices would exceed a total distance available for movement of the interventional devices along the drive table. For example, in certain embodiments, the drive table 6210 may move axially to provide for additional axial movement of the interventional devices coupled to the drive table. In such embodiments, movement of interventional devices, hub assemblies, and/or hub adapters, may be linked together. For example, if the distal most hub assembly 6282 is moved to its distal most position along the drive table 6210, but further distal advancement of its corresponding interventional device 6280 is instructed, the drive table 6210 may be moved distally. In such embodiments, one or more of the other interventional devices that are not instructed to move may automatically move in the opposite direction (e.g., proximally) so as to retain their relative positions with respect to the distal most hub assembly 6282 and/or a patient access point. In some embodiments, movement of the distal most hub assembly 6282 may be instructed that would result in a relative distance between the distal most hub assembly 6282 and the proximal most hub assembly 6222 greater than the available length of the drive table 6210. In such cases, movement of the proximal most hub assembly 6222 may be linked to the movement of the distal most hub assembly 6282. In other words, while adjusting in response to movement of the drive table, the proximal hub assembly 6222 may reach a proximal most position along the drive table and be prevented from further movement while the drive table continues to move in the distal direction. In response, the drive system may compensate by temporarily linking the movement of the proximal most hub assembly 6222 with the movement of the distal most hub assembly 6282 so that the proximal most hub assembly 6222 moves in unison with the distal most hub assembly 6282. For example, when linked, the proximal most hub assembly 6222 and distal most hub assembly 6282 may move in a proximal or distal direction by the same magnitude and/or at the same speed. In some embodiments, while the drive table is advancing, the proximal most hub assembly 6222 and the distal most hub assembly 6282 may move proximally or distally by the same magnitude and/or same speed by maintaining their positions on the drive table while the drive table advances (and while other hub assemblies move proximally and distally to compensate for movement of the drive table 6210.

In some embodiments, the proximal most hub assembly 6222 can be unlinked from the distal most hub assembly in response to a user manipulation using the controller. In certain embodiments, while proximal most hub assembly 6222 and distal most hub assembly 6282 are linked, the drive system can track the desired relative position of the proximal most hub assembly 6222 relative to the distal most hub assembly 6282 and/or relative to a reference point and adjust the proximal most hub assembly 6222 to the desired position once sufficient space is available along the drive table 6210.

In some procedures, it may be desirable to prevent a distal end of one or more of the interventional devices from separating from the distal end of another interventional device beyond a threshold value. For example, it may be desirable to maintain a distance between the distal end 6220a of the first interventional device 6220 (e.g., a guidewire) and the distal end 6280a of the fourth interventional device 6280 maintained below a threshold value. When translating the fourth interventional device 6280 away from the first interventional device such that the distance between the distal ends 6280a and 6220a reaches the threshold value, the first interventional device 6220 and the fourth interventional device may be linked to prevent the distance between the distal ends 6280a and 6220a from exceeding the threshold value. In such cases, decreasing the distance between the distal ends 6280a and 6220a below the threshold value (e.g., translating the distal ends 6280a and 6220a closer to each other) may disable linking of the first interventional device 6220 and the fourth interventional device 6280.

In some cases, the interventional devices may be linked to prevent the distal ends of the interventional devices from separating beyond a threshold distance from the distal end of a leading device. For example, when the second interventional device 6240 is the leading device and is being translated in a distal direction, the third and fourth interventional devices 6260, 6280 may be linked to the second interventional device 6240 if the distances between the distal ends 6260a, 6280a and the distal end 6240a reach predefined values. When linked, translation of the leading device (e.g., the second interventional device 6240) will cause translation of the third and fourth interventional devices 6260, 6280. Automatic linking may beneficially allow users to focus on control of the leading device without the risk of the trailing devices falling to far behind, which may negatively affect the stiffness of the interventional device stack at certain positions. In some embodiments, different devices may be automatically linked at different portions of a procedure or in different drive modes. For example, during an access stage as described herein, all of the interventional devices 6220 (e.g., a guidewire), 6240 (e.g., an access catheter), 6260 (e.g., a procedure catheter), and 6280 (e.g., a guide catheter) may be configured to link to a leading interventional device if a separation distance reaches a predefined value. In some embodiments, for example, if driving the interventional devices 6240, 6260, and 6280 using a single control of a controller in an access drive mode as described herein, the interventional device 6220 can be configured to link to the leading one of the interventional devices 6240, 6260, and 6280 if a separation distance reaches a predefined value. In some embodiments, different devices may be configured to link in a procedure stage or when using a procedure drive mode.

In some embodiments, when different controls (e.g., joysticks) are used to retract and/or advance different interventional devices simultaneously, the velocities at which the interventional devices are retracted and/or advanced can be matched. For example, when retracting the second interventional device 6240, which can be the distal-most device in the vasculature, using a first input on a control mechanism and retracting the third interventional device 6260 using a second input on the control mechanism, the velocity at which the second and third interventional devices are retracted may be automatically matched even if the velocity commanded by the first and second inputs are different. In some cases, the retraction and/or insertion velocity of two or more devices will be matched when both the first and second inputs are actuated to reach a predefined percentage of a maximum retraction magnitude threshold (e.g., the joysticks of the control mechanism are pushed to their full extent). Matching velocities during retraction and/or insertion can beneficially ensure that all interventional devices retract and/or advance uniformly and that the interventional devices do not lag behind each other.

In some embodiments, the distances between adjacent hub assemblies 6222, 6242, 6262, and 6282 and/or their corresponding hub adapters may be monitored to determine the possibility of a collision and prevent collisions from occurring. In some embodiments, if the distance between two hub assemblies (e.g., distance D4, D5, or D6) and/or two hub adapters is above a predefined threshold, collision between the hub assemblies and/or hub adapters may not be a concern, and the hub assemblies and/or hub adapters are allowed to move closer to each other. If the distance between two hub assemblies (e.g., distance D4, D5, or D6) and/or two hub adapters is below a first predefined threshold but above a second predefined threshold, movement of the hub assemblies and/or hub adapters closer to one another may be prevented. The hub assemblies and/or hub adapters may be allowed to move but only to increase the distance between hub assemblies and/or hub adapters. If the distance between two hub assemblies (e.g., distance D4, D5, or D6) and/or two hub adapters is below the second threshold, the hub assemblies and/or hub adapters may be prevented from any movement. This can beneficially prevent one hub assembly from colliding with another hub assembly and/or one hub adapter from colliding with another hub adapter, which may damage the hub assemblies, hub adapters, and/or the corresponding interventional devices and/or injure the patient.

In some embodiments, the control system can include first and/or second thresholds based on desired distances between sections of interventional devices in addition to and/or alternatively to distances between adjacent interventional devices and/or hub assemblies. In some embodiments, the first and second thresholds can be adjusted depending on the interventional devices being used. For example, some interventional devices may be advanced farther into another interventional device than others. Different interventional devices may have different regions that are incompatible with one another. For example, an outer device may have a tapered region that is too small for a portion of a device extending therethrough. In such cases, translation of a tapering catheter within another catheter may be limited to prevent the tapering catheter from getting stuck inside the other catheter.

In some embodiments, a maximum separation distance between adjacent hub assemblies and/or hub adapters may be limited to prevent buckling of the interventional devices associated with the hub assemblies and/or hub adapters. In some embodiments, a maximum separation distance between adjacent hub assemblies may be limited to prevent a separation distance that exceeds a length of an anti-buckling system or device extending between hub assemblies. In some embodiments, if the distance between two hub assemblies (e.g., distance D4, D5, or D6) and/or two hub adapters is below a predefined threshold, the separation between adjacent hub assemblies may not be a concern and the hub assemblies may be permitted to move away from each other. If the distance between two hub assemblies (e.g., distance D4, D5, or D6) and/or two hub adapters is above the first predefined threshold but below a second predefined threshold, movement of the hub assemblies and/or hub adapters further from one another may be prevented. The hub assemblies and/or hub adapters may be allowed to move to decrease the distance between the hub assemblies and/or hub adapters. If the distance between two hub assemblies (e.g., distance D4, D5, or D6) and/or two hub adapters is above the second threshold, the hub assemblies and/or hub adapters may be prevented from any movement. The first and second thresholds can be adjusted.

In some cases, the one or more of the hub assemblies 6222, 6242, 6262, and 6282 and/or their corresponding hub adapters may be prevented from translating if a distance between the hub assemblies and/or hub adapters and a proximal end or a distal end of the support table 6210 is below a threshold level. For example, if a distance between the distal end 6282b of the fourth hub assembly 6282 and a distal end 6210b of the support table 6210 is below a threshold value, translation of the fourth hub assembly 6282 in the direction of the distal end 6210b can be prevented. Alternatively, in some embodiments, the support table 6210 may be moved distally by an amount configured to maintain the distance between the distal end 6282b of the fourth hub assembly 6282 and the distal end 6210b of the support table 6210 above the threshold value. Similarly, if a distance between the proximal end 6222a of the first hub assembly 6222 and a proximal end 6210a of the support table 6210 is below a threshold value, translation of the first hub assembly 6222 in the direction of the proximal end 6210a can be prevented. Alternatively, in some embodiments, the support table 6210 may be moved proximally by an amount configured to maintain the distance between proximal end 6222a of the first hub assembly 6222 and the proximal end 6210a of the support table 6210 above the threshold value.

In cases where not all of the hub assemblies 6222, 6242, 6262, and 6282, have an interventional device installed to them, the anti-collision modes described herein may not apply to the hub(s) without an installed interventional device or may have different threshold values or anti-collision functions. For example, if the fourth interventional device 6280 is not installed in the fourth hub assembly 6282 (e.g., such only three interventional devices are installed), translation of the third hub assembly 6262 in the direction of the fourth hub assembly 6282 may cause the fourth hub assembly 6282 to translate if the distance D4 is below a predefined threshold. This can beneficially increase the range of motion of hub assemblies with an installed interventional device.

In some embodiments, one or more of the drive modes described herein can be performed simultaneously. In some embodiments, one or more automatically performed functions of the drive modes described herein may be controlled by a control system.

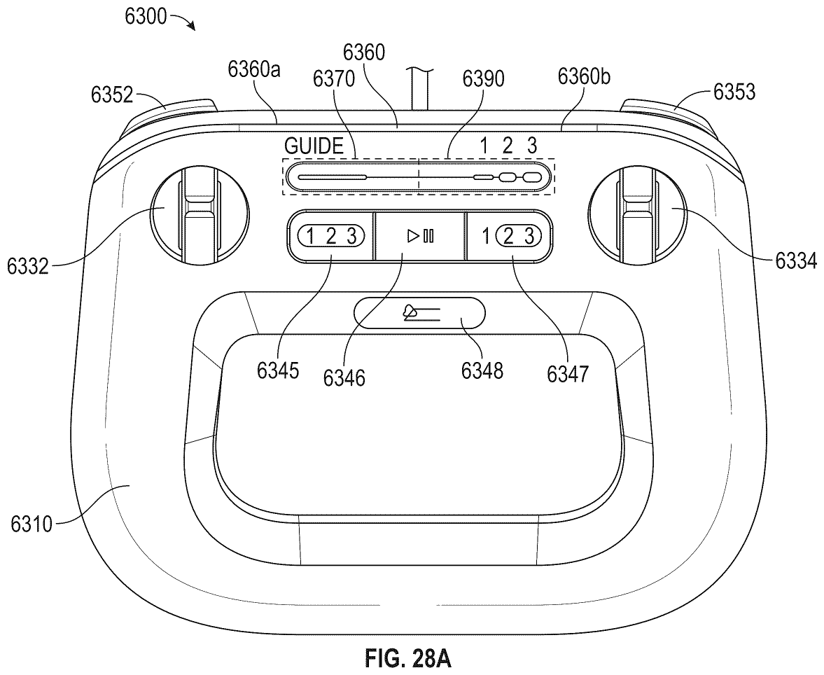

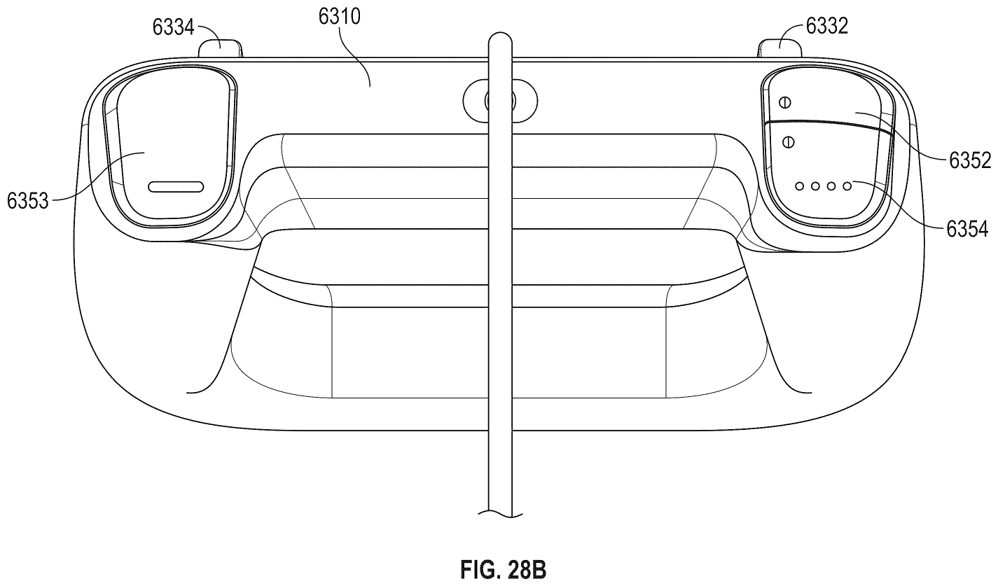

FIGS. 28A-28B illustrate another embodiment of a control mechanism 6300 for manipulating interventional devices driven by (or otherwise associated with) respective hub assemblies. As shown in FIGS. 28A-28B, the control mechanism 6300 can be in the form of a handheld controller 6310. The control mechanism may include any of the same or similar features and/or functions as any of the other control mechanisms described herein, such as the control mechanism 5300.

The control mechanism 6300 can include a handheld controller 6310 having one or more controls (e.g., inputs), which may be in the form of buttons and/or joysticks or any other suitable actuators. As shown, the control mechanism 6300 can include a first control or first joystick 6332. The control mechanism 6300 can include a second control or second joystick 6334. The control mechanism 6300 can include a plurality of buttons 6345, 6346, 6347, 6348, and a plurality of shoulder or bumper buttons 6352, 6353, 6354. In some embodiments, the control mechanism 6300 can include a light bar 6360. In some embodiments, the control mechanism 6300 can include a first indicator 6370 and a second indicator 6390.

The shoulder button 6352 can be pressed and/or held to inject fluids, such as contrast or saline, via one or more of the interventional devices. The bumper button 6354 can be pressed to provide a small increment of a fluid (such as contrast and/or saline) puff (e.g., about 1 mL per puff). The button 6346 can be pressed to activate and deactivate control of the hub assemblies and/or interventional devices. For example, pressing the button 6346 can pause or stop movement of the hub assemblies and/or interventional devices and pause or stop control of the hub assemblies and/or the interventional devices, such that the hub assemblies and/or the interventional devices will not move in response to actuation of the controls in the control mechanism 6300. To resume controlling the hub assemblies and/or interventional devices, the button 6346 can be pressed again.

Each of the first joystick 6332 and the second joystick 6334 may correspond to and drive movement of one or more hub assemblies and/or interventional devices.

In some embodiments, the first joystick 6332 can be linked to a first particular hub assembly and/or interventional device, such as a hub assembly associated with a guidewire. For example, with respect to the drive system shown in FIG. 27, which includes four hub assemblies and interventional devices, the first joystick 6332 may be linked to a hub assembly 6222 and/or its corresponding interventional device 6220 (e.g., a guidewire). The second joystick 6334 can be selectively linked to one or more of the remaining hub assemblies 6242, 6262, 6282 and/or interventional devices 6240 (e.g., an access catheter), 6260 (e.g., a procedure catheter), 6280 (e.g., a guide catheter), for example, by a user making a selection on the controller 6310 (e.g., by pressing one or more buttons). For example, pressing the button 6345 may cause the second joystick 6334 to be linked to all of the remaining hub assemblies 6242, 6262, 6282 and/or interventional devices 6240, 6260, 6280. Actuation of the second joystick 6334 when the second joystick 6334 is linked to three hub assemblies and/or interventional devices will cause all of the linked hub assemblies and interventional devices to translate as commanded by the second joystick 6334. Movement of an access catheter, procedure catheter, and guide catheter together may be desirable during a first stage or access stage of a procedure, as described herein.

In some embodiments, pressing the button 6446 may cause the second joystick 6334 to be linked with at least two hub assemblies and/or at least two interventional devices such as hub assemblies 6262 and 6282 and/or interventional devices 6260 (e.g., a procedure catheter) and 6280 (e.g., a guide catheter). Movement of a procedure catheter and guide catheter together may be desirable during a second stage or procedure stage of a procedure as described herein.

In some embodiments, it may be desirable to move each of the hub assemblies 6242, 6262, 6282 and/or interventional devices 6240, 6260, 6280 individually during certain portions of a procedure as described herein. Pressing the bumper button 6353 can beneficially allow users to toggle between interventional devices. For example, if the second joystick is linked to a second hub assembly and/or interventional device, pressing the bumper button 6353 once may link the second joystick 6334 to a third hub assembly and/or interventional device. Pressing the bumper button 6353 a second time may link the second joystick 6334 to a fourth hub assembly and/or interventional device. Pressing the bumper button 6353 a third time may link the second joystick 6334 to the second hub assembly and/or interventional device again. Thus, users can easily toggle between hub assemblies and/or interventional devices by pressing the bumper button 6353 as needed.

To indicate which hub assemblies and/or interventional devices are linked to the second joystick 6334, the second indicator 6390 can provide a visual indication about the hub assemblies and/or the interventional devices linked to the second joystick 6334. For example, as shown in FIG. 28A, the second indicator 6390 can provide a visual indication in the form of a light corresponding to a hub assembly and/or interventional device linked to the second joystick 6334. The second indicator 6390 can beneficially allow users to easily identify which hub assemblies and/or interventional devices are linked to the second joystick 6334. In some cases, the second indicator 6390 may display a light for each hub assembly and/or interventional device linked to the second joystick 6334.

In some embodiments, the first indicator 6370 can provide a visual indication regarding the status of the hub and/or interventional device associated with the first joystick 6332. For example, the first indicator 6370 may light up when the hub and/or interventional device associated with the first joystick 6332 is linked to the first joystick 6332.

The light bar 6360 can provide a visual indication about the status of the hub assemblies and/or the interventional devices. In some embodiments, the light bar 6360 can provide an indicate that one or more hub assemblies and/or interventional devices can be driven by the control mechanism 6300 or if control and/or movement of the one or more hub assemblies and/or interventional devices has been paused or stopped, for example, by pressing the button 6346. In some embodiments, lights extending at least partially around each joystick 6332 and 6334 may indicate that one or more hub assemblies and/or interventional devices are able to be driven by those joysticks.

In other embodiments, for example, a first portion 6360a of the light bar 6360 can provide a visual indication about the status of the fluidics system associated with the hubs and/or interventional devices. The first portion 6360a may display a first color to indicate a first status of the fluidics system (e.g., the fluidics system is ready and/or available) and a second color different than the first color to indicate a second status of the fluidics system (e.g., actively being used, not available, etc.). A second portion 6360b of the light bar 6360 can provide a visual indication about the drive modes associated with the hubs and/or interventional devices. For example, the second portion 6360b may display a first color to indicate that the control mechanism 6300 is in a specific drive mode (e.g., two hubs/devices are linked to the second joystick 6334, three hubs/devices are linked to the second joystick 6334, etc.) and a second color different than the first color to indicate a status of the hubs/devices (e.g., actively being driven, not available, etc.).

In some cases, the button 6348 may be used to start and/or stop aspiration via one or more of the interventional devices. For example, pressing the button 6348 once may start aspiration. Pressing the button 6348 again may stop aspiration. In some cases, aspiration can be applied only when the button 6348 is actively being pressed.

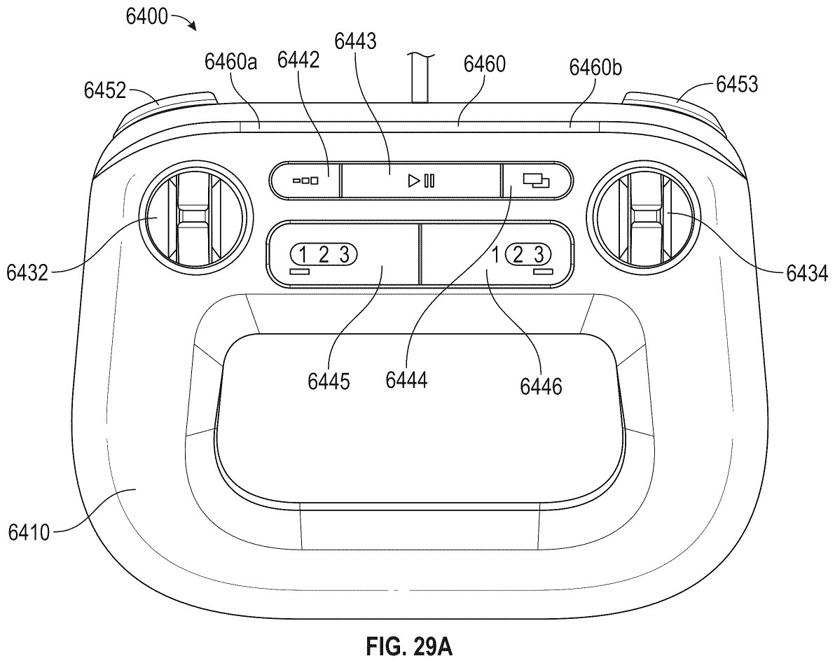

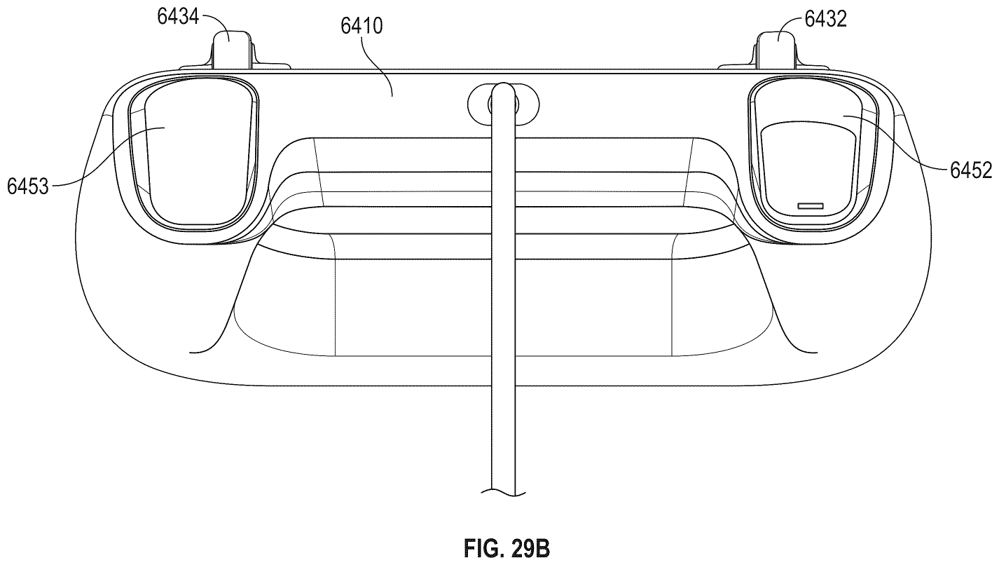

FIGS. 29A-29B illustrate another example control mechanism 6400 for manipulating interventional devices driven by (or otherwise associated with) respective hub assemblies. As shown in FIGS. 29A-29B, the control mechanism 6400 can be in the form of a handheld controller. The control mechanism may include any of the same or similar features and/or functions as any of the other control mechanisms described herein, such as the control mechanism 5300 and/or the control mechanism 6300.

The control mechanism 6400 can include a handheld controller 6410 having one or more controls, which may be in the form of buttons and/or joysticks or any other suitable actuators. The control mechanism 6400 can include a first control or first joystick 6432. The control mechanism 6400 can include a second control or second joystick 6434. The control mechanism 6400 can include a plurality of buttons 6442, 6443, 6444, 6445, 6446, and a plurality of shoulder or bumper buttons 6452, 6453. In some cases, pressing the button 6444 may toggle between two or more fluoroscopic views displayed on a user interface (e.g., the user interface 5400 and/or 6500). The control mechanism 6400 can also include a light bar 6460 having a first portion 6460a and a second portion 6460b. The bumper button 6453 can be used to toggle between hub assemblies and/or interventional devices as described in relation to the bumper button 6353 of FIGS. 28A and 28B.

In some embodiments, pressing the button 6445 may link the second joystick 6434 to three hub assemblies and/or interventional devices as described in relation to button 6345 of FIG. 28A. Pressing the button 6446 may link the second joystick 6434 to two hub assemblies and/or interventional devices as described in relation to button 6347 of FIG. 28A.

In some cases, pressing the 6442 may activate one or more of the drive modes described herein. For example, pressing the button 6442 once can activate an auto stacking drive mode described in relation to FIGS. 24A-24C. Pressing the button 6442 again may deactivate the auto stacking drive mode. Although reference is made to pressing the button 6442 to activate the auto stacking dive mode, the button 6442 can be configured to activate/deactivate any of the drive modes disclosed herein and/or to toggle between drive modes.

As shown in FIG. 29B, in certain embodiments, the control mechanism 6400 may not include separate controls for a fluid injection and puff, but may instead include one fluid control button 6452 that can be used to deliver fluid as it is depressed. For example, a short press may deliver a puff or burst while a long press may deliver a longer injection. In some embodiments, as shown in FIG. 29B, the button 6452 can have a surface split with distinct surfaces that can be pressed for separate injection and puff functions as described herein.

While particular combinations of controls (e.g., buttons, joysticks, etc.) and indicators are described above with respect to the controllers 6310 and 6410, different arrangements and combinations of controls and/or indicators may used to perform the functions described above.

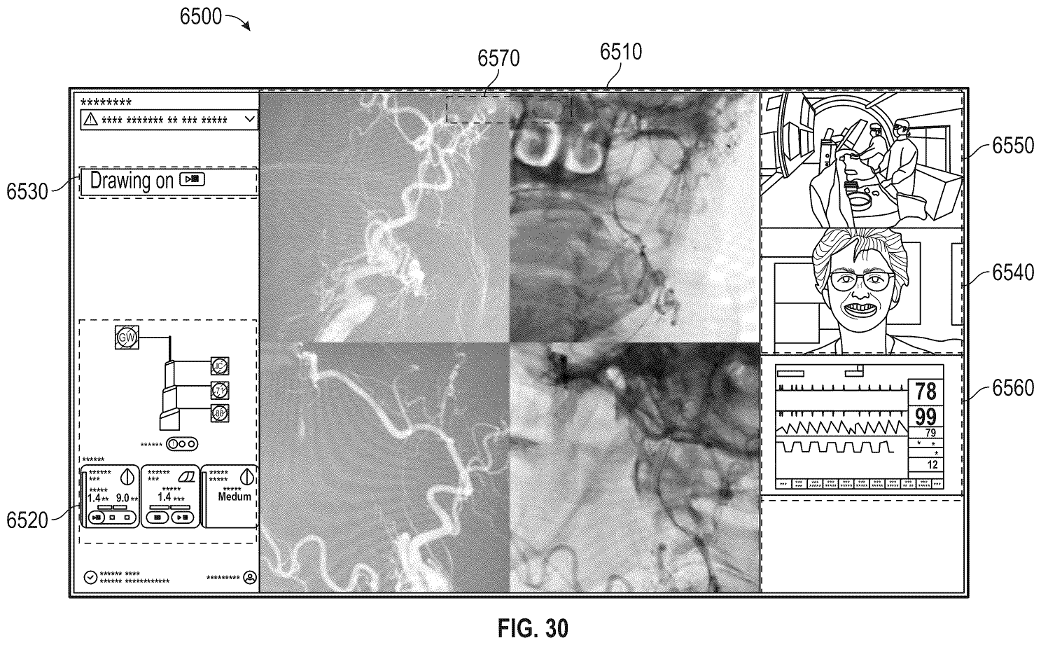

FIG. 30 is another embodiment of a user interface 6500 for facilitating control of the control mechanisms for interventional devices described herein. The user interface 6500 can include any of the same or similar features and/or functions as the user interface 5400.

The user interface 6500 can include a plurality of windows. For instance, the user interface 6500 can include an imaging window 6510, an instrument window 6520, a notification window 6530, a first video feed window 6540, a second video feed window 6550, a patient vitals window 6560, and/or a toggle indicator window or status indicator window 6570. In some cases, only some of the windows shown in FIG. 30 are displayed. In some cases, the user interface 6500 can be displayed on a capacitive screen, a resistive screen, a touch pad, or other touch-based sensing device. This can beneficially allow users to interact with the visual elements displayed on the user interface 6500. For example, in some embodiments, in addition to the user interface 6500 being displayed, users can adjust each of a plurality of parameters (e.g., velocity, rotational direction, axial position) displayed on the user interface 6500 by interacting with the user interface 6500. The plurality of parameters can be displayed on the instrument window 6520. Users can interact with the user interface 6500 by, for example, using basic gestures such as tapping, swiping, and/or dragging the components displayed on the capacitive screen, resistive screen, touch pad, or touch-based sensing device. Additionally or alternatively, the parameters displayed on the user interface 6500 can be adjusted by manipulating one or more physical buttons/components positioned adjacent the display in which the user interface 6500 is displayed.

The patient vitals window 6560 can display a patient's vital signs (e.g., body temperature, pulse rate, respiration rate, blood pressure, blood oxygen, etc.). In some cases, at least one window of the plurality of windows can include user profiles and/or settings. In some cases, instructions on how to move and/or operate the interventional devices associated with the user interface 6500 can be shown on the user interface 6500. The notification window 6530 can beneficially indicate whether a control mechanism, sch as the control mechanism 5300, 6300, 6400, is active.

In some cases, the status indicator window 6570 can indicate which hub assemblies and/or interventional devices are available to be linked to a control, such as a joystick 6334 or 6434, which hub assemblies and/or interventional devices are currently linked to the control. For example, as shown in FIG. 30, the status indicator window 6570 can indicate that three interventional devices are available to be linked with a control, such as joystick 6334 or 6434, and that all three interventional devices are currently linked to the control.

As previously described, users can toggle between individual hub assemblies and/or interventional devices controlled by a control, such as a joystick 6334 or 6434, by pressing a button on the control mechanism, such as button 6353 or 6453. When toggling between hub assemblies and/or interventional devices, it may be critical to assess which of the hub assemblies and/or interventional devices is currently linked to the control. As shown in FIGS. 31A-31C, the status indicator window 6570 can provide a visual indication to identify which hub assemblies and/or interventional devices are currently linked. For example, the status indicator window 6570 may include icons 6570a, 6570b, 6570c, each corresponding to one of the hub assemblies and/or interventional devices available to be linked to a control, such as a joystick 6334 or 6434. The first icon 6570a may correspond to a hub assembly, such as an insert catheter hub assembly, and/or interventional device, such as an insert catheter. The second icon 6570b may correspond to a hub assembly, such as a procedure catheter hub assembly, and/or interventional device, such as a procedure catheter. The third icon 6570c may correspond to a hub assembly, such as a guide catheter hub assembly, and/or interventional device, such as a guide catheter. To indicate which hub assemblies and/or interventional devices are linked to the control, the status indicator window 6570 may display the icon corresponding to any linked hub assembly and/or interventional device in a first color, texture, or pattern, and display the icon corresponding to any unlinked hub assembly and/or interventional device in a second color, texture, or pattern. As way of example, the first icon 6570a in FIG. 31A has a different appearance (e.g., green and opaque) than the appearance (e.g., gray and partially transparent) of the second and third icons 6570b, 6570c to indicate that the hub assembly and/or interventional device associated with the first icon 6570a is linked to the control.

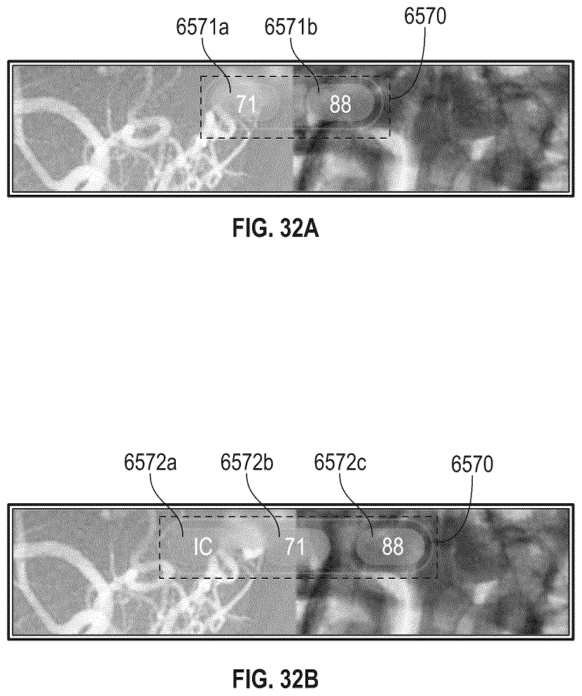

In some embodiments, the configuration of status indicator window 6570 as shown in FIGS. 31A-31B may be the configuration when only one of the hub assemblies and/or interventional devices is linked with the control. In some embodiments, the status indicator window 6570 can have a different configuration for providing a visual indication regarding that the control is linked to more than one hub assembly and/or interventional device. For example, as shown in FIG. 32A, the status indicator window 6570 can display two icons 6571a, 6571b. The icons 6571a, 6571b can have the same color, texture, or pattern, to indicate that both of the hub assemblies and/or interventional devices associated with the icons 6571a, 6571b are linked. As shown in FIG. 32B, the status indicator window 6570 can display three icons 6572a, 6572b, 6572c. The icons 6572a, 6572b, 6572c can have the same color, texture, or pattern, to indicate that all of the hub assemblies and/or interventional devices associated with the icons 6572a, 6572b, 6572c are linked to the control. As described herein, in some embodiments, multiple hub assemblies and/or interventional devices can be linked with a control during different stages or drive modes. For example, as described herein, in some embodiments, an access catheter, procedure catheter, and guide catheter may be linked to a single control, such as a joystick, during an access stage. This configuration may be referred to as an access drive mode. In some embodiments, a procedure catheter and guide catheter may be linked to a single control, such as a joystick, during a procedure stage. This configuration may be referred to as a procedure drive mode. In some embodiments, the configuration of the window 6570 in FIG. 32A may correspond to the procedure drive mode (e.g., when the procedure drive mode is actuated via an input, such as button 6347 or button 6446). In some embodiments, the configuration of the window 6570 in FIG. 32B may correspond to the access drive mode (e.g., when the access drive mode is actuated via an input, such as button 6345 or button 6445).

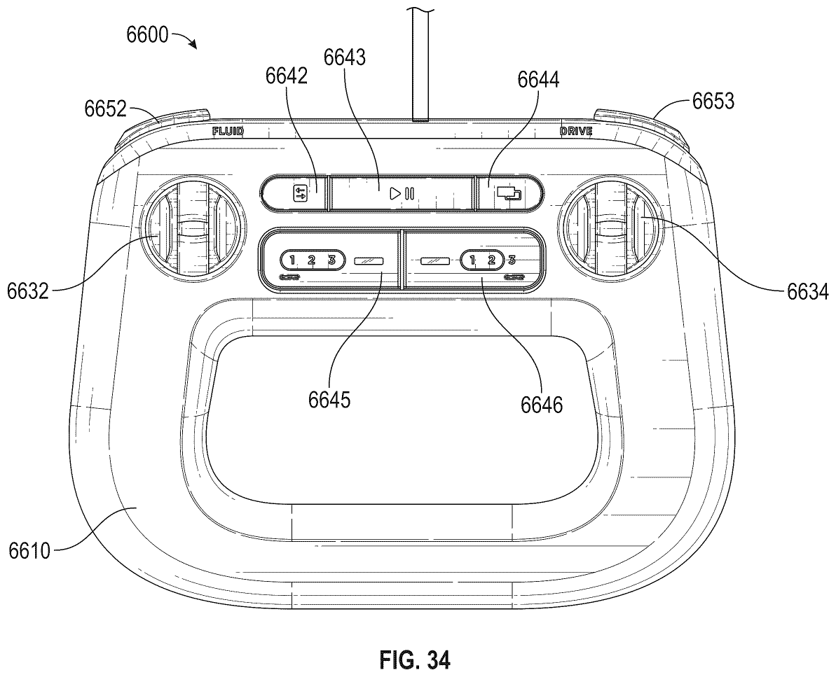

FIG. 34 illustrates another example control mechanism 6600 for manipulating interventional devices driven by (or otherwise associated with) respective hub assemblies. As shown in FIG. 34, the control mechanism 6600 can be in the form of a handheld controller. The control mechanism may include any of the same or similar features and/or functions as any of the other control mechanisms described herein, such as the control mechanism 5300, the control mechanism 6300, and/or the control mechanism 6400.

The control mechanism 6600 can include a handheld controller 6610 having one or more controls, which may be in the form of buttons and/or joysticks or any other suitable actuators. The control mechanism 6600 can include a first control or first joystick 6632. The control mechanism 6600 can include a second control or second joystick 6634. The control mechanism 6600 can include a plurality of buttons 6642, 6643, 6644, 6645, 6646, and a plurality of shoulder or bumper buttons 6652, 6653. In some embodiments, pressing the button 6643 may alternate between an active state and a paused state. In some embodiments, pressing the button 6643 while in the active state may pause movement of interventional devices and/or disable control of the interventional devices using the controller 6600. In some embodiments, pressing the button 6643 while in a paused state may enable control of the interventional devices using the controller 6600 so that the devices move in response to actuations of the controls of the controller. In some cases, pressing the button 6644 may toggle between reference views (e.g., between two or more fluoroscopic views) displayed on a user interface (e.g., the user interface 5400 and/or 6500). The bumper button 6653 can be used to toggle between hub assemblies and/or interventional devices as described in relation to the bumper button 6353 of FIGS. 28A and 28B and/or the bumper button 6453 of FIGS. 29A and 29B. The bumper button 6652 can be pressed and/or held to inject fluids, such as contrast or saline, via one or more of the interventional devices (e.g., an 1 innermost interventional device). The bumper button 6354 can be pressed to provide a small increment of a fluid (such as contrast and/or saline) puff (e.g., about 1 mL per puff). The bumper button 6652 can be held to provide larger amounts of fluid (e.g., contrast or saline).

In some embodiments, pressing the button 6645 may link the second joystick 6634 to three hub assemblies and/or interventional devices as described in relation to button 6345 of FIG. 28A and/or button 6445 of FIG. 29A. Pressing the button 6646 may link the second joystick 6634 to two hub assemblies and/or interventional devices as described in relation to button 6347 of FIG. 28A and/or button 6446 of FIG. 29A.

In some embodiments, the button 6642 can be activated to transition between a instrument window 6720 on the user interface and a drive table window 6820. Pressing the button once may cause the user interface to transition from displaying the instrument window 6720 to displaying the drive table window 6820 or transition from displaying the drive table window 6820 to displaying the instrument window 6720.

In some cases, the controller can include a button for activating one or more of the drive modes described herein. For example, pressing the button once can activate an auto stacking drive mode described in relation to FIGS. 24A-24C. Pressing the button again may deactivate the auto stacking drive mode. Although reference is made to pressing the button to activate the auto stacking dive mode, the button can be configured to activate/deactivate any of the drive modes disclosed herein and/or to toggle between drive modes.

While particular combinations of controls (e.g., buttons, joysticks, etc.) and indicators are described above with respect to the controller 6610, different arrangements and combinations of controls and/or indicators may be used to perform the functions described above.

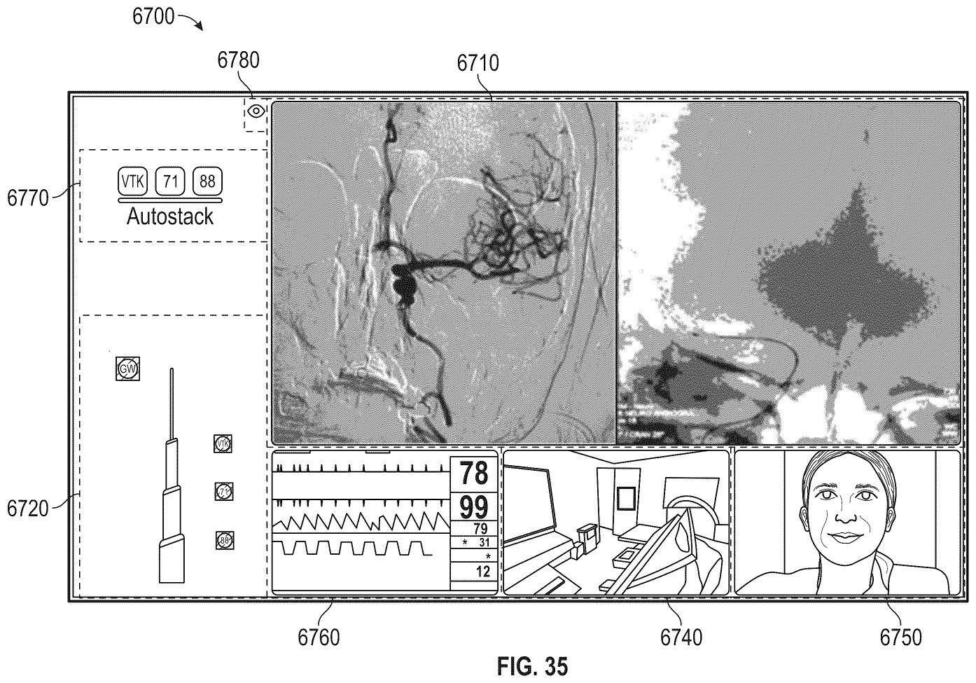

FIG. 35 is another embodiment of a user interface 6700 for facilitating control of the control mechanisms for interventional devices described herein. The user interface 6700 can include any of the same or similar features and/or functions as the user interface 5400 and/or the use interface 6500.

The user interface 6700 can include a plurality of windows. For instance, the user interface 6700 can include an imaging window 6710, an instrument window 6720, a first video feed window 6740, a second video feed window 6750, a patient vitals window 6760, and/or a toggle indicator window or status indicator window 6770. In some cases, only some of the windows shown in FIG. 35 are displayed. In some embodiments, the user interface 6700 can be displayed on a capacitive screen, a resistive screen, a touch pad, or other touch-based sensing device.

Determining whether the feed on a window, such as the patient feed and/or fluoroscopy, is live can be important during an interventional procedure. Thus, the user interface 6700 can include an indicator 6780. The indicator 6780 can provide an indication regarding whether the feeds of any fluoroscopy windows on the user interface 6700 are live. For example, the indicator 6780 may be displayed on the user interface to indicate that the feed of one or more fluoroscopy windows of the user interface 6700 are live. In some cases, the indicator 6780 is not displayed when none of the feeds on the fluoroscopy windows are live. Additionally, or alternatively, a perimeter of each window of the user interface 6700 can change appearance (e.g., color, pattern, etc.) to indicate that the feed on the window is live (e.g., to provide a contrast to any window showing a feed that is not live at the same time).

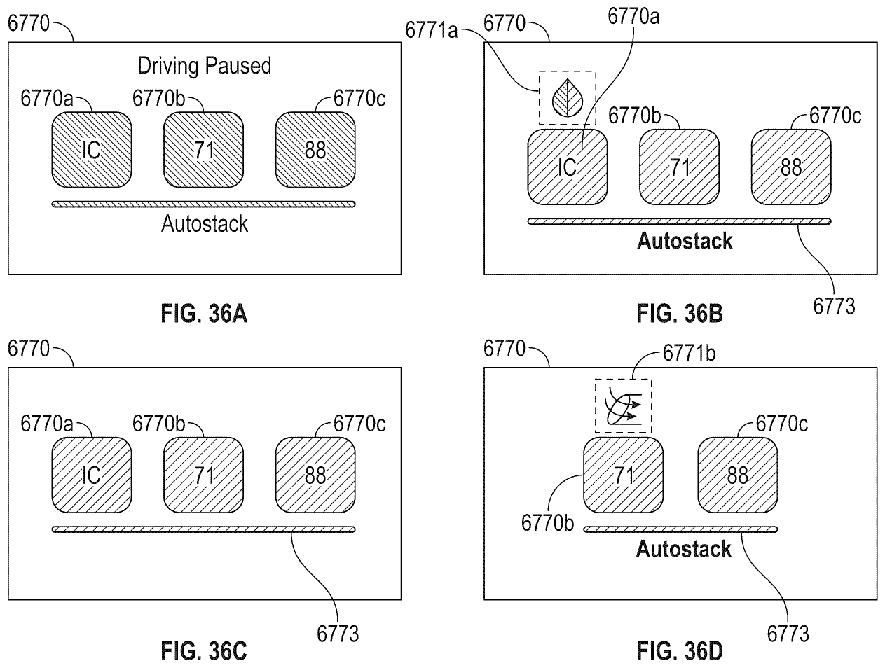

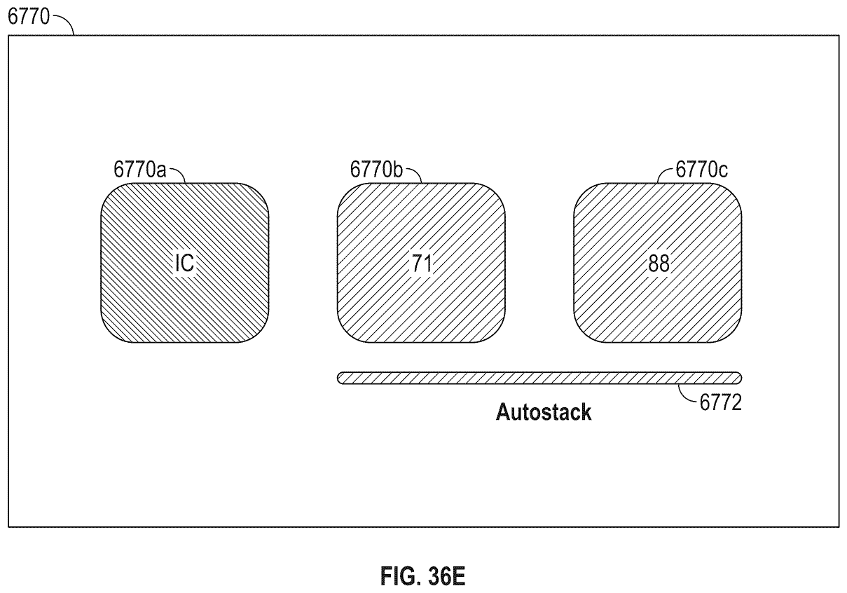

As previously described, users can toggle between individual hub assemblies and/or interventional devices controlled by a control, such as a joystick 6334, 6434, and/or 6634 by pressing a button on the control mechanism, such as button 6353, 6453, and/or 6653. When toggling between hub assemblies and/or interventional devices, it may be important to identify which of the hub assemblies and/or interventional devices is currently linked to the control. As shown in FIGS. 36A-36D, the status indicator window 6770 can provide a visual indication to identify which hub assemblies and/or interventional devices are currently linked to the control. For example, the status indicator window 6770 may include icons 6770a, 6770b, 6770c, each corresponding to one of the hub assemblies and/or interventional devices available to be linked to a control, such as a joystick 6334, 6434, and/or 6634. For example, as described herein, in some embodiments, a joystick 6334, 6434, or 6634 may be linked, simultaneously and/or individually, to a plurality of interventional devices (e.g., an insert or access catheter, a guide catheter, and/or a procedure catheter, etc.) during a procedure.

The first icon 6770a may correspond to a hub assembly, such as an insert catheter hub assembly, and/or interventional device, such as an insert catheter. The second icon 6770b may correspond to a hub assembly, such as a procedure catheter hub assembly, and/or interventional device, such as a procedure catheter. The third icon 6770c may correspond to a hub assembly, such as a guide catheter hub assembly, and/or interventional device, such as a guide catheter.

In some embodiments, the status indicator window 6770 can include a message area which may provide additional information regarding the status of the hub assemblies and/or interventional devices associated with the icons. For example, as shown in FIG. 36A, the message area states “Driving Paused” to indicate that the drive system is in a “paused state” meaning that the user is prevented from driving the interventional devices. In other embodiments, the message area may provide drive fault notifications or indicate when a hub assembly has reached a maximum position in a particular direction. The message area may be positioned above the icons.

In some embodiments, the status indicator window 6770 may display the icon corresponding to any hub assembly and/or interventional device currently linked to a particular control such as a joystick 6334, 6434, or 6634, (such that manipulation of the control causes a corresponding movement of those hub assemblies and/or interventional devices) in a first color, texture, or pattern, and display the icon corresponding to any unlinked hub assembly and/or interventional device in a second color, texture, or pattern. For example, in some embodiments, the icons corresponding to hub assemblies and/or interventional devices currently linked to the control may appear illuminated, and the icons corresponding to hub assemblies and/or interventional devices not linked to the control may appear dark.

In some embodiments, each of the icons may also be displayed in an unlinked and/or inactive state when driving using the corresponding control is not taking place or is prevented. For example, the icons of the status indicator window 6770 shown in FIG. 36B can indicate that all corresponding hub assemblies and/or interventional devices are linked and/or active. In contrast, FIG. 36A depicts the icons in a darker appearance, which may indicate that driving is not taking place or is prevented.

In some embodiments, the status indicator window 6770 may show icons only corresponding to the hub assemblies that currently have an interventional device coupled thereto. For example, FIG. 36D illustrates only icons 6770b and 6770c, indicating that, when using a drive system having three hub assemblies that can be linked with the control, only two of the hub assemblies have an interventional device coupled thereto.

In some embodiments, the status indicator window may include an additional visual indicator 6773 identifying the icons corresponding to hub assemblies and or interventional devices that are linked to the control. As shown, the indicator 6773 may be in the form of a horizontal line positioned below the icons corresponding to linked interventional devices.

In some alternative embodiments, the status indicator window 6770 may include icons corresponding to only each hub assembly and/or interventional device that is linked with a control, such as a joystick 6334, 6434, or 6634, such that manipulation of the control causes a corresponding movement of those hub assemblies and/or interventional devices. In such embodiments, icons corresponding to unlinked hub assemblies and/or interventional devices may not be displayed. In some embodiments, the icons may be displayed with a first visual appearance (e.g., a first color, texture, and/or pattern) to indicate that the corresponding devices are actively being driven and a second visual appearance (e.g., a second color, texture, and/or pattern) to indicate that they are not actively being driven.

In some cases, the status indicator window 6770 can provide an indication of the operational status of the corresponding hub assemblies and/or interventional devices for each of the icons. For example, an interventional device marker may be displayed in association with (e.g., next to, overlaying, partially overlaying) with an icon representing a particular hub assembly and/or interventional device to provide an indication of an operational status thereof. For example, as shown in FIG. 36B, the status indicator window 6770 can display an interventional device marker 6771a. The interventional device marker 6771a can include a symbol and/or a picture (e.g., a water drop) to indicate, for example, that contrast injection and/or other fluid delivery is available at the interventional device associated with an icon (e.g., the first icon 6770a). As another example, and as shown in FIG. 36D, the status indicator window 6770 can display an interventional device marker 6771b. The interventional device marker 6771b can include a symbol and/or a picture to indicate, for example, that aspiration is available and/or active at the interventional device associated with an icon (e.g., the second icon 6770b). The interventional device markers may be any of the markers shown and described with respect to FIGS. 9-20.

The status indicator window 6770 can provide messages and/or warnings regarding the operational status of the corresponding hub assemblies and/or interventional devices for each of the icons. In some embodiments, the status indicator window 6770 can provide an indication that one or more of the icons are in a particular drive mode. For example, as shown in FIGS. 36A, 36B, 36D, and 36E, the status indicator window 6770 may display a message indicating whether “Autostack,” which is described in relation to FIGS. 24A-24C, is active. For example, as shown in FIG. 36B, the line indicator 6772 extending below the icons 6770a, 6770b, and 6770c, can indicate that the interventional devices associated with the icons 6770a, 6770b, and 6770c, are linked to each other (e.g., configured to advance and/or retract simultaneously), and the “Autostack” message can indicate that the linked devices are configured to operate in an auto stacking drive mode as described herein. As shown in FIGS. 36D and 36E, the line indicator 6772 extending below the icons 6770b and 6770c can indicate that the interventional devices associated with the icons 6770b, and 6770c are linked to each other (e.g., configured to advance and/or retract simultaneously), and the “Autostack” message can indicate that the linked devices are configured to operate in an auto stacking drive mode as described herein. In FIG. 36E, the line indicator 6772 does not extend below the icon 6770a, thus indicating that although an interventional device associated with the icon 6770a is installed, the interventional device is not linked to the interventional devices associated with icons 6770b and 6770c. As shown in FIG. 36E, the “Autostack” message can indicate that the linked devices associated with the icons 6770b and 6770c are configured to operate in an autostacking drive mode. As shown in FIG. 36C, the lack of the “Autostack” message can provide an indication that “Autostack” is not active.

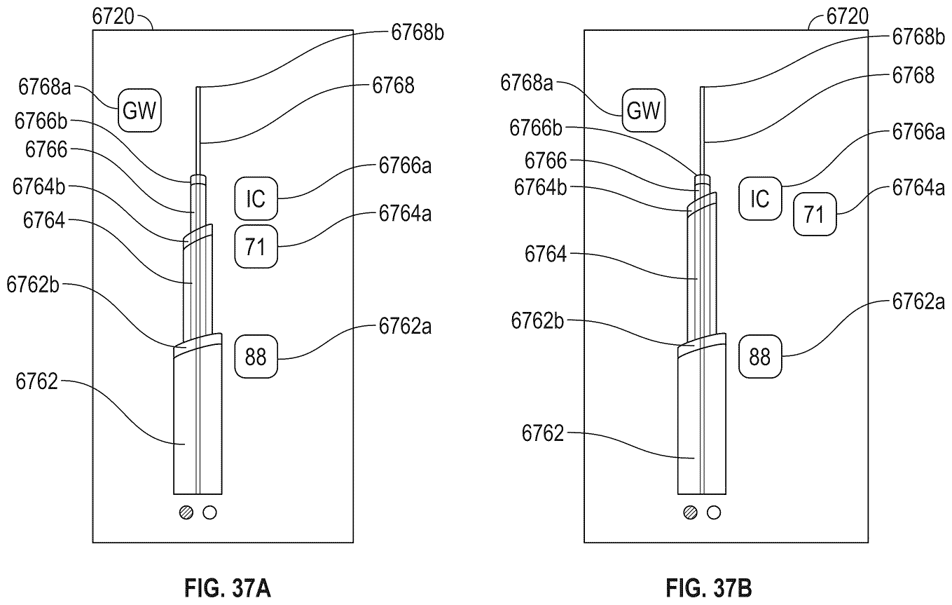

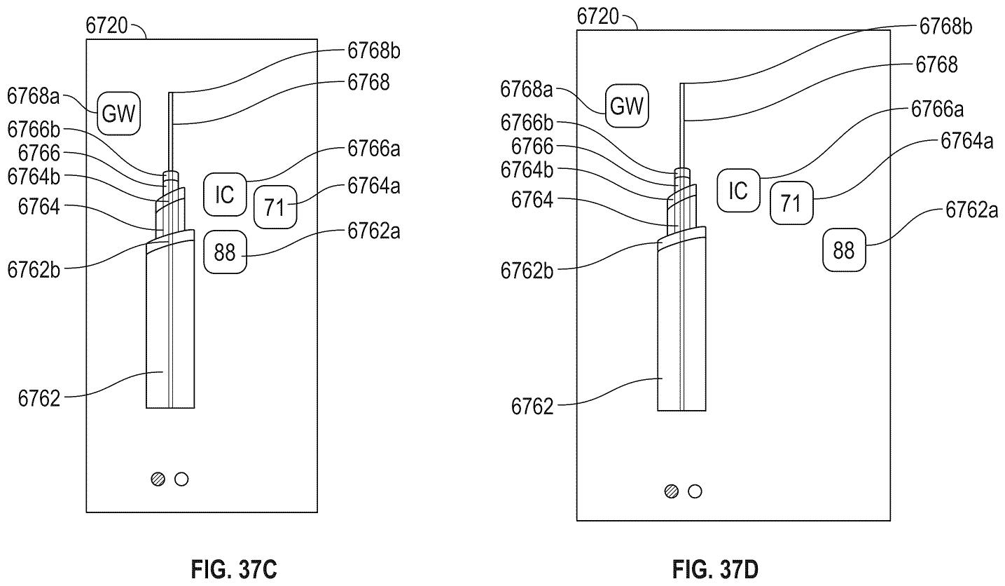

The instrument window 6720 may include any of the same features and/or functions as the instrument window 5420 or the instrument window 6520. The instrument window 6720 can provide a visual indication of the status of one or more interventional devices. For example, the instrument window 6720 can display what interventional devices, if any, are active and/or associated to respective hubs of the drive system (e.g., drive system 18). This can beneficially allow clinicians to easily identify which interventional devices are available and the position of the interventional devices relative to each other. As shown in FIGS. 37A-37D, the instrument window 6720 can include a plurality of interventional device representations 6762, 6764, 6766 and 6768. In some embodiments, each of the plurality of interventional device representations can represent a particular interventional device coupled to a drive table in communication with the user interface (e.g., via a control system or control console). In certain embodiments, the control system or control console may receive information, related to the particular interventional devices (e.g., identify information, shape and/or size information, axial and/or rotational position of the interventional devices, information regarding an order of devices, axial and/or rotational velocity of the interventional devices, magnitudes and/or directions of axial and/or rotational movement, etc.) via a sensor system including one or more sensors (e.g., sensors 3135, sensors 3130a). The received information can be used (e.g., by one or more hardware processors) to generate a user interface providing information about the particular interventional devices. For example, the identify information can be used (e.g., by one or more hardware processors) to provide an indication of the particular interventional devices represented by the interventional device representations (e.g., by generating a user interface including the identify information).

In certain embodiments, each of the interventional device representations 6762, 6764, 6766 and 6768 may have visual characteristics that correspond to characteristics of the interventional devices to which they correspond. For example, each interventional device representation 6762, 6764, 6766 and 6768 may be shaped and/or sized (e.g., on a scaled or proportional basis) to correspond to the shape and/or size of the interventional device to which it corresponds or a portion of the interventional device to which it corresponds (e.g., a distal section). In some embodiments, the interventional device representations 6762, 6764, 6766 and 6768 may be displayed in relative positions corresponding to the relative positions of the interventional devices to which they correspond. For example, a distance between the distal ends of two interventional device representations may correspond (e.g., on a 1:1 or scaled basis) to a distance between the distal ends of the two interventional devices to which the interventional device representations correspond.

Each of the plurality of interventional device representations 6762, 6764, 6766 and 6768 can include a marker 6762a, 6764a, 6766a, 6768a. The markers 6762a, 6764a, 6766a, 6768a can provide an indication of the specific interventional devices each of the plurality of interventional device representations 6762, 6764, 6766 and 6768 are associated with. For example, each marker can include a character or set of characters identifying the interventional device its respective interventional device representation is associated with.

For example, as shown in FIGS. 37A-37B, the marker 6762a can provide an indication (e.g., the label “88”) that the interventional device representation 6762 is associated with a guide catheter (e.g., guide catheter 31), which may be a guide catheter having a 0.088 inch inner diameter. As another example, the marker 6764a can provide an indication (e.g., the label “71”) that the interventional device representation 6764 is associated with a procedure catheter (e.g., catheter 120), which may be a procedure catheter having a 0.071 inch inner diameter. In some embodiments, the marker 6766a can provide an indication (e.g., the label “IC” as shown in FIGS. 37A-37D) that the interventional device representation 6766 is associated with an insert catheter (e.g., insert or access catheter 2902). In some embodiments, the marker 6766a can provide an indication that the interventional device representation 6766 is associated with a second procedure catheter (e.g., catheter 124), which may have a 0.035 inch inner diameter. The marker 6768a can provide an indication (e.g., the label “GW”) that the interventional device representation 6768 is associated to a guidewire (e.g., guidewire 27 or guidewire 2907).

Each of the interventional device representations 6762, 6764, 6766 and 6768 can include a visual indication 6762b, 6764b, 6766b, and 6768b of a distal end of the interventional device representation. Each of the visual indications 6762b, 6764b, 6766b, and 6768b can include a shape corresponding to a distal shape of the interventional device associated with the interventional device representations 6762, 6764, 6766 and 6768.

The instrument window 6720 can provide a visual indication of the axial position of the interventional device representations 6762, 6764, 6766 and 6768 with respect to each other. This can beneficially allow clinicians to assess the available range of motion for each interventional device associated with the interventional device representations 6762, 6764, 6766, 6768. The interventional device representations 6762, 6764, 6766, 6768 can be displayed so that a position of a distal end of each of the interventional device representations is shown. The visual indications 6762b, 6764b, 6766b, and 6768b and/or the markers 6762a, 6764a, 6766a, 6768a can provide an indication of the position of the distal ends of the interventional devices corresponding to the interventional device representations with respect to each other. For example, the marker 6768a of the interventional device representation 6768 can provide an indication that the distal end of the interventional device associated with the interventional device representation 6768 (e.g., a guidewire) extends beyond a distal end of the interventional devices associated with the interventional device representations 6762, 6764 and/or 6766.

The markers 6762a, 6764a, 6766a, 6768a may provide an indication of relative distances between the interventional devices corresponding to the interventional device representations 6762, 6764, 6766, 6768 relative to each other. In some embodiments, as the interventional devices move relative to one another, the interventional device representations 6762, 6764, 6766, 6768 and/or their corresponding markers 6762a, 6764a, 6766a, 6768a can move relative to each other to indicate the position of each interventional device. For example, as the interventional device associated to the interventional device representation 6764 is advanced, the position of the interventional device representation 6764 with respect to the interventional device representations 5462, 6766, 6768 may change to show the interventional device extended farther away from the interventional device representations 6762, 6766, 6768, as shown in FIG. 37B. Additionally, or alternatively, the position of the marker 6764a with respect to the other markers 6762a, 6766a, 6768a may change.

In some cases, as the tip of any interventional device approaches the tip of another interventional device, the marker 6762a, 6764a, or 6766a associated with the tip approaching the tip of the other interventional device may start moving along a different vertical axis. This can allow two or more markers 6762a, 6764a, 6766a to be displayed at least partially side to side when the tips of their corresponding devices are aligned or almost aligned. For example, as shown in FIG. 37A, when none of the distal tips of the interventional devices corresponding to catheters are aligned, their corresponding markers 6762a, 6764a, 6766a, may be displayed in a single vertical axis. As the tip of an interventional device associated with marker 6764a gets closer to the tip of the interventional device associated with marker 6766a, as shown in FIGS. 37B and 37C, the markers 6762a, 6764a, 6766a may be displayed in two vertical axes. As shown, the marker 6764a is shown along a separate vertical axis from the markers 6766a and 6762a in FIGS. 37B and 37C. The marker 6764a is shown lateral to the marker 6766a, which provides an indication that the distal tips associated with the markers 6766a, and 6764a, are aligned or almost aligned. As the tip of three interventional devices get closer to each other, as shown in FIG. 37D, the markers 6762a, 6764a, 6766a may be displayed in three vertical axes. This can provide an indication that the distal tips associated with three markers 6762a, 6764a, 6766a are aligned or almost aligned. In certain embodiments, information regarding the positions of the interventional devices and/or movement of the interventional devices (e.g., axial and/or rotational velocity, magnitude and/or direction of movement, etc.) may be received from one or more sensors (e.g., sensors 3135 and/or sensors 3130a) of a sensor system.

In some embodiments, one or more of the representations 6762, 6764, 6766, 6768 and/or their corresponding markers 6762a, 6764a, 6766a, 6768a may change appearance to provide an indication of a status of the corresponding interventional device or operation thereof (for example, as described with respect to FIGS. 9-21). For example, one or more of the representations 6762, 6764, 6766, 6768 and/or their corresponding markers 6762a, 6764a, 6766a, 6768a may provide an indication that a corresponding device is linked to a control or is not linked to a control. One or more of the representations 6762, 6764, 6766, 6768 and/or their corresponding markers 6762a, 6764a, 6766a, 6768a may provide an indication that a corresponding interventional device is performing a contrast injection or aspiration. One or more of the representations 6762, 6764, 6766, 6768 and/or their corresponding markers 6762a, 6764a, 6766a, 6768a may provide a warning of an error or of a possible collision of hubs of corresponding interventional devices. One or more of the representations 6762, 6764, 6766, 6768 and/or their corresponding markers 6762a, 6764a, 6766a, 6768a may provide an indication that a device is installed (e.g., coupled to a hub) or not installed (e.g., not coupled to a hub).

The user interface 6700 can, in some cases, display the position of each hub assembly along a drive table (e.g., relative to a drive surface and/or main body on which the hub assemblies are located).

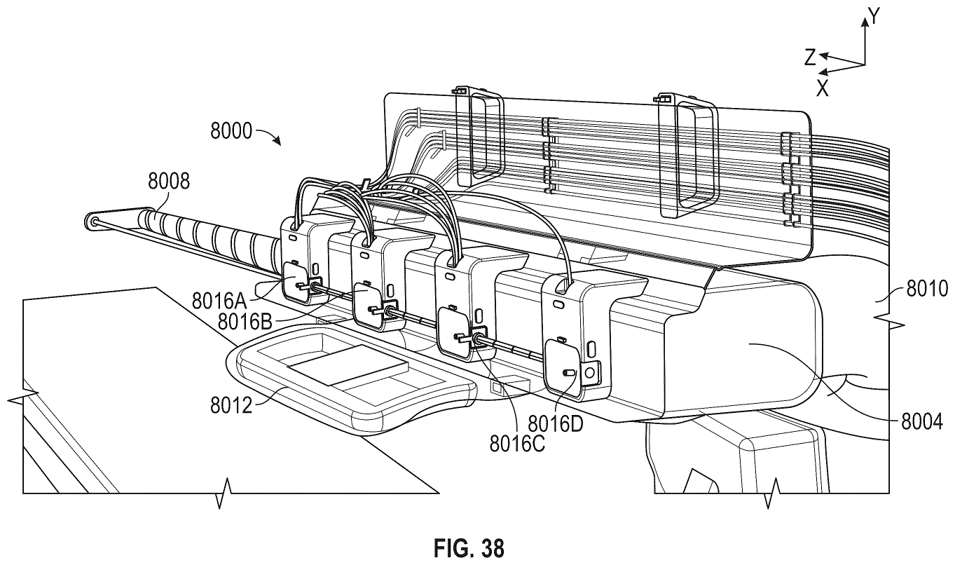

For example, FIG. 38 illustrates an example of a drive table 8000. The drive table 8000 can include any of the same and/or similar features as any of the drive tables described herein and vice versa. The drive table 8000 can include a main body 8004 (which may also be referred to as a shuttle). In some embodiments, the drive table 8000 can include one or more planar support surfaces 8006A-B. In some embodiments, the drive table 8000 can include an extendable or telescoping member 8008 (also referred herein to as a hockey stick, a telescoping arm, or a telescoping support). The drive table 8000 can be further coupled to a base 8010. In some embodiments, the drive table 8000 can further include a handle 8012. The drive table 8000 can be configured to position a drive assembly proximal to an access point on a patient.

The drive table 8000 can include a plurality of hub assemblies. The plurality of hub assemblies can include a first hub assembly 8016A, a second hub assembly 8016B, a third hub assembly 8016C, and/or a fourth hub assembly 8016D. Each of the plurality of hub assemblies can be configured to translate along the one or more planar support surfaces 8006A-B of the drive table 8000 or a drive surface placed over one or more of the planar support surfaces 8006A-B of the drive table (e.g., a drive surface of a sterile barrier placed over one or more of the planar support surfaces 8006A-B). In some embodiments, the plurality of hub assemblies can be magnetically coupled to a corresponding carriage or hub adapter of the drive system located within the main body 8004. Each hub assembly of the plurality of hub assemblies can move relative to the main body 8004. For example, each hub assembly of the plurality of hub assemblies can be moved in a distal direction thereby advancing the plurality of hub assemblies and their corresponding interventional devices into the patient. Each hub assembly of the plurality of hub assemblies can be moved in a proximal direction thereby retracting the plurality of hub assemblies and their corresponding interventional devices away from the patient.

The telescoping arm or telescoping member 8008 can be a longitudinal body configured to extend from and/or retract into the main body 8004. In some embodiments, the telescoping member 8008 can be a support bracket. In some embodiments, the telescoping member 8008 may be configured to support one or more interventional devices at a position proximal to a patient. In some embodiments, the telescoping member 8008 can be coupled to an access sheath (e.g., a femoral access sheath) at a patient access point. In some cases, the main body 8004 can be advanced and/or retracted over the telescoping member 8008. This can allow the position of the main body 8004 relative to a patient to be adjusted. For example, the main body 8004 can be advanced over the telescoping member 8008 to bring the main body 8004 closer to the patient thereby advancing the plurality of hub assemblies and their corresponding interventional devices into the patient. The main body 8004 can be retracted over the telescoping member 8008 to bring the main body 8004 away from the patient thereby retracting the plurality of hub assemblies and their corresponding interventional devices from the patient. Additional details regarding drive tables are disclosed in U.S. Provisional Application Ser. No. 63/656,547, entitled DRIVE TABLE, filed Jun. 5, 2024, the entirety of which is hereby expressly incorporated by reference herein. Additional details regarding drive tables are disclosed in U.S. patent application Ser. No. 18/524,879, entitled “ROTATABLE DRIVE TABLE,” filed Nov. 30, 2023, the entirety of which is hereby expressly incorporated by reference herein.

In some embodiments, the arm or member 8008 may not telescope within the main body 8004. The body 8004 may move relative to the arm or member 8008 in an alternative manner. For example, the arm or member 8008 may include a track positioned exterior to the main body 8004 along which the main body 8004 can translate.

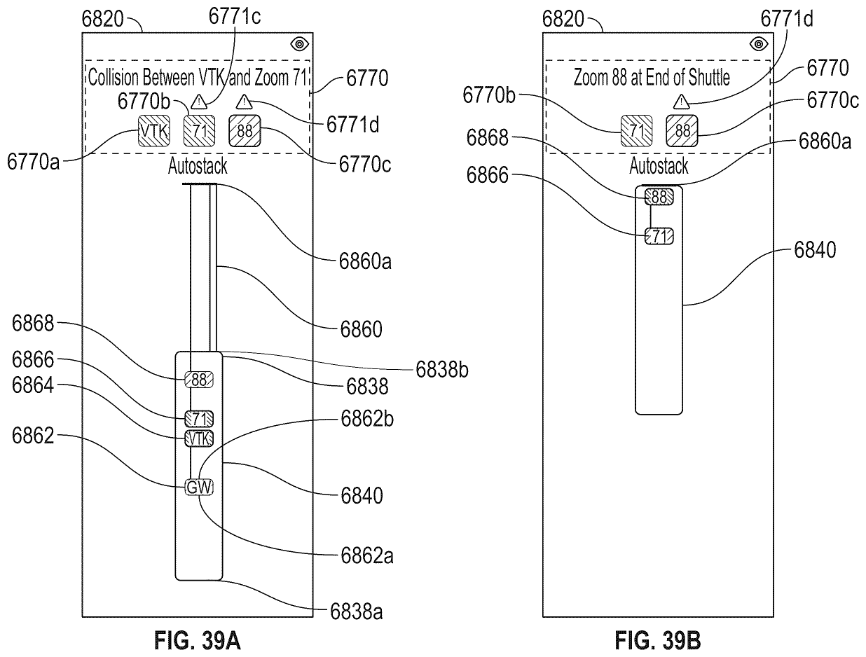

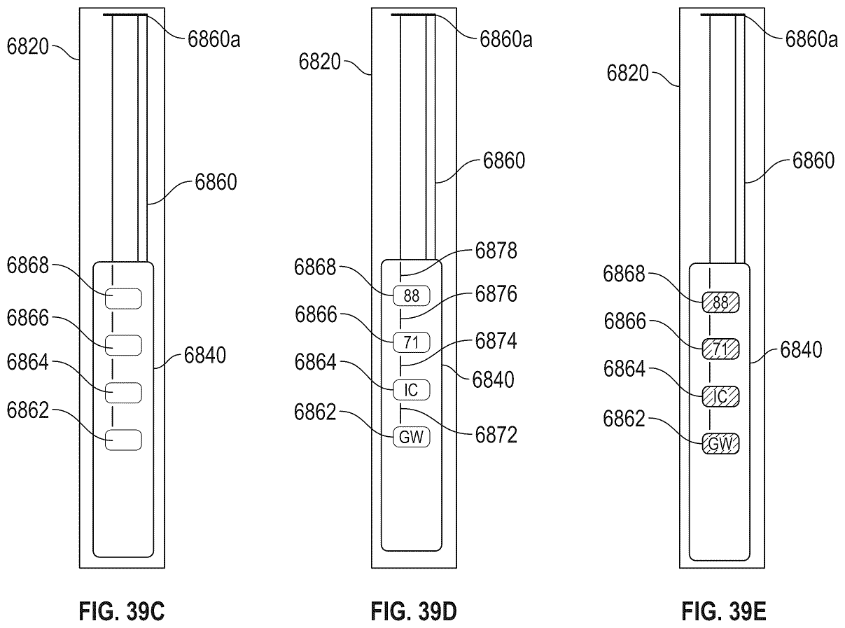

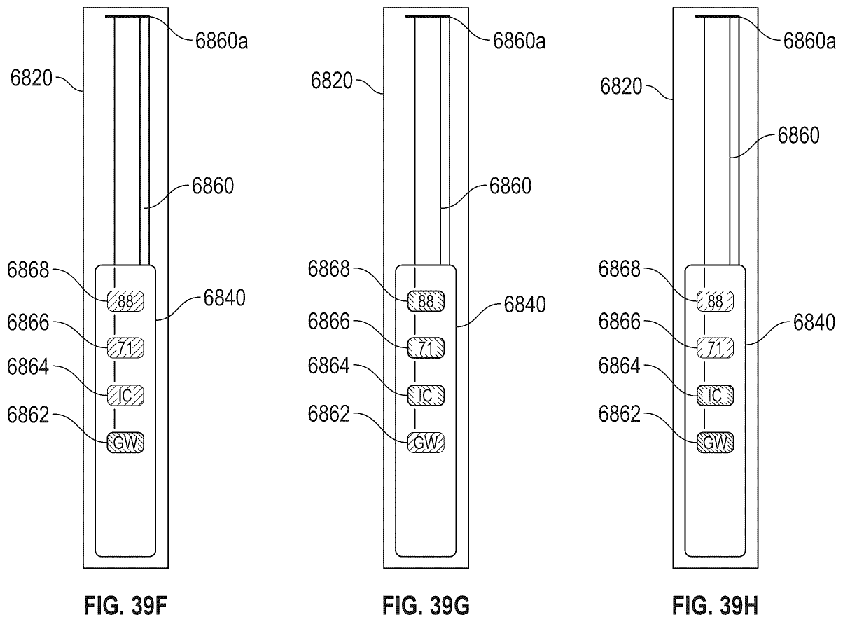

It may be advantageous to identify the location of the main body 8004 relative to the patient and/or the telescoping member 8008, and/or the position of the plurality of hub assemblies relative to each other and/or relative to the main body 8004 during an interventional procedure. In some embodiments, the user interface can display a drive table window 6820, as shown in FIGS. 39A-39H. The drive table window 6820 can provide visual representations of one or more components of the drive table and the hub assemblies to identify the relative locations thereof (e.g., of the main body 8004, the telescoping member 8008, and or hub assemblies 8016A-8016D.

In some embodiments, the user interface may toggle between the instrument window 6720 and the drive table window 6820. Users can toggle between the instrument window 6720 and the drive table window 6820, for example, by pressing a button on the controller and/or via the user interface 6700. In some cases, the user interface 6700 can automatically switch from showing the instrument window 6720 to showing the drive table window 6820, or vice versa, in response to the occurrence of a trigger event (also referred hereon to as a control system condition). In some embodiments, the user interface may automatically show the drive table window 6820 in response to a triggering event (for example, if a hub assembly is detected to be approaching another hub assembly at a distance that may result in a collision, or if a hub assembly is approaching a distal or proximal end of a drive table). In some embodiments, the user interface may automatically show the instrument window 6720 in response to a triggering event.

In some embodiments, the drive table window 6820 can provide a visual indication of the status of one or more interventional devices and/or a drive table. For example, the drive table window 6820 can display what interventional devices, if any, are active and/or associated to respective hubs assemblies of the drive system (e.g., drive system 18). This can beneficially allow clinicians to easily identify which interventional devices are available and the position of the interventional devices relative to each other, the main body, and/or the telescoping member. The drive table window 6820 may provide a visual indication of which hub assemblies and/or interventional devices are currently linked to a control of a controller so that movement of the control will cause a corresponding movement of the hub assemblies.

As shown in FIGS. 39A-39H, in some embodiments, the drive table window 6820 can include a plurality of hub assembly representations 6862, 6864, 6866 and 6868. The hub assembly representations may each provide information, as described in further detail herein, regarding a corresponding hub assembly (e.g., the hub assemblies 8016A-D). As described herein (e.g., with respect to FIG. 33), in certain embodiments, a hub assembly may include a separate mount and a hub that is couplable to the mount. In such embodiments, the hub assembly representations may provide information relative to a mount positioned on the drive table when no hub is coupled thereto (e.g., axial position information, axial velocity information, an indication that a hub is not coupled to a mount). In some embodiments, the hub assembly representations may be referred to as mount representations.

The drive table window 6820 can provide a visual representation of the one or more interventional devices associated with the hub assemblies 8016A-8016D and/or the interventional device stack including the interventional devices associated with the hub assemblies 8016A-8016D. For example, as shown in FIG. 39D, the drive table window can include an interventional device representation for each of the hub assembly representations 6862, 6862, 6866, and 6868. The interventional device representations 6872, 6874, 6876, and 6878, can correspond to the interventional devices associated with the hub assembly representations 6862, 6862, 6866, and 6868, respectively.

Each of the plurality of hub assembly representations 6862, 6864, 6866 and 6868, can include a proximal end and a distal end. For example, as shown in FIG. 39A, the first hub assembly representation 6862 can include a proximal end 6862a and a distal end 6862b,

In some embodiments, the drive table window 6820 can include a drive table representation 6838. The drive table representation 6838 can provide a visual representation of a corresponding drive table (e.g., drive table 8000). The drive table representation 6838 can include a first end 6838a and a second end 6838b.

In some embodiments, the drive table window 6820 can include a drive table body or drive surface representation 6840. The drive surface representation can provide a visual representation of a corresponding drive surface or a drive table body along which drive surface is positioned or attached (e.g., main body 8004). In some embodiments, the drive surface representation 6840 may be the same as or generally the same as the drive table representation 6838. In other embodiments, the drive table may include additional components that are separately represented on the user interface (e.g., as shown in FIGS. 39A-39H).

In some embodiments, the drive table window 6820 can include a telescoping arm representation or telescoping member representation 6860. The telescoping member representation 6860 can provide a visual representation of a telescoping member (e.g., telescoping member 8008). In some embodiments, the drive table representation 6838 can include the drive surface representation 6840 and the telescoping member representation 6860.

In some embodiments, each of the plurality of hub assembly representations can represent a particular hub assembly and/or mount coupled to a drive table in communication with the user interface (e.g., via a control system or control console). The hub assembly representations can provide visual indications of the positions of the hub assemblies (or mounts) relative to each other and/or to the drive table and/or telescoping member.

The drive surface representation 6840 and/or the telescoping member representation 6860 can provide visual indications of the position of the drive surface (e.g., the main body 8004) and the telescoping member (e.g., the telescoping member 8008) relative to each other and/or to the hub assemblies (or mounts).

In certain embodiments, the control system or control console may receive information, related to the particular hub assemblies and/or mounts (e.g., identify information of the hub assembly, mount, and/or interventional device coupled to each hub assembly; whether or not a hub and/or interventional device is coupled to a particular mount; shape and/or size information; axial and/or rotational position of the hub assemblies, mounts, and/or interventional devices; information regarding an order of hub assemblies, mounts, and/or interventional devices; axial and/or rotational velocity of the hub assemblies, mounts and/or interventional devices; magnitudes and/or directions of axial and/or rotational movement of the hub assemblies, mounts, and/or interventional devices, etc.) via a sensor system including one or more sensors (e.g., sensors 3135, sensors 3130a). In certain embodiments, the control system or control console may receive information related to the drive table via a sensor system including one or more sensors (e.g., sensors 3135, sensors 3130a). For example, the control system or control consol may receive information related to the main body (axial position, axial velocity, magnitudes and/or directions of axial movement, etc.), and/or telescoping member (axial position, axial velocity, magnitudes and/or directions of axial movement, etc.), via a sensor system including one or more sensors (e.g., sensors 3135, sensors 3130a). The received information can be used (e.g., by one or more hardware processors) to generate a user interface providing information about the particular hub assemblies, the mounts, the interventional devices, the drive table, the main body, and/or the telescoping member. For example, the identify information can be used (e.g., by one or more hardware processors) to provide an indication of the particular hub assembly represented by a particular hub assembly representation or the particular mount represented by a particular hub assembly representation if no hub is attached to the mount (e.g., by generating a user interface including the identify information).

The drive table representation 6838 can include visual characteristics that correspond to the visual characteristics of a drive table (e.g., drive table 8000). For example, the drive e table representation 6838 may be shaped and/or sized (e.g., on a scaled or proportional basis) to correspond to the shape and/or size of the drive table to which it corresponds (e.g., drive table 8000).

The drive surface representation 6840 can include visual characteristics that correspond to the visual characteristics of a drive surface or drive table body (e.g., main body 8004 or a surface thereof or positioned thereon). For example, the drive surface representation 6840 may be shaped and/or sized (e.g., on a scaled or proportional basis) to correspond to the shape and/or size of the drive surface or drive table body to which it corresponds (e.g., main body 8004 or a surface thereof or positioned thereon).

In some embodiments, the hub assembly representations 6862, 6864, 6866, and 6868 6840 can include visual characteristics that correspond to the visual characteristics of hub assemblies and/or mounts to which they correspond (e.g., hub assemblies 8116A-8116D). For example, the hub assembly representations 6862, 6864, 6866, and 6868 may be shaped and/or sized (e.g., on a scaled or proportional basis) to correspond to the shape and/or size of the hub assemblies to which they correspond (e.g., hub assemblies 8116A-8116D).

In some embodiments, the hub assembly representations 6862, 6864, 6866, and 6868 may be displayed in relative positions corresponding to the relative positions of the hub assemblies to which they correspond. For example, a distance between the distal ends of two hub assembly representations may correspond (e.g., on a 1:1 or scaled basis) to a distance between the distal ends of the two hub assemblies to which the hub assembly representations correspond.

In some embodiments, each hub assembly representation and the drive table representation 6838 may be displayed in relative positions corresponding to the relative positions of each hub assembly relative to the drive table to which the hub assembly representations and the drive table representation correspond. In some embodiments, each hub assembly representation and the drive surface representation 6840 may be displayed in relative positions corresponding to the relative positions of each hub assembly and the drive surface or drive table body to which the hub assembly representations and drive surface representations correspond. In some embodiments, each hub assembly representation and the telescoping member representation 6860 may be displayed in relative positions corresponding to the relative positions of each hub assembly and the telescoping member to which the hub assembly representations and the telescoping member representation 6860 correspond. In some embodiments, the drive surface representation 6840 and the telescoping member representation 6860 may be displayed in relative positions corresponding to the relative positions of the drive surface or drive table body and the telescoping member to which the drive surface representation 6840 and telescoping member representation 6860 correspond.

In some embodiments, a hub assembly representation 6862, 6862, 6864, 6866, or 6868 can provide a visual indication identifying an interventional device of the hub assembly corresponding to the hub assembly representation. For example, the representation 6868 can provide an indication (e.g., the label “88”) that a hub assembly associated with the hub assembly representation 6868 includes a guide catheter (e.g., guide catheter 31), which may be a guide catheter having a 0.088 inch inner diameter. As another example, the representation 6866 can provide an indication (e.g., the label “71”) that the hub assembly associated with the hub assembly representation 6866 includes a procedure catheter (e.g., catheter 120), which may be a procedure catheter having a 0.071 inch inner diameter. In some embodiments, the representation 6864 can provide an indication (e.g., the label “IC” as shown in FIGS. 37A-37D) that the hub assembly associated with the hub assembly representation 6864 includes an insert catheter (e.g., insert or access catheter 2902). In some embodiments, the representation 6864 can provide an indication that the hub assembly associated with the hub assembly representation 6864 includes a second procedure catheter (e.g., catheter 124), which may have a 0.035 inch inner diameter. The representation 6862 can provide an indication (e.g., the label “GW”) that the hub assembly associated with the hub assembly representation 6862 is associated with a guidewire (e.g., guidewire 27 or guidewire 2907).

In some cases, as the hub assemblies (or mounts) move relative to one another, the hub assembly representations 6862, 6864, 6866, 6868 can move relative to each other to indicate the position of each hub assembly (or mount). For example, if the hub assembly (or mount) associated with the hub assembly representation 6864 is advanced while the hub assemblies (or mounts) associated with the hub assembly representations 6862, 6866, and 6868 remain stationary, the position of the hub assembly representation 6864 with respect to the hub assembly representations 6862, 6866, and 6868, will change.

In some embodiments, the hub assembly representations 6862, 6864, 6866, 6868 may move relative to a fixed point of reference within the drive table window 6820 in a manner corresponding to the movement of the corresponding hub assemblies relative to a fixed point of reference in the operating room (e.g., a patient access point, an operating table, etc.). In some embodiments, the drive table representation 6838 may move relative to a fixed point of reference within the drive table window 6820 in a manner corresponding to the movement of the corresponding drive table relative to a fixed point of reference in the operating room (e.g., a patient access point, an operating table, etc.). In some embodiments, the drive surface representation 6840 may move relative to a fixed point of reference within the drive table window 6820 in a manner corresponding to the movement of the corresponding drive surface or drive table body relative to a fixed point of reference in the operating room (e.g., a patient access point, an operating table, etc.). In some embodiments, the telescoping member representation 6860 may may move relative to a fixed point of reference within the drive table window 6820 in a manner corresponding to the movement of the corresponding drive surface or drive table body relative to a fixed point of reference in the operating room (e.g., a patient access point, an operating table, etc.).

In some embodiments, as the drive surface or drive table body associated with the drive surface representation 6840 is advanced or retracted along the telescoping member associated with the telescoping member representation 6860, the position of the drive surface representation 6840 relative to the telescoping member representation 6860 may change accordingly. For example, when the drive surface or drive table body (e.g., main body 8004) is at least partially retracted (e.g., the drive surface or drive table body is positioned away from a distal end of the telescoping member), the drive surface representation 6840 can be separated from a distal end 6860a of the telescoping member representation 6860, as shown in FIG. 39A. The separation between the drive surface representation 6840 and the distal end 6860a can provide a visual indication of the separation of between the drive surface or drive table body and the distal end of the telescoping member and/or that the drive surface or drive table body can be further advanced along the telescoping member. In some embodiments, the position of the distal end 6860a when fully extended can correspond to a position of an access point of a patient (e.g., correspond to a position of the distal end of the telescoping member when positioned at the patient access point).

As the drive surface or drive table body associated with the drive surface representation 6840 is advanced along the telescoping member, the position of the drive surface representation 6840 relative to the telescoping member representation 6860 can change to show the movement of the drive surface or drive table body relative to the telescoping member in real time or near real time. For example, the position of the drive surface representation 6840 on the drive table window 6820 can transition from a first animation (e.g., showing the position of the drive surface representation 6840 in FIG. 39A) to a second animation (e.g., showing the position of the drive surface representation 6840 in FIG. 39B).

In certain embodiments, the hub assembly representations 6862, 6864, 6866, 6868 may provide visual indications indicating that the corresponding hub assemblies are not coupled to the drive table. For example, as shown in FIG. 39C, the hub assembly representations 6862, 6864, 6866, 6868 may not display any symbols and/or text if a corresponding hub assemblies are not installed on the drive table. When a hub assembly is installed, a representation 6862, 6864, 6866, or 6868 associated with the hub assembly may display symbols or text (e.g., to indicate that identity of the hub assembly), as shown in FIG. 39D.

In some embodiments, one or more of the hub assembly representations 6862, 6864, 6866, and 6868 may provide visual indications indicating that interventional devices are not coupled to the mounts corresponding to the hub assembly representations (for example, by not displaying any symbols and/or text identifying the interventional devices). When an interventional device is installed to a mount associated with one of the representations 6862, 6864, 6866, 6868, that representation 6862, 6864, 6866, or 6868 may display symbols and/or text to indicate that an interventional device is now installed to the mount.

In some embodiments, one or more of the representations 6862, 6864, 6866, and/or 6868, may provide a visual indication (e.g., be displayed in first color, texture, or pattern), to indicate that the corresponding hub assembly associated with the representation is installed but not linked to a controller (e.g., controller 5310, 6310, 6410, 6610) and/or button/joystick of the controller. For example, in FIG. 39D, although a hub assembly is associated to each representation 6862, 6864, 6866, 6868, the hub assemblies have not been assigned to a controller and/or button/joystick of the controller. In some embodiments, one or more of the representations 6862, 6864, 6866, 6868, may provide a visual indication (e.g., be displayed in second color, texture, or pattern), to indicate that the corresponding hub assembly associated with the representation is assigned to a controller (e.g., controller 5310, 6310, 6410, 6610) and/or button/joystick of the controller. For example, in FIG. 39E, all the hub assemblies associated with representations 6862, 6864, 6866, 6868, have been assigned to a controller and/or button/joystick of the controller, thereby indicating that the corresponding interventional devices can be actuated by the controller.

In some embodiments, one or more of the representations 6862, 6864, 6866, and/or 6868, may provide a visual indication (e.g., be displayed in first color, texture, or pattern), to indicate that the corresponding interventional device associated with the representation is installed (e.g., its hub is coupled to the mount) but not to a controller (e.g., controller 5310, 6310, 6410, 6610) and/or button/joystick of the controller. In some embodiments, one or more of the representations 6862, 6864, 6866, 6868, may provide a visual indication (e.g., be displayed in second color, texture, or pattern), to indicate that the corresponding interventional device associated with the representation is assigned a controller (e.g., controller 5310, 6310, 6410, 6610) and/or button/joystick of the controller.

In some embodiments, one or more of the representations 6862, 6864, 6866, 6868, may provide a visual indication (e.g., be displayed in a third color, texture, or pattern), to indicate that the corresponding hub assembly (and/or interventional device) associated with the representation is actively being driven. For example, in FIG. 39F, the representation 6862 is shown in a third color, texture, and/or pattern to indicate that the interventional device associated with the representation 6862 is being driven (e.g., being advanced or retracted). As another example, in FIG. 39G, the representations 6864, 6866, 6868 are shown in a third color, texture, and/or pattern to indicate that the interventional devices associated with the representations 6864, 6866, 6868 are being driven (e.g., being advanced or retracted).

In some embodiments, one or more of the representations 6862, 6864, 6866, 6868, may provide a visual indication (e.g., be displayed in a fourth color, texture, or pattern), of a status of the hub assembly and/or interventional device associated with the particular representation. For example, in certain embodiments, a representation 6862, 6864, 6866, or 6868 may provide a visual indication of a fault condition or a warning condition related to its associated hub assembly and/or interventional device. For example, a representation 6862, 6864, 6866, or 6868 may provide a visual indication indicating that its corresponding hub assembly and/or interventional device is experiencing a high force or high resistance (e.g., above a defined threshold value). In some embodiments, a representation 6862, 6864, 6866, or 6868 may provide a visual indication indicating that is corresponding hub assembly is at its distal most position or nearing its distal most position (e.g., below a threshold distance to its distal most position) at a proximal or distal end of a drive surface. In some embodiments, a representation 6862, 6864, 6866, or 6868 may provide a visual indication that its corresponding hub assembly is colliding with or is close to colliding with another hub assembly. A hub assembly may be considered close to colliding with another hub assembly when a distance between the two hub assemblies is below a threshold value. For example, in FIG. 39A, the representations 6864 and 6866 are shown in a fourth color, texture, and/or pattern to indicate that their corresponding hub assemblies have collided. In FIG. 39B, the representation 6868 is shown in a fourth color, texture, and/or pattern to indicate that its corresponding hub assembly has reached a distal most position at a distal end of the drive surface (e.g., the second end 6838b of the drive table representation 6838). In FIG. 39H, the representations 6862 and 6864 are shown in a fourth color, texture, and/or pattern to indicate, for example, that their corresponding interventional devices and/or hub assemblies may be experiencing a high force and/or resistance.

In some embodiments, the user interface may automatically show the drive table window 6820 in response to a fault condition or warning condition related to one of the hub assemblies. For example, the user interface may automatically show the drive table window 6820 in response to a detection that two hub assemblies have collided or are near a collision. As described herein, a hub assembly may include a mount and a hub having an interventional device. In some embodiments, the interventional device may be considered part of the hub assembly. In other embodiments, the interventional device is considered separate from but coupled to the hub assembly. As described herein, collision can refer to contact between portions of adjacent hub assemblies (e.g., contact between adjacent mounts and/or hubs) other than the interventional devices, which as described herein, are concentrically positioned. A collision may occur when a distal end of a first hub assembly contacts a proximal end of a more distal second hub assembly (for example, when the first hub assembly is advancing distally). Similarly, a collision may occur when a proximal end of a first hub assembly contacts a distal end of a more proximal second hub assembly (for example, when the first hub assembly is advancing proximally).

The user interface may automatically show the drive table window 6820 in response to a detection that a hub assembly is at or near a proximal or distal end of a drive surface. In some cases, the drive table window 6820 can also include the status indicator window 6720. The user interface may automatically show the drive table window 6820 in response to detection that a hub assembly is experiencing a high force and/or high resistance. In some embodiments, the user interface may automatically return to the instrument window 6720 if the fault or warning condition ceases.

In some embodiments, the status indicator window 6770 may be shown anywhere on the user interface simultaneously with the drive table window 6820. In other embodiments, the status indicator window 6770 may be part of the drive table window 6820. In some embodiments, the status indicator window 6770 may be shown any time the drive table window 6820 is shown. In some embodiments, the status indictor window 6770 may be shown any time the instrument window 6720 is shown. In some cases, the status indicator window 6720 may indicate when two or more hub assemblies of the corresponding interventional devices are about to collide and/or have already collided. For example, FIG. 39A shows an embodiment of the status indicator window 6770 when the hubs associated with the hub assembly representations 6864 and 6866 have collided. These hub assemblies are also associated with icons 6770b and 6770c. As shown, the status indicator window 6770 includes a marker 6771c and a marker 6771d, each in the form of a warning sign. These markers 6771c and 6771d can provide an indication that there is a fault or warning condition associated with the hub assemblies or interventional devices associated with the icons 6770b and 6770c. The status indicator window 6770 may also include text indicating that a collision has occurred and identifying the hub assemblies or interventional devices associated with the collision. The status indicator window 6720 may indicate when one or more hub assemblies of the corresponding interventional devices are about to reach an end of the drive surface and/or have reached the end of the drive surface. For example, FIG. 39B shows an embodiment of the status indicator window 6770 when the hub assembly associated with the hub assembly representation 6868 has reached the end of the drive surface associated with the drive surface representation 6840. This hub assembly is also associated with icon 6770c. As shown, the status indicator window 6770 includes a marker 6771d, in the form of a warning sign. The marker 6771d can provide an indication that there is a fault or warning condition associated with the icon 6770c. The status indicator window 6770 may also include text indicating that a hub assembly has reached the end of the drive surface and identifying the particular hub assembly. In some embodiments, the status indicator window 6770 may be displayed automatically when a fault or warning condition is detected. In some embodiments, the user interface may stop showing the status indicator window 6770 on the drive table window 6820 when the fault or warning condition (e.g., near collision, collision, and/or hub position) ceases.

In some cases, the drive table window 6820 may provide a warning of an error or of a possible collision of hubs of corresponding interventional devices (e.g., when a hub abuts another hub). For example, each of the plurality of hub assembly representations 6862, 6864, 6866 and 6868 may provide a visual indication of a fault condition or a warning condition related to its associated hub assembly and/or interventional device. For example, each of the plurality of hub assembly representations 6862, 6864, 6866 and 6868 may provide a visual indication indicating that its corresponding hub assembly and/or interventional device is experiencing a high force or high resistance (e.g., above a defined threshold value). In some embodiments, each of the plurality of hub assembly representations 6862, 6864, 6866 and 6868 may provide a visual indication that its corresponding hub assembly is colliding with or is close to colliding with another hub assembly. A hub assembly may be considered close to colliding with another hub assembly when a distance between the two hub assemblies is below a threshold value. For example, in FIG. 39A, the representations 6864 and 6866 are shown in a fourth color, texture, and/or pattern to indicate that their corresponding hub assemblies have collided. In FIG. 39B, the representation 6868 is shown in a fourth color, texture, and/or pattern to indicate that its corresponding hub assembly has reached a distal most position at a distal end of the drive surface. In FIG. 39H, the representations 6862 and 6864 are shown in a fourth color, texture, and/or pattern to indicate, for example, that their corresponding interventional devices and/or hub assemblies may be experiencing a high force and/or resistance.

Additional details regarding control mechanisms and user interfaces are described in U.S. patent application Ser. No. 18/525,267, entitled METHOD FOR ROBOTICALLY CONTROLLING SUBSETS OF INTERVENTIONAL DEVICE ASSEMBLY, filed Nov. 30, 2023, the entirety of which is hereby incorporated by reference herein.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

The foregoing represents one specific implementation of a robotic control system. A wide variety of different robotic control system constructions can be made, for supporting and axially advancing and retracting two or three or four or more assemblies to robotically drive interventional devices, as will be appreciated by those of skill in the art in view of the disclosure herein.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 3°, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A robotic interventional device control system, comprising:
a first hub assembly coupled to a first interventional device, the first hub assembly comprising:
a first proximal end; and
a first distal end; and
a second hub assembly coupled to a second interventional device and positioned distal to the first hub assembly, the second hub assembly comprising:
a second proximal end; and
a second distal end;
a sensor system configured to detect a first position of the first hub assembly and a second position of the second hub assembly; and
one or more hardware processors configured to generate a user interface, the user interface comprising a drive table window, the drive table window comprising:
a first representation of the first hub assembly, the first representation of the first hub assembly comprising a first visual indication of the first distal end of the first hub assembly; and
a second representation of the second hub assembly, the second representation of the second hub assembly comprising a second visual indication of the second proximal end of the second hub assembly, wherein the second visual indication of the second proximal end of the second hub assembly is positioned relative to the first visual indication of the first distal end of the first hub assembly based on the detected first and second positions received from the sensor system, thereby the first and second visual indications provide an indication on the user interface how far apart the first distal end of the first hub assembly is from the second proximal end of the second hub assembly; and
a display configured to display the user interface.

2. The system of claim 1, wherein the first representation of the first hub assembly and the second representation of the second hub assembly are configured to transition from a first state to a second state upon an occurrence of a control system condition.

3. The system of claim 2, wherein the control system condition comprises a condition in which the first distal end of the first hub assembly is within a threshold distance of the second proximal end of the second hub assembly.

4. The system of claim 2, wherein the control system condition comprises a condition in which the first distal end of the first hub assembly abuts the second proximal end of the second hub assembly.

5. The system of claim 2, wherein the first state comprises a first color, texture, or pattern, and wherein the second state comprises a second color, texture, or pattern.

6. The system of claim 1, wherein the drive table window is configured to show an alert message upon an occurrence of a control system condition.

7. The system of claim 6, wherein the control system condition comprises a condition in which the first distal end of the first hub assembly is within a threshold distance of the second proximal end of the second hub assembly.

8. The system of claim 6, wherein the control system condition comprises a condition in which the first distal end of the first hub assembly abuts the second proximal end of the second hub assembly.

9. The system of claim 2, wherein the user interface comprises an instrument window, wherein the user interface is configured to transition from displaying the instrument window to displaying the drive table window upon an occurrence of the control system condition.

10. The system of claim 9, wherein the instrument window comprises:
a representation of the first interventional device, said representation of the first interventional device including a visual indication of a distal end of the first interventional device; and
a representation of the second interventional device, said representation of the second interventional device including a visual indication of a second distal end of the second interventional device.

11. The system of claim 1, further comprising a controller having one or more controls configured to cause movement of at least one of the first hub assembly and the second hub assembly.

12. The system of claim 11, further comprising a status indicator window comprising;
a representation of the first hub assembly;
a representation of the second hub assembly; and
a linkage indicator configured to provide a visual indication indicating that the first hub assembly and the second hub assembly are both linked to a first control of the controller.

13. The system of claim 12, wherein the status indicator window comprises a drive mode indicator configured to provide a visual indication indicating that the control system is controlling the first hub assembly and the second hub assembly in response to one or more inputs using the controller in accordance with a predefined drive mode.

14. The system of claim 13, wherein the predefined drive mode causes the first hub assembly and the second hub assembly to move simultaneously at different speeds in response to movement of a single control of the controller.

15. The system of claim 11, wherein the controller is configured to control axial movement of the first hub assembly and the second hub assembly along a drive table, wherein the first representation of the first hub assembly is configured to transition from a first configuration to a second configuration when the first hub assembly is moving axially along the drive table.

16. The system of claim 11, wherein the first representation of the first hub assembly is configured to transition from a first configuration to a second configuration when movement of the first hub assembly is linked to a first control of the controller.

17. The system of claim 1, further comprising a drive table, wherein the drive table window comprises a representation of the drive table including a visual indication of a first end of the drive table and a visual indication of a second end of the drive table.

18. The system of claim 17, wherein the sensor system is configured to detect a position of the drive table.

19. The system of claim 17, wherein the representation of the drive table is configured to move within the drive table window in response to movement of the drive table.

20. The system of claim 17, further comprising a telescoping member, wherein the drive table window comprises a representation of the telescoping member.
